(19) United States
(12) Patent Application Publication
(10) Pub. No.: US 2006/0178638 A1
(43) Pub. Date: Aug. 10, 2006

(54) Device and method for pharmaceutical mixing and delivery

(76) Inventor: David L. Reynolds, Bromont (CA)

Correspondence Address: BERESKIN AND PARR, 40 KING STREET WEST, BOX 401, TORONTO, ON, M5H 3Y2, CA

(21) Appl. No.: 11/292,124

(22) Filed: Dec. 2, 2005

Related U.S. Application Data
(60) Provisional application No. 60/632,530, filed on Dec. 3, 2004.
(60) Provisional application No. 60/645,531, filed on Jan. 21, 2005.

(51) Int. Cl. A61M 5/00

(52) U.S. Cl. 604/191

(57) ABSTRACT

Embodiments of the invention relate generally to cartridges, devices and methods for pharmaceutical constituents for storage, mixing and delivery. Particular embodiments relate to a device and method for pharmaceutical constituent mixing and delivery using a double-chambered cartridge within a socket member, for use as part of a syringe device. Other embodiments relate to a cartridge assembly for storing the pharmaceutical constituents prior to mixing and a method of forming the cartridge assembly. Still further embodiments relate to an extensible plunger for actuation of the delivery device. The extensible plunger is actuated in a first stroke while in its retracted state to mix the constituents and is actuated in a second stroke in its extended state to deliver the mixed constituents to an external volume.

### CROSS REFERENCE TO RELATED APPLICATIONS

This application claims the benefit of U.S. Provisional Patent Application Ser. No. 60/632,530, filed Dec. 3, 2004 and U.S. Provisional Patent Application 60/645,531, filed Jan. 21, 2005, the entire contents of both of which are hereby incorporated by reference.

### FIELD OF THE INVENTION

The present invention relates generally to cartridges, devices and methods for pharmaceutical constituent storage, mixing and delivery. In particular aspects, the invention relates to a device and method for pharmaceutical constituent mixing and delivery using a double-chambered syringe. In other aspects, the invention relates to a cartridge assembly for storing the pharmaceutical constituents prior to mixing and a method of forming the cartridge assembly.

### BACKGROUND OF THE INVENTION

In pharmaceutical delivery systems, it is sometimes necessary to mix a drug in powder form with a diluent in order to be able to deliver the drug to a subject. This can be done manually, such as by injecting the diluent into a vial containing the powdered drug, mixing the drug into the diluent and aspirating the drug in fluid form into a syringe for subsequent injection into the subject. Such manual procedures may be cumbersome and inconvenient and often lead to wastage of some of the drug as it may remain in the vial.

For dual chambered syringes using some form of outwardly extending bypass channel to provide fluid communication between the two chambers, the two chambers are commonly defined within the syringe barrel by two separate pistons and an end stopper spaced apart within the syringe barrel. In such an arrangement, the two chambers are placed in fluid communication via the bypass channel by actuation of the outermost piston within the syringe barrel, thereby pushing both pistons within the syringe barrel to a point where the innermost piston becomes aligned with the bypass channel. At that point, further actuation of the outermost piston causes fluid contained in the chamber between the two pistons to flow into the outwardly extending bypass channel and around the innermost piston (which remains stationary due to lack of an axial pushing force) via the bypass channel into the other chamber, where the fluid mixes with the constituent in that other chamber.

With the two chambered syringe barrel and external bypass arrangement described above, it is possible to push the innermost piston too far along the bypass channel, so that the piston again isolates the first and second chambers, but without having allowed full fluid transmission from the first chamber to the second chamber. This may be caused by an overly vigorous application of the plunger to the outermost piston. Once the innermost piston is pushed past the bypass channel without having allowed proper fluid flow from the first chamber to the second chamber, it may be difficult to recover the position of that piston to a bypass position allowing proper fluid communication between the chambers, as the innermost piston is not acted directly upon by the plunger. Even if the bypass position can be recovered, the mixing operation of the constituents may have been compromised by the improper actuation of the plunger.

With some pharmaceutical constituents, in order to properly constitute the pharmaceutical substance, it is desirable to ensure that the mixing of the two constituents occurs slowly and evenly. Thus, an overly vigorous application of the plunger to the outermost piston may result in mixing of the constituents in a suboptimal manner.

For pharmaceutical substances that rely on mixing a diluent with a drug in a dry powdered form, there can be problems experienced in filling the dry constituent into the container on an assembly line. If the neck of the container is too narrow, filling of the dry constituent is difficult and may take longer to fill the appropriate amount. Some vial and cartridge neck openings are small and thus are harder to fill with powder. For this reason, the active drug constituent is sometimes filled into the cartridge in liquid form and then converted to dry powder form by lyophilization. However, lyophilization involves freeze-drying the drug in fluid form to obtain the dry powdered form. In a double-chambered cartridge, the diluent, which is usually water, must be filled after lyophilization to avoid freezing thereof.

Lyophilization requires that the cartridge be aseptically transferred to a freeze dryer after the fluid drug constituent has been metered into the cartridge. In the freeze dryer, sublimation of the frozen drug solution takes place over many hours and sometimes days, which is a slow and expensive process when compared to instantly metered filling of the dry powdered drug.

With the lyophilization process, once the drug is lyophilized and sealed within the cartridge, the cartridge must be aseptically returned to a filling station for filling the diluent into a separate part of the cartridge. Since the diluent is filled after the lyophilized drug, and both are contained by the same glass barrel of the cartridge, sterilization of the diluent after filling it into the cartridge, for example, by an autoclave, may adversely affect the lyophilizate, as most lyophilizates are heat sensitive. Thus, where lyophilization is used as part of filling double-chambered cartridges, the diluent cannot be terminally sterilized after it is filled into the cartridge.

Further, while double-chambered cartridges can be used to mix two constituents of a pharmaceutical substance preparatory to injection, such devices are not suitable for mixing more than two constituents. If a third constituent is to be mixed with the pharmaceutical substance, this must be done manually according to conventional manual mixing techniques, as described above.

For double-chambered cartridges employing an external bypass, the external bypass protrudes somewhat from the cylindrical outer surface of the cartridge tube, resulting in an uneven cartridge profile. As some cartridge filling lines rely on gripping the cartridge tube around its cylindrical outer surface, the projecting external bypass may interfere with this gripping as it presents an uneven external surface, which does not appear to be plainly cylindrical to the gripping equipment. This can lead to handling errors or damage to the cartridge tube.

It is desired to address or ameliorate one or more of the shortcomings, disadvantages or problems described above, or to at least provide a useful alternative thereto.

### SUMMARY OF THE INVENTION

In one aspect, the invention relates to a device for delivery of a pharmaceutical substance, comprising:

a socket member having a distal end and an open proximal end and a hollow body extending therebetween, the distal end being closed but for a fluid delivery passage therein;

a tubular container having a fluid communication end and an actuation end and being receivable in the socket member so that the fluid communication end is disposed toward the distal end of the socket member, the container having a first closure member disposed at the fluid communication end, a second closure member disposed at the actuation end, a piston within the container intermediate the first and second closure members and bypass means or enabling fluid to bypass the piston when the piston is in a bypass position;

an activation cap disposed around the actuation end of the container, the activation cap comprising a sleeve extending along a proximal portion of an outer surface of the container and being at least partly receivable within the proximal end of the socket member, the activation cap further comprising a base portion arranged to grip the actuation end of the container and to permit access to the second closure member through the actuation end; and

a plunger engageable with the second closure member for moving the second closure member within the container;

wherein, in a pre-activated state of the device, a first chamber is defined by the container, the second closure member and the piston, a second chamber is defined by the container, the piston and the first closure member and the first chamber is sealed from communication with the fluid delivery passage of the socket member and, in an activated state of the device, the fluid delivery passage is in fluid communication with the second chamber through the first closure member for delivery of the contents of the second chamber through the fluid delivery passage.

Preferably, the plunger is longitudinally extensible between a retracted position and an extended position. Preferably, the plunger comprises first and second coaxial members. The first member includes a shaft portion and a head portion and the second member includes a sleeve portion and an engagement portion for engaging the second closure member. In the retracted position, the sleeve portion generally surrounds the shaft portion and in the extended position, the shaft portion is mostly withdrawn from the sleeve portion.

In one embodiment, the shaft portion and sleeve portion have mating male and female threads, respectively and the plunger is moved between the retracted position and the extended position by screw rotation of the shaft portion relative to the sleeve portion. In an alternative embodiment the shaft portion may be slidably moveable within the sleeve portion between the retracted and extended positions. In such an embodiment, the sleeve portion has inward radial portions at a proximal end of the sleeve portion for engaging proximal and distal engagement portions of the shaft in the retracted and extended positions, respectively. In further embodiments, the shaft and sleeve have cooperating structures to resist relative rotation between the shaft and sleeve.

Preferably, the socket member is generally tubular and resembles a syringe socket. In one embodiment, the socket member comprises puncturing means, preferably in the form of a hollow spike, comprising at least a part of the fluid delivery passage for puncturing the first closure member to thereby place the fluid delivery passage and fluid communication with the second chamber. Alternatively, the puncturing means may include a separately formed hollow needle received through the fluid delivery passage.

Generally, the container comprises a two-chambered cartridge, with the piston dividing the first and second chambers. In an activated position, the piston may be moved distally within the cartridge by actuating the plunger to drive the second closure member distally within the cartridge. Once the piston is aligned with the bypass channel (i.e. in the bypass position), fluid in the first chamber can be communicated to the second chamber around the piston via the bypass channel by further actuation of the plunger and corresponding insertion of the second closure member to drive it distally within the cartridge.

In a preferred embodiment, the first chamber holds a fluid constituent. The second chamber may hold a dry or fluid constituent. With the piston in the bypass position, the first constituent can be mixed with the second constituent by actuating the plunger and causing the first constituent to flow through the bypass channel into the second chamber. Once the first and second constituents have been mixed, the mixture can be communicated through the fluid delivery passage, for example to a patient or to a third chamber for further mixing.

In a further embodiment, the device further includes a connection part engagable with the closed end of the socket member and having a fluid connection passage for providing fluid communication between the fluid delivery passage and a third chamber. The third chamber holds a third constituent and may be retained by, or connectable to, the connection part. Thus, the device is, in one embodiment, a triple chamber device for mixing three separate constituents. The connection part of the device is removable so that, once the three constituents are mixed, the mixture may be aspirated back into the container for subsequent delivery to a patient, for example, through a needle connected to the closed end of the socket member.

In another aspect, the invention relates to a method of delivery of a substance, comprising the steps of:

providing a delivery device comprising a cartridge containing separate first and second constituents and at least partly received in the syringe socket, an axially extensible plunger extensible between a retracted position and an extended position and fluid connection means for providing fluid communication between a volume internal of the syringe socket and an external volume thereof;

activating the delivery device by inserting a part of the fluid connection means into the cartridge and thereby establishing fluid communication between an internal volume of the cartridge and the external volume;

actuating the plunger in its retracted position, in a first stroke of the plunger, to cause the first constituent to mix with the second constituent and form the substance;

extending the plunger to the extended position; and

actuating the plunger in its extended position, in a second stroke of the plunger, to deliver the substance to the external volume via the fluid connection means.

In another aspect, the invention relates to a method of mixing first and second constituents of a substance, comprising the steps of:

providing a delivery device comprising a socket assembly, a cartridge separately containing the first and second constituents and at least partly received in the socket assembly, a plunger engageable with a closure member of the cartridge and fluid connection means for providing fluid communication between a volume internal of the socket assembly and an external volume thereof the socket assembly having a base portion engaging a proximal end of the cartridge and defining a base opening;

activating the delivery device by inserting a part of the fluid connection means into the cartridge and thereby establishing fluid communication between an internal volume of the cartridge and the external volume;

actuating the plunger to move the closure member within the cartridge and to thereby cause the first constituent to mix with the second constituent and form the substance, such mixing being performed in a first stroke of the plunger; and

interfering the base portion of the socket assembly with an outer surface of the plunger during actuation of the plunger to retard movement of the plunger and the closure member during the first stroke.

In another aspect, the invention relates to a device for delivery of a pharmaceutical substance, comprising:

a syringe socket having a fluid connection end;

a cartridge at least partly received in the syringe socket, the cartridge defining a first chamber containing a first constituent, a second chamber containing a second constituent and a bypass channel for fluidly connecting the first and second chambers to allow mixing of the first and second constituents;

a connection part comprising fluid communication means and first and second engaging portions, the first engaging portion being adapted to engage the syringe socket at the fluid connection end and the second engaging portion being adapted to engage a head portion of a container having a third chamber containing a third constituent, wherein, in an activated position of the device, the second chamber is in fluid communication with the fluid communication means via the fluid connection end, whereby, when the head portion of the container is engaged with the second engaging portion and the fluid communication means is in fluid communication with the third chamber, mixed first and second constituent is fluidly mixable with the third constituent to form the pharmaceutical substance; and

means for aspirating the pharmaceutical substance into the cartridge for mixing and subsequent delivery of the pharmaceutical substance via the fluid connection end of the syringe socket.

In another aspect, the invention further relates to a device for mixing constituents of a pharmaceutical substance, comprising:

first, second and third chambers defined by the device, the first, second and third chambers containing respective first, second and third constituents and being oriented in sequence along a longitudinal axis of the device;

wherein the device is arranged such that, in an activated position, the first, second and third constituents may be mixed by actuation of a plunger upon a piston associated with the first chamber.

In the activated position, initial actuation of the plunger causes the first constituent to flow into the second chamber, thereby mixing the first and second constituents, and further activation of the plunger causes mixed first and second constituent to flow into the third chamber and thereby mix the first and second constituent with the third constituent. Still further actuation of the plunger causes mixed first, second and third constituent to flow into the second chamber.

The first, second and third chambers are preferably at least partly received within an elongate tubular housing. The housing preferably comprises first and second detachable parts, the first detachable part at least partly receiving the first and second chambers and the second detachable part at least partly receiving the third chamber.

Yet another aspect of the invention relates to an extensible plunger for a syringe, comprising:

an elongate first member;

an elongate second member having a piston engagement portion on a distal end thereof for engaging a piston of the syringe for actuation of the piston; and

wherein the first member is axially moveable relative to the second member between a retracted position, in which the first and second members substantially mate with each other, and an extended position, in which the second member substantially extends away from the first member.

Yet another aspect of the invention relates to a cartridge tube for a pharmaceutical cartridge, the cartridge tube comprising:

a hollow tube, the hollow tube having a wall and opposed first and second ends; and

a bypass portion formed in the wall, intermediate the first and second ends, the bypass portion comprising at least one inward deformation of the wall.

Preferably, the at least one inward deformation is rounded and extends substantially radially inwardly relative to a nominal internal surface of the wall. Preferably, a contour (including the outer surface contour) of the wall is at least partially inwardly deformed at the bypass portion. The at least one inward deformation preferably extends inwardly of the nominal internal surface of the wall by between 0.7 mm and 1.2 mm. More preferably, this inward extension is between 0.8 mm and 1.0 mm. Even more preferably, this inward extension is about 0.9 mm. The width of the at least one inward deformation is preferably about 2.0 mm to 2.5 mm, more preferably about 2.25 mm. The length of the at least one inward deformation is preferably about 12.5 mm to 14.0 mm, more preferably about 13.3 mm.

Preferably, the hollow tube is formed of glass. The glass may be borosilicate glass and may contain cerium oxide. Alternatively, the hollow tube may be formed of a suitable plastic.

In one embodiment, the bypass portion comprises two inward deformations of the wall. In a further embodiment, more than two inward deformations of the wall may be provided. Preferably, the two inward deformations are adjacent each other. Alternatively, the two inward deformations may be spaced from each other. The two inward deformations are preferably substantially parallel and longitudinally oriented. The two inward deformations are arranged so that they define at least one bypass channel therebetween. When a deformable piston is received within the bypass portion, the two inward deformations cause the piston to radially inwardly deform and partially separate from the wall, thereby allowing fluid flow along the at least one bypass channel. Up to three bypass channels may be defined by the two inward deformations and the wall when the piston is received within the bypass portion.

The hollow tube is preferably adapted to receive a closure member, such as a stopper, for example, at least partially within a neck portion of the second end. The neck portion may be straight or, alternatively, may be circumferentially detented and circumferentially flanged.

Yet another aspect of the invention relates to a filled cartridge for mixing pharmaceutical constituents therewithin, the cartridge comprising:

a hollow tube, the hollow tube having a wall and opposed first and second ends;

a bypass portion formed in the wall intermediate the first and second ends, the bypass portion comprising at least one inward deformation of the wall;

a first piston disposed within the hollow tube between the bypass portion and the first end;

a second piston disposed within the hollow tube away from the bypass portion and toward the first end so that the first and second pistons define a first chamber therebetween;

a first constituent disposed in the first chamber;

a closure disposed at least partially within the second end so that the first piston and the closure define a second chamber therebetween; and

a second constituent disposed in the second chamber;

wherein the first and second pistons are movable within the hollow tube and wherein, when the first piston is received within the bypass portion, at least one bypass channel is formed between the first piston and the wall, thereby allowing fluid communication between the first and second chambers for mixing the first and second constituents.

A further aspect of the invention relates to a cartridge assembly comprising the filled cartridge described above and a support cap. The support cap is disposed around, and engages, the first end of the hollow tube. The support cap has a base portion comprising an apron of greater diameter than an outside diameter of the hollowed tube and having a footprint of substantially circular shape, for example, of an annular shape, when the cartridge assembly is positioned upright so that the filled cartridge is disposed vertically.

Yet another aspect of the invention relates to a partially filled cartridge, the cartridge comprising:

a hollow tube, the hollow tube having a wall and opposed first and second ends;

a bypass portion formed in the wall intermediate the first and second ends, the bypass portion comprising at least one inward deformation of the wall;

a first piston disposed within the hollow tube between the bypass portion and the first end;

a second piston disposed within the hollow tube away from the bypass portion end toward the first end so that the first and second pistons define

a first chamber therebetween; and

a first constituent disposed in the first chamber;

wherein the first and second pistons are movable within the hollow tube and wherein, when the first piston is received within the bypass portion, at least one bypass channel is formed between the first piston and the wall, thereby allowing fluid communication between the first chamber and a second chamber, the second chamber being defined by the first piston, the wall and the second end.

A still further aspect of the invention relates to a cartridge assembly comprising the partially filled cartridge described above and a support cap as described above.

A still further aspect of the invention relates to a method of forming a cartridge tube, comprising the steps of:

providing a hollow tube having a wall and first and second ends; and

deforming the wall inwardly from the outside of the hollow tube so as to form an inward deformation of an internal surface of the wall.

Preferably, the method further comprises, prior to the step of deforming, the step of thermally softening the hollow tube and, after the step of deforming, the step of thermally setting the inward deformation. The inward deformation is preferably formed longitudinally, intermediate the first and second ends, using a pressing tool. The step of deforming may include supporting the internal surface of the wall adjacent the inward deformation while pressing inwardly from the outside.

A still further aspect of the invention relates to a method of forming a cartridge assembly, comprising the steps of:

providing a cartridge tube, the cartridge tube being hollow and having a wall, first and second ends and a bypass portion, the bypass portion being formed intermediate the first and second ends;

inserting a first piston in the cartridge tube intermediate the first end and the bypass portion;

orienting the cartridge tube so that the first end is upward of the second end;

filling a first constituent into the cartridge tube between the first piston and the first end;

inserting a second piston into the cartridge tube between the first constituent and the first end so that the first constituent is fluidly sealed between the first and second pistons; and

fitting the first end of the cartridge tube into a support cap to form a partially filled cartridge assembly, the support cap having a support footprint substantially larger than a tube footprint of the cartridge tube, whereby the cartridge tube can be transported in an upright manner while supported by the support cap.

Preferably, the method of forming a cartridge assembly further comprises the steps of:

transporting the partially filled cartridge assembly to a powder filling facility;

sterilizing the partially filled cartridge assembly; and

filling a second constituent into the cartridge tube between the first piston and the second end, the second constituent being in powder form.

The method of forming a cartridge may further comprise the step of sealing the second end to form a filled cartridge assembly. The filled cartridge assembly may then be inserted into a hollow syringe socket so that the second end is received within a distal socket and the support cap is partially received within a proximal end of the syringe socket.

A still further aspect of the invention relates to a method of assembling a filled cartridge, the method comprising:

forming at a first filling facility a partially filled cartridge, the partially filled cartridge defining a sealed first chamber containing a first constituent and an open second chamber;

transporting the partially filled cartridge to a second filling facility;

sterilizing the partially filled cartridge;

filling a second constituent into the second chamber; and

sealing the second chamber.

A still further aspect of the invention relates to a method of forming a cartridge tube, comprising the steps of:

providing a hollow tube having a wall and first and second ends; and

deforming the wall inwardly from the outside of the hollow tube so as to form first and second inward deformations of an internal surface of the wall.

The step of deforming defines a bypass portion along the wall. The first and second inward deformations are preferably formed longitudinally, intermediate the first and second ends, using a pressing tool. The first and second inward deformations may be formed simultaneously, or, alternatively, may be formed in sequence. The first and second inward deformations are preferably formed adjacently so as to have an angular separation, relative to a center of a hollow tube, between about 30 to 40 degrees. The angular separation is more preferably about 35 degrees. The first and second inward deformations are formed so as to define a channel therebetween.

Certain embodiments of the invention enable first and second constituents of a pharmaceutical to be easily mixed and delivered to a patient. The structure of the extensible plunger arrangement allows for the constituents to be mixed during a first stroke of the plunger (in its retracted position), and for the mixed constituents to be delivered through the fluid delivery passage during a second stroke of the plunger (in its extended position).

Certain embodiments of the invention provide interfering contact of an outer surface of the plunger with a base portion of the activation cap as the plunger is actuated in a first stroke (i.e. during mixing of the first and second constituents). This interfering contact serves to retard movement of the plunger during actuation thereof and thus mitigate against an overly vigorous actuation of the plunger when mixing the first and second constituents. In certain embodiments, this interfering contact is provided by a tapered outer surface of the plunger, such that the outer surface of the plunger provides progressively interfering contact, and thus increasing resistance, as the plunger is further actuated. In other embodiments, the outer surface of the plunger is provided with circumferential or partly circumferential ridges or corrugations for interfering with the base grips of the activation cap during actuation of the plunger and thus slowing or retarding the actuating movement thereof. Further, in some embodiments, the plunger may be extensible, while in other embodiments, the plunger is inextensible.

Other embodiments of the invention enable three constituents to be easily mixed and delivered to a patient using a double-chambered cartridge within the syringe socket to mix the constituents thereof with the contents of a third chamber held by a connector removably engaged with the syringe socket.

Still other embodiments of the invention provide a double-chambered cartridge having a relatively large neck opening for increased ease of filling the dry constituent. In one embodiment, the wider neck is achieved by having the neck of the cartridge be the same inner diameter as the rest of the cartridge, thus avoiding the narrow neck structure associated with conventional cartridges and vials.

Further embodiments of the invention relate to a cartridge tube and corresponding cartridge, cartridge assembly and forming methods employing an internal bypass portion formed in the cartridge tube. The internal bypass avoids having an external bypass extending outwardly from the external cylindrical surface of the cartridge tube and thus avoids the handling problems during cartridge assembly associated therewith. The internal bypass may be formed in a simple and cost-effective manner by deforming the cartridge tube wall inwardly from the outside of the cartridge tube to form an inward deformation of the internal surface of the tube. More than one such internal deformation may be formed so as to define one or more bypass channels without the necessity of an external projection from the cartridge tube wall.

Further embodiments relate to methods of cartridge assembly which involve forming a partially filled cartridge assembly, transporting it, sterilizing it and completing the filling of the cartridge assembly at a destination location. This is advantageous where powder filling is involved, as it can be problematic to perform liquid and powder filling operations within the same filling facility without the powder contaminating the liquid filling process. The partially filled cartridge (being filled with diluent) according to one embodiment of the invention can advantageously be sterilized at the powder filling facility prior to the powder filling operation.

Further features and advantages of embodiments of the invention are described in the following detailed description or may be evident therefrom.

### BRIEF DESCRIPTION OF THE DRAWINGS

Embodiments of the invention are described in further detail below, by way of example only, with reference to the drawings, in which:

FIG. 1A is a side view of one embodiment of a delivery device and a needle;

FIG. 1B is a side sectional view of the delivery device and needle of FIG. 1A;

FIG. 1C is an exploded perspective view of the delivery device and needle of FIG. 1A;

FIG. 1D is an exploded side sectional view of the delivery device and needle of FIG. 1A;

FIG. 1E is a side sectional view of the delivery device and needle of FIG. 1A in an activated state;

FIG. 2A is a side sectional view of the delivery device and needle of FIG. 1A and a plunger;

FIG. 2B is a side sectional view of the delivery device and needle of FIG. 2A in an activated state;

FIG. 2C is a side sectional view showing the plunger engaged with the delivery device of FIG. 2B in an activated state;

FIG. 2D is a side sectional view showing the delivery device, needle and plunger of FIG. 2C, with the plunger actuated in a first stroke;

FIG. 2E is a side sectional view showing the delivery device, needle and plunger of FIG. 2D, with the plunger in an extended position;

FIG. 2F shows the delivery device, needle and plunger of FIG. 2E, with the plunger actuated in a second stroke and with the needle cap removed for fluid delivery;

FIG. 3A is a side view of one embodiment of a plunger in a retracted position;

FIG. 3B is a side sectional view of the plunger, taken along the line 1-1 of FIG. 3A;

FIG. 3C is a perspective view of the plunger shown in FIG. 3A;

FIG. 4A is a side view of the plunger of FIG. 3A, shown in an extended position;

FIG. 4B is a side sectional view of the plunger, taken along line 2-2 of FIG. 4A;

FIG. 4C is a perspective view of the plunger shown in FIG. 4A;

FIG. 5A is a side sectional view of the delivery device and needle of FIG. 1A and an alternative plunger;

FIG. 5B is a side sectional view of the delivery device and needle of FIG. 5A in an activated state;

FIG. 5C is a side sectional view showing the alternative plunger engaged with the delivery device of FIG. 5B in an activated state;

FIG. 5D is a side sectional view showing the delivery device, needle and plunger of FIG. 5C, with the plunger actuated in a first stroke;

FIG. 5E is a side sectional view showing the delivery device, needle and plunger of FIG. 5D, with the plunger in an extended position;

FIG. 5F shows the delivery device, needle and plunger of FIG. 5E, with the plunger actuated in a second stroke and with the needle cap removed for fluid delivery;

FIG. 6A is a side view of the alternative plunger in a retracted position;

FIG. 6B is a side sectional view of the alternative plunger taken along the line 3-3 of FIG. 6A;

FIG. 6C is a perspective view of the alternative plunger of FIG. 6A;

FIG. 7A is a side view of the alternative plunger of FIG. 6A, shown in an extended position;

FIG. 7B is a side sectional view of the alternative plunger, taken along line 4-4 of FIG. 7A;

FIG. 7C is a perspective view of the alternative plunger of FIG. 7A;

FIG. 8A is a side view of one embodiment of a syringe socket;

FIG. 8B is a side sectional view of the syringe socket of FIG. 8A, taken along the line 5-5;

FIG. 8C is a perspective view of the syringe socket shown in FIG. 8A;

FIG. 9A is a side view of an embodiment of an activation cap;

FIG. 9B is a sectional view of the activation cap, taken along the line 6-6 of FIG. 9A;

FIG. 9C is a rear sectional view of the activation cap, taken along the line 7-7 of FIG. 9B;

FIG. 9D is a bottom perspective view of the activation cap shown in FIG. 9A;

FIG. 9E is a top perspective view of the activation cap of FIG. 9A;

FIG. 10A is a side view of a further embodiment of a delivery device and needle;

FIG. 10B is a side sectional view of the delivery device and needle shown in FIG. 10A;

FIG. 10C is an exploded perspective view of the delivery device and needle shown in FIG. 10A;

FIG. 10D is an exploded side sectional view of the delivery device and needle of FIG. 10A;

FIG. 10E is a side sectional view of the delivery device and needle of FIG. 10A in an activated state;

FIG. 11A is a side view of a further embodiment of a delivery device and needle;

FIG. 11B is a side sectional view of the delivery device and needle shown in FIG. 11A;

FIG. 11C is an exploded perspective view of the delivery device and needle shown in FIG. 11A;

FIG. 11D is an exploded side sectional view of the delivery device and needle of FIG. 11A;

FIG. 11E is a side sectional view of the delivery device and needle of FIG. 11A in an activated state;

FIG. 12A is a side view of a further embodiment of a delivery device and needle;

FIG. 12B is a side sectional view of the delivery device and needle shown in FIG. 12A;

FIG. 12C is an exploded perspective view of the delivery device and needle shown in FIG. 12A;

FIG. 12D is an exploded side sectional view of the delivery device and needle of FIG. 12A;

FIG. 12E is a side sectional view of the delivery device and needle of FIG. 12A in an activated state;

FIG. 13A is a side view of a further embodiment of a delivery device and needle;

FIG. 13B is a side sectional view of the delivery device and needle shown in FIG. 13A;

FIG. 13C is an exploded perspective view of the delivery device and needle shown in FIG. 13A;

FIG. 13D is an exploded side sectional view of the delivery device and needle of FIG. 13A;

FIG. 13E is a side sectional view of the delivery device and needle of FIG. 13A in an activated state;

FIG. 14A is a front view of another embodiment of an activation cap;

FIG. 14B is a sectional view of the activation cap, taken along the line 8-8 of FIG. 14A;

FIG. 14C is a sectional view of the activation cap, taken along the line 9-9 of FIG. 14B;

FIG. 14D is a bottom perspective view of the activation cap shown in FIG. 14A;

FIG. 14E is a top perspective view of the activation cap of FIG. 14A;

FIG. 15A is a side sectional view of a further embodiment of a delivery device;

FIG. 15B is an exploded perspective view of the delivery device shown in FIG. 15A;

FIG. 15C is an exploded side sectional view of the delivery device shown in FIG. 15A;

FIG. 15D is a side sectional view of the delivery device of FIG. 15A, shown in an activated state;

FIG. 16A is a side sectional view of one embodiment of a mixing device incorporating the delivery device of FIG. 15A;

FIG. 16B is an exploded perspective view of the mixing device shown in FIG. 16A;

FIG. 16C is an exploded side sectional view of the mixing device shown in FIG. 16A;

FIGS. 17A to 17F show sequential steps of one method of using the mixing device shown in FIG. 16A with the plunger shown in FIGS. 3A to 3C and 4A to 4C;

FIGS. 18A to 18D are partial side sectional views of one embodiment of a container used with the delivery device, showing sequential steps of one method of mixing and delivering first and second constituents in the container;

FIG. 19A is a side sectional view of a further embodiment of a delivery device having modified pistons;

FIG. 19B is a side sectional view of the delivery device shown in FIG. 19A, but with the modified pistons shown engaged;

FIG. 20A is a side view of one embodiment of a piston having a projection on one face thereof;

FIG. 20B is a side section view of the piston of FIG. 20A, taken along line 10-10;

FIG. 20C is a perspective view of the piston of FIG. 20A;

FIG. 21A is a side view of another embodiment of a piston having a recess in one face thereof;

FIG. 21B is a side sectional view of the piston of FIG. 21A, taken along line 11-11;

FIG. 21C is a perspective view of the piston of FIG. 21A;

FIGS. 22A to 22F are partial side sectional views of the mixing device of FIG. 16A, with the plunger of FIGS. 3A to 3C and 4A to 4C, showing sequential steps of another method of using the mixing device;

FIG. 23A is a plan view of a fluid connector used in the mixing device of 16A;

FIG. 23B is a side sectional view of the fluid connector, taken along line 12-12 of FIG. 23A;

FIG. 23C is a side sectional view of the fluid connector, taken along line 13-13 of FIG. 23A; and

FIG. 23D is a perspective view of the fluid connector of FIG. 23A;

FIG. 24A is a side sectional view of an alternative cartridge barrel;

FIG. 24B is an end section view of the alternative cartridge barrel of FIG. 24A, taken along line 14-14;

FIG. 24C is an end section view corresponding to FIG. 24B, but showing a piston within the cartridge barrel;

FIG. 25A is a side cross-sectional view of a further alternative plunger;

FIG. 25B is a perspective view of the plunger of FIG. 25A;

FIG. 26A is a side sectional view of a further alternative cartridge barrel;

FIG. 26B is an end sectional view of the further alternative cartridge barrel of FIG. 26A, taken along the line 15-15;

FIG. 26C is an end sectional view corresponding to FIG. 26B, but showing a piston within the cartridge barrel;

FIG. 27 is a side sectional view of a further alternative cartridge barrel;

FIG. 28 is a side sectional view of a further alternative cartridge barrel;

FIG. 29 is a process flow diagram of a method of assembling a cartridge assembly;

FIG. 30 is a pictorial process flow diagram of a method of assembling a cartridge assembly;

FIG. 31 is a side sectional view of the cartridge assembly in an upright position;

FIG. 32 is a side sectional view of another embodiment of a delivery device;

FIG. 33 is a side cross-sectional view of another embodiment of a delivery device;

FIG. 34A is a side cross-sectional view of a further alternative cartridge barrel;

FIG. 34B is an end sectional view of the further alternative cartridge barrel of FIG. 34A, taken along the line 16-16;

FIG. 35A is a side view of a syringe socket according to another embodiment;

FIG. 35B is a cross-sectional view of the syringe socket of FIG. 35A, taken along the line 17-17;

FIG. 35C is a perspective view of the syringe socket of FIG. 35A;

FIG. 36A is a side view of an activation cap according to another embodiment;

FIG. 36B is a cross-sectional view of the activation cap of FIG. 36A, taken along the line 18-18;

FIG. 36C is a perspective view of the activation cap of FIG. 36A;

FIG. 36D is a further perspective view of the activation cap of FIG. 36A;

FIG. 37A is a side view of an extensible plunger according to another embodiment, shown in a retracted position;

FIG. 37B is a cross-sectional view of the extensible plunger of FIG. 37A, taken along the line 19-19;

FIG. 37C is a perspective view of the extensible plunger of FIG. 37A;

FIG. 38A is a side view of the extensible plunger of FIGS. 37A to 37C, shown in an extended position;

FIG. 38B is a cross-sectional view of the extensible plunger of FIG. 38A, taken along the line 20-20;

FIG. 38C is a perspective view of the extensible plunger of FIG. 38A, shown in its extended position;

FIG. 39A is a side view of an extensible plunger according to another embodiment, shown in a retracted position;

FIG. 39B is a cross-sectional view of the extensible plunger of FIG. 39A, taken along the line 21-21;

FIG. 39C is a perspective view of the extensible plunger of FIG. 39A;

FIG. 39D is an end cross-sectional view of a sleeve of the extensible plunger of FIG. 39A;

FIG. 40A is a side view of the extensible plunger of FIGS. 39A to 39C, shown in an extended position;

FIG. 40B is a cross-sectional view of the extensible plunger of FIG. 40A, taken along the line 22-22;

FIG. 40C is a perspective view of the extensible plunger of FIG. 40A, shown in its extended position;

FIG. 41A is a side view of an extensible plunger according to another embodiment, shown in a retracted position;

FIG. 41B is a cross-sectional view of the extensible plunger of FIG. 41A, taken along the line 23-23;

FIG. 41C is a perspective view of the extensible plunger of FIG. 41A;

FIG. 41D is an end cross-sectional view of a sleeve of the extensible plunger of FIG. 41A;

FIG. 42A is a side view of the extensible plunger of FIGS. 41A to 41C, shown in an extended position;

FIG. 42B is a cross-sectional view of the extensible plunger of FIG. 42A, taken along the line 24-24; and

FIG. 42C is a perspective view of the extensible plunger of FIG. 42A, shown in its extended position.

### DETAILED DESCRIPTION OF EMBODIMENTS OF THE INVENTION

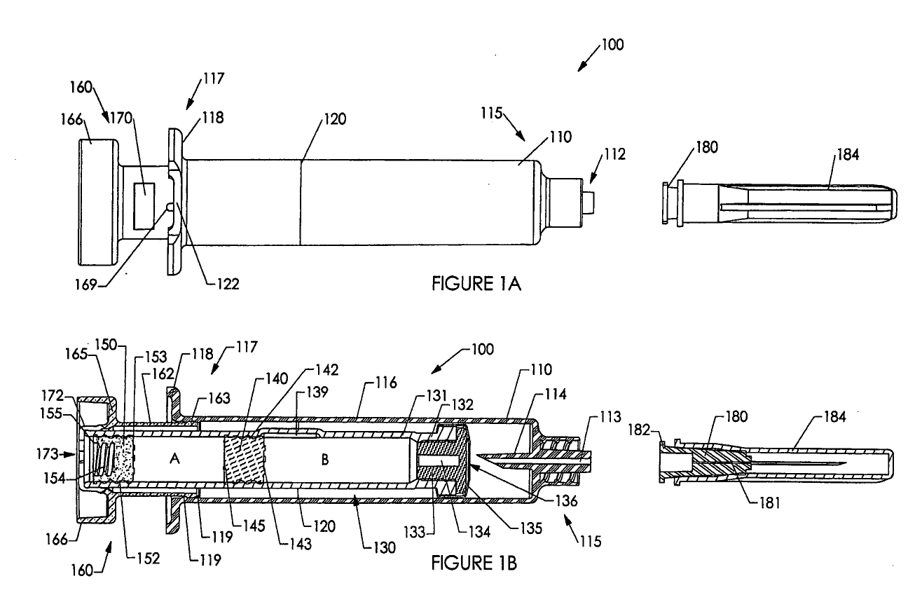

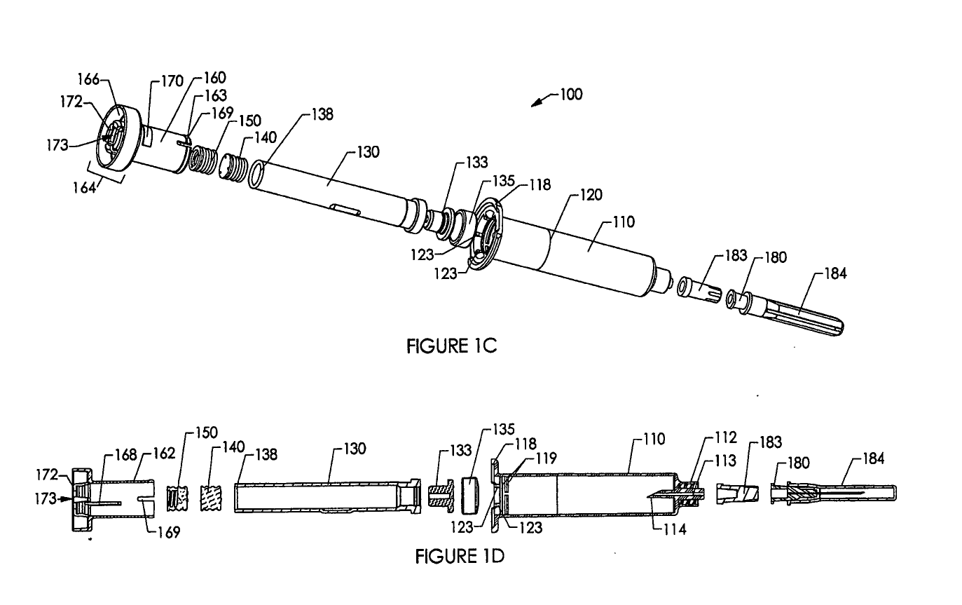

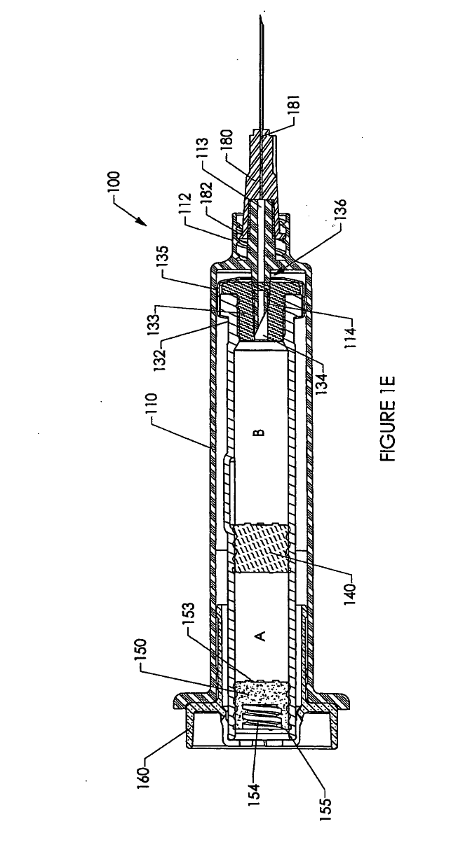

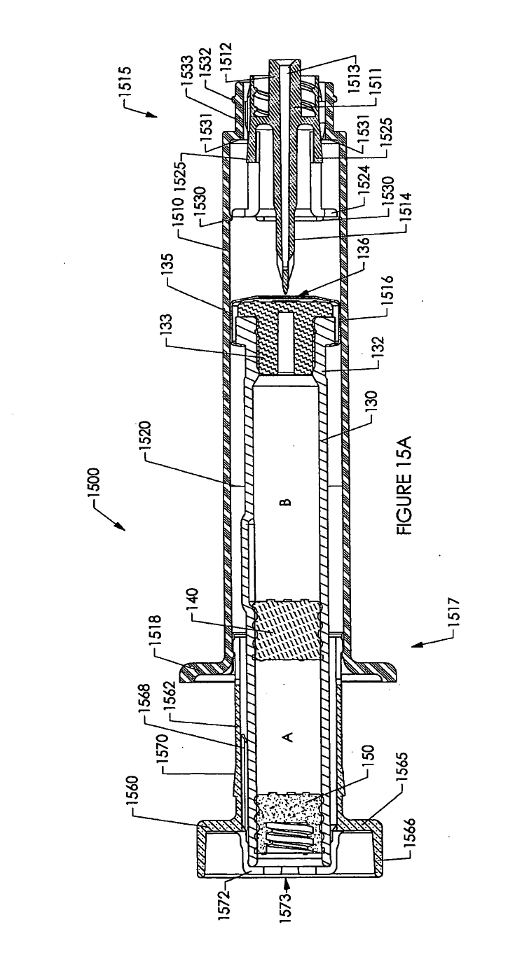

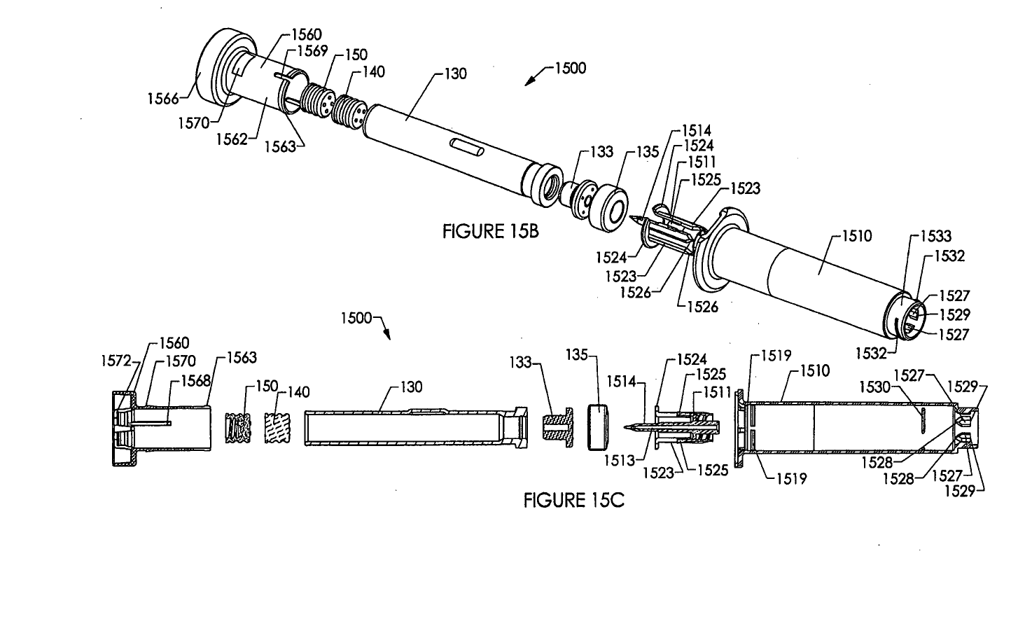

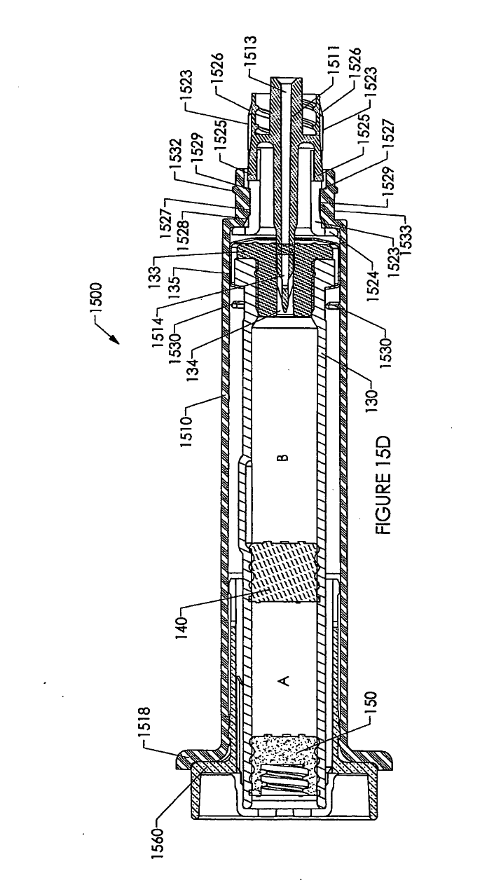

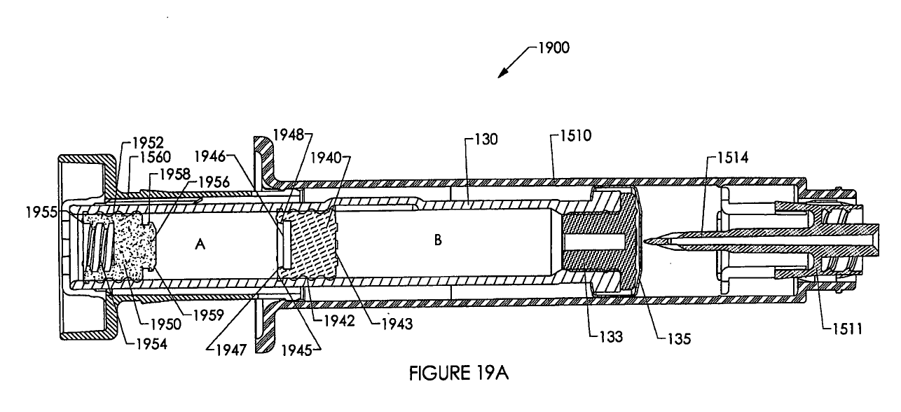

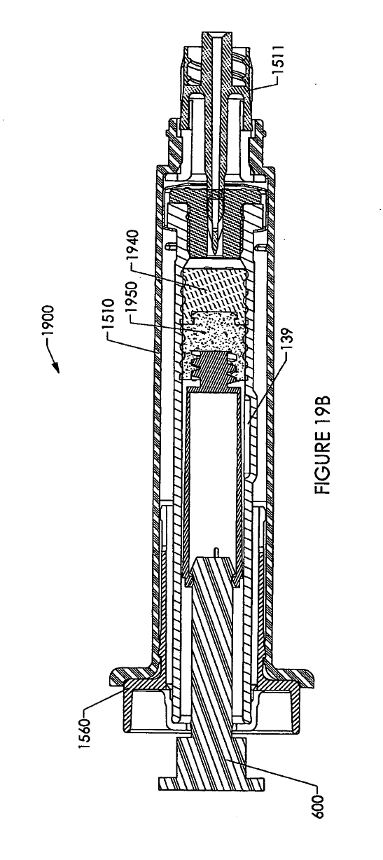

Various embodiments of the invention are shown in the drawings. Generally, like reference numerals are used, as between the drawings, to indicate like or similar features or functions. Further, where a particular feature or part is introduced in a drawing, the reference numeral for that feature or part begins with the figure number in the hundreds column. For example, for a feature introduced in FIG. 19, the reference numeral will be in the 1900s.

Where possible, reference numerals having the same number between 0 and 99, but having different increments of 100, are used to indicate like features or functions as between different embodiments shown in the drawings. For example, a syringe socket 110 is shown in FIGS. 1A to 1E, while an alternative syringe socket 1510 of another embodiment is shown in FIGS. 15A to 15D.

Throughout this specification, the term “distal” will be used to indicate a position, location or direction generally away from a person's hand while gripping the device toward its (proximal) base. The term “proximal” is used conversely to indicate a position, location or direction opposite to “distal” and more toward the hand of the person using the device and gripping it at its base. Generally, the distal end of a device described herein will correspond to a fluid delivery or connection end of the device and the proximal end of the device will correspond to the actuation end. Thus, “proximal” and “distal” are used as relative terms to indicate relative position, location or direction for ease of description and are not intended to be limiting on the features, functions or scope of protection sought. In the drawings, unless there is a contrary indication, proximal generally corresponds to a left direction and distal generally corresponds to a right direction.

Embodiments of the invention generally relate to methods and devices for mixing constituents, for example of a drug, in preparation for injection or other form of delivery of the mixed constituents into a patient or other subject. At least one of the constituents is a fluid diluent, for example, such as water.

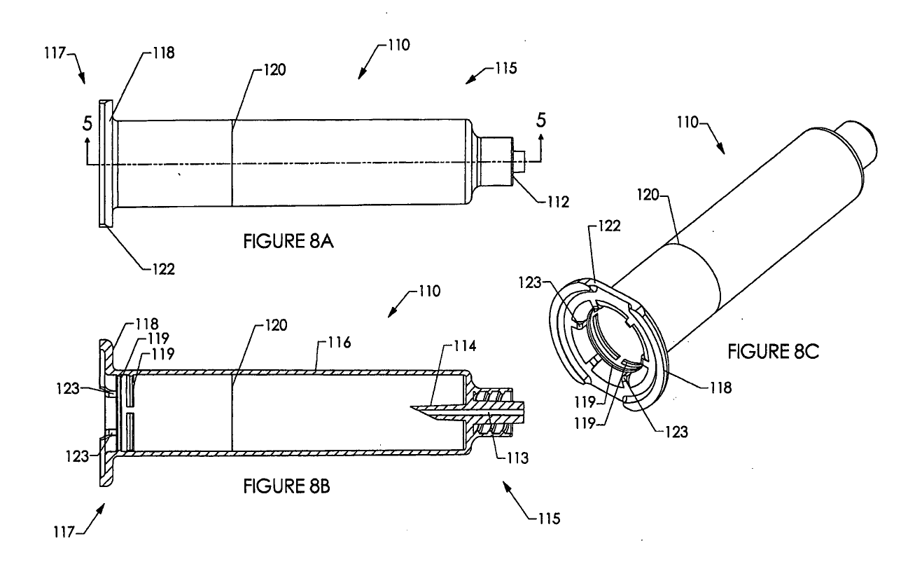

Referring to FIGS. 1A, 1B, 1C, 1D and 1E, there is shown a delivery device 100 according to one embodiment. The delivery device 100 comprises a syringe socket 110 having a distal end 115 and a proximal end 117. Delivery device 100 further includes a cartridge or container 130 mostly enclosed within the syringe socket 110 and an activation cap 160 surrounding one end of the cartridge 130 and being partly received within the syringe socket 110. A needle 180 is engageable with the syringe socket 110 at its distal end 115 for enabling delivery of a pharmaceutical substance contained in delivery device 100 to a subject. Syringe socket 110 is shown in further detail in FIGS. 8A to 8C.

The syringe socket 110 has a male luer fitting 112 at its distal end 115. The male luer fitting 112 is engageable with a female luer structure 182 on needle 180. Distal end 115 of syringe socket 110 has a fluid passage 113 extending therethrough so as to provide fluid communication from within the body of the syringe socket 110 through an opening in male luer fitting 112.

Distal end 115 further includes a spike 114 extending proximally inside a wall 116 of the syringe socket 110. Spike 114 is used to penetrate a penetrable seal in a stopper 133 of cartridge 130 for providing fluid communication between the inside of cartridge 130 and the opening in male luer fitting 112 via fluid passage 113. Spike 114 has a sharpened tip for penetrating stopper 133 and has an opening to fluid passage 113 toward the sharpened tip. Spike 114 is of a generally hollow tubular form and the sharpened tip (as shown in FIG. 1B) resembles that formed by an angled slice through such a hollow tubular member. Alternatively, the tip of spike 114 may be formed differently (such as is shown by spike 1514 in FIG. 15A), as long as it performs the function of penetrating the stopper or other closure member of the cartridge and establishing fluid communication from within the cartridge through the fluid delivery end.

Proximal end 117 of syringe socket 110 has an end flange 118 extending generally radially from the end of syringe socket 110. End flange 118 may be used by a person using delivery device 100 to place the fingers of a hand thereon and thus grip or press against end flange 118 during activation or actuation of the device or for otherwise causing relative movement of parts of the device or for assisting in gripping or holding the device. End flange 118 has flattened portions 122 formed on diametrically opposite sides of end flange 118. The flattened portions 122 help to limit any rolling of the delivery device 100 that may occur when it is placed on its side on a flat surface.

Syringe socket 110 is generally of a hollow tubular form, with wall 116 extending between distal end 115 and proximal end 117. Syringe socket 110 has a tapered form extending from taper transition point 120 to distal end 115 to assist in directing the head of the cartridge 130 centrally as it is pushed toward spike 114. This taper is quite shallow, being in the order of, for example, between 0.1 degrees and 0.5 degrees, but preferably about 0.3 degrees. Between taper transition point 120 and proximal end 117, wall 116 is not tapered because a sleeve of the activation cap must be receivable in this part of the syringe socket 110.

Internally of wall 116 toward proximal end 117, the syringe socket 110 has internally protruding ribs 119 extending circumferentially. Internal ribs 119 are preferably longitudinally spaced, but substantially adjacent one another so as to define a circumferential groove therebetween on the inside of wall 116. Preferably, only two internal ribs 119 are provided, although more than two such ribs may be provided, for example if it is desired to define more than one circumferential groove between the ribs. Internal ribs 119 may extend around the entire internal circumference of wall 116 or may be circumferentially discontinuous, so long as they sufficiently define the internal circumferential groove.

Cartridge 130 has a generally hollow tubular wall 131 formed of glass and having openings at each end. A neck portion 132 is provided at a distal end of cartridge 130. At the neck portion 132, wall 131 jogs slightly inwardly on its external surface, which then widens outwardly to create a flange or lip. Neck portion 132 may be of a standard form (13 millimeters outside diameter) for cartridges and vials.

The distal end of cartridge 130 is closed by stopper 133, which has an aluminum cartridge cap 135 fitted over the stopper and around the lip or flange of neck portion 132. Cartridge cap 135 has an opening 136 over the portion of stopper 133 which is to receive, and be penetrated by, spike 114 when delivery device 100 is placed in its activated position (as shown in FIG. 1E). Thus, cartridge cap 135 serves to retain stopper 133 within the neck of cartridge 130, while exposing a central portion of stopper 133 for penetration thereof.

Stopper 133 is formed of a compressible and sterilizable rubber, preferably a bromobutyl rubber. Alternatively, stopper 133 may be formed of chlorobutyl or halobutyl rubber or any other pharmaceutically acceptable rubber formulation. Stopper 133 has a stopper cavity 134 formed in a longitudinal orientation in the centre of the stopper, extending from a proximal end of the stopper towards a distal end thereof, while leaving a thin portion of rubber at the distal end for spike 114 to easily penetrate through. Stopper cavity 134 is sized so as to accommodate, in an interference fit, the outer diameter of spike 114 when delivery device 100 is in the activated position. Thus, the interior volume of cartridge 130 communicates with stopper cavity 134 and, when spike 114 is inserted in stopper cavity 134, the internal volume of cartridge 130 communicates to the outside of syringe socket 110 through fluid passage 113.

Spike 114 is sized so that, when it is received within stopper cavity 134 in the activated position, spike 114 substantially occludes stopper cavity 134 so as to limit the amount of space around spike 114 in which the mixed constituent may accumulate. Thus the substantial occlusion of stopper cavity 134 by spike 114 serves to minimize wastage of the mixed constituents, which is usually a valuable drug, by minimizing the volume in which the mixed constituents can remain within cartridge 130 without being delivered to the subject.

Cartridge 130 further includes a central piston 140 disposed within the tubular wall 131 in a compressive interference fit. Movement of central piston 140 thus requires a force sufficient to overcome the friction between the piston outer surface and the tubular wall 131. Central piston 140 is formed of a similar material to stopper 133. Central piston 140 has a ribbed outer wall 142 which is slightly compressed by wall 131 and which forms a liquid impermeable seal therewith. Piston 140 has a distal surface 143 and a proximal surface 145 disposed opposite each other and generally in a plane transverse to the longitudinal orientation of delivery device 100.

A second piston 150, also called an actuating piston or a closure member, is located toward the proximal end of cartridge 130 within wall 131 near the proximal end opening thereof. Actuating piston 150 is formed of a similar material to that of stopper 133 and central piston 140. Actuating piston 150 is similar to central piston 140 in structure, except that it has a plunger socket 154 therein, accessible through proximal surface 155. Plunger socket 154 has female screw threads for receiving male screw threads on the end of a plunger so that the plunger can removably engage actuating piston 150. Actuating piston 150 has an outer wall 152 compressible in an interference fit with the inner surface of wall 131 of cartridge 130 to form a liquid impermeable seal therewith.

A distal surface 153 of actuating piston 150 cooperates with the proximal surface 145 of central piston 140 and the interior surface of wall 131 to define a first chamber for containing a first constituent A. Distal surface 143 of central piston 140 cooperates with stopper 133 and the inner surface of wall 131 to define a second chamber for containing a second constituent B. Both the first and second chambers are collapsible upon actuation of pistons 150 and 140.

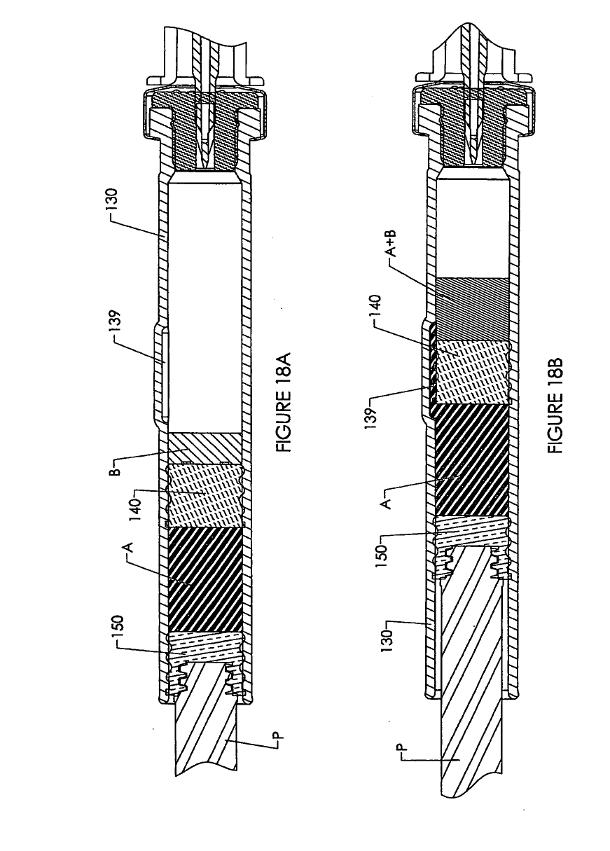

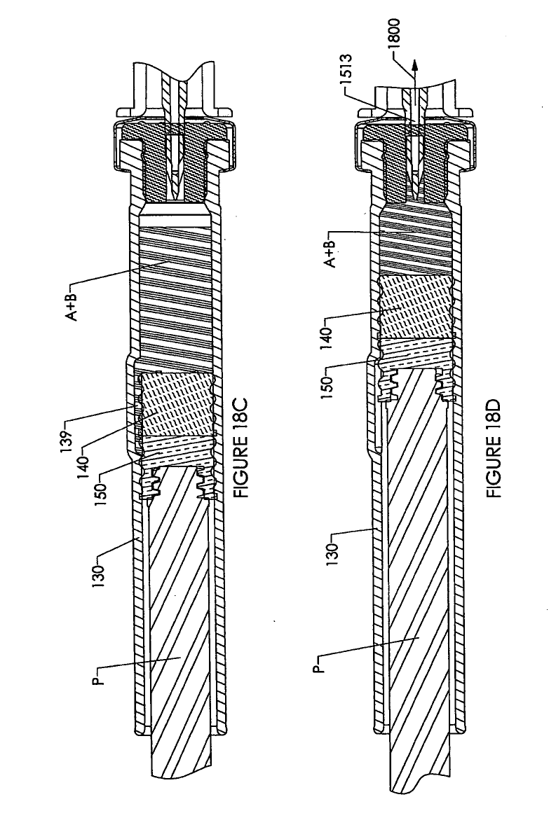

Cartridge 130 has a bypass recess 139 in wall 131, forming a longitudinally oriented radially extending groove of a slightly greater length than the length of central piston 140 (i.e. greater than the distance between distal surface 143 and proximal surface 145). When central piston 140 is aligned with bypass recess 139 such that the length of bypass recess 139 extends beyond distal surface 143 at one end and proximal surface 145 at the other end, a fluid bypass passage is formed, by which fluid can travel from the first chamber to the second chamber around central piston 145 via bypass recess 139. In this bypass position, central piston 140 is frictionally engaged by the inner surface of wall 131 so as to resist further axial movement unless it is subject to a further activating force, such as from actuating piston 150. FIGS. 18A to 18D illustrate the process of mixing of constituents A and B through bypass recess 139 in further detail.

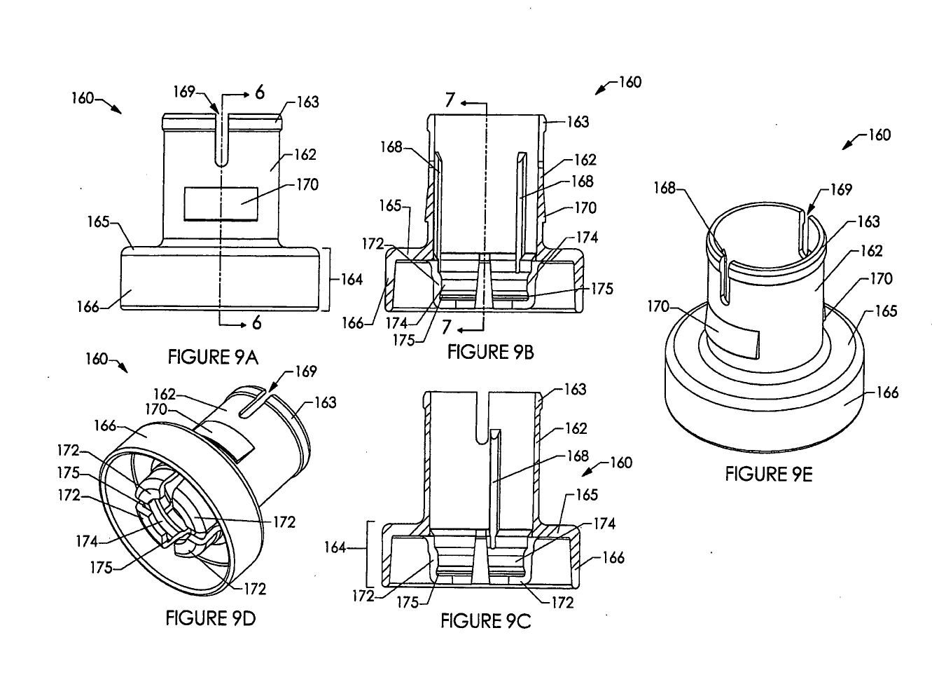

Activation cap 160 (shown in further detail in FIGS. 9A to 9E) is arranged to receive the proximal end of cartridge 130 in base grips 172 in a base portion 164 of the activation cap 160. Base grips 172 are somewhat L-shaped in cross-section and extend proximally of flange 165 and radially inwardly of the end rim 138 of the cartridge. Base grips 172 thus resemble circumferentially spaced gripping fingers.

Activation cap 160 has a sleeve 162 extending longitudinally and distally from base portion 164. Sleeve 162 has a protruding rim 163 disposed around a distal end of activation cap 164 for engaging the groove formed between internal ribs 119 of syringe socket 110, when delivery device 100 is in its pre-activated position. Thus, activation cap 160 serves to assist in locating cartridge 130 within syringe socket 110 by holding cartridge 130 in a fixed longitudinal position with respect to syringe socket 110 prior to activation of delivery device 100.

Activation cap 160 further comprises longitudinal alignment ribs 168 arranged to be slightly spaced from the outer surface of wall 131 to maintain the proper longitudinal alignment of cartridge 130 within the activation cap 160. Three such alignment ribs 168 are provided in activation cap 160 but more may be provided, if desired. Preferably, alignment ribs 168 are equally spaced around the inner circumference of sleeve 162.

Base grips 172 also serve to maintain proper longitudinal alignment of the cartridge 130 within activation cap 160 by gripping an end rim 138 of the cartridge 130 in a firm, yet resiliently displaceable, manner. Base grips 172 surround end rim 138 but define a base opening 173 (of a smaller diameter than the internal diameter of the cartridge at its proximal end) through which a plunger can be inserted to engage with actuating piston 150. Base grips 172 include grip recesses 175 for receiving end rim 138 and grip protrusions 174 for overlying wall 131 of cartridge 130 immediately adjacent end rim 138 and thereby providing a snap fit of the end rim 138 in base grips 172.

The base opening 173 defined by base grips 172 is sized so as to allow a plunger to be received therethrough for connection or contact with the actuating piston 150. According to preferred plunger embodiments (such as are described later), the outer surface of the plunger and base grips 172 interfere with each other during progression of the plunger, at least in its first stroke in which actuating piston 150 is pushed distally to mix constituents A and B. This interference of the plunger with base grips 172 serves to retard movement of the plunger as it progresses in its first stroke and thus mitigates against an overly vigorous actuation of the plunger during mixing of the constituents.

Because of the outwardly deflectable resilience of base grips 172, interference of an inwardly directed face of each of the base grips 172 with the outer surface of the plunger results in a slight radially outward deflection of base grips 172. Base grips 172 are formed to have a shape memory which biases them back to an inward rest position when they are deflected outwardly, thus engaging the inner face of each of the base grips 172 in contact with the outer surface of the plunger in a frictional manner to resist relative movement therebetween. The dimensions of the outer surface of the plunger and the inward extension of base grips 172 may be sized to provide greater or lesser friction in general or along certain parts of the plunger length corresponding to certain stages of device actuation.

Activation cap 160 also has a flange 165 extending radially of sleeve 162 and forming part of the base portion 164. An apron 166 extends proximally and longitudinally from flange 165 so as to extend slightly proximally of base grips 172 and provide an annular support for the cartridge 130 when stood upright.

In order for cartridge 130 to be powder filled, it is desirable for cartridge 130 to be carried upright in a stable manner, for example along a conveyor, before stopper 133 is inserted in the neck 132. When cartridge 130 is received in activation cap 160, it can be carried upright in a significantly more stable manner than if it rested on end rim 138. Thus, the structure of base portion 164 of the activation cap 160 is advantageous, having regard to the added stability provided by the annular footprint of apron 166 to cartridge 130 during carriage thereof in an upright position as part of a cartridge assembly in a pre-filling process prior to insertion of cartridge 130 and activation cap 160 in syringe socket 110.

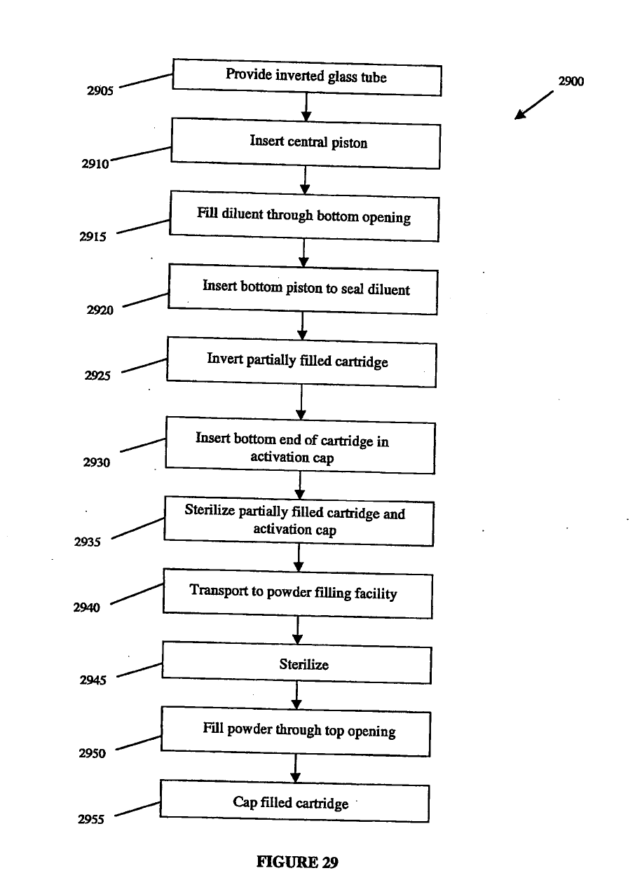

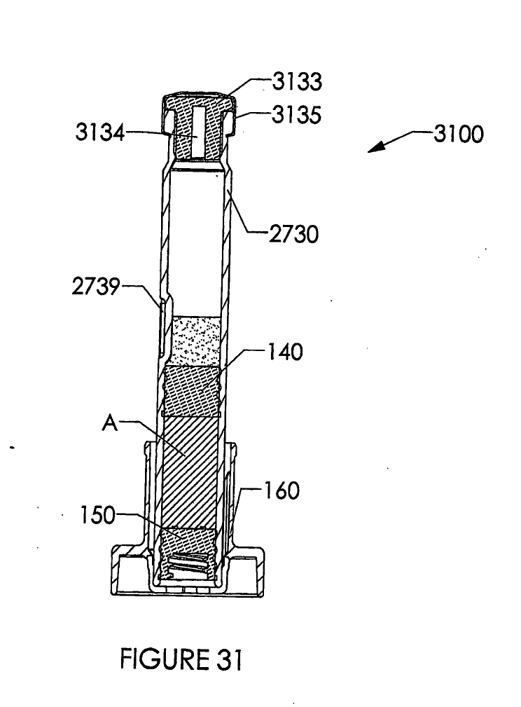

This advantageous structure of activation cap 160 is also useful with other forms of double-chambered cartridge, for example such as is shown and described in relation to FIG. 31. Use of activation cap 160 in a method of cartridge assembly is shown and described in relation to FIGS. 29 to 31.

Activation cap 160 further includes locking sleeve protrusions 170 on an external wall of sleeve 162 for engaging internal ribs 119 in a locking manner when delivery device 100 is in its activated position (as shown in FIG. 1E) so that the activation cap is difficult to manually withdraw from syringe socket 110. Locking sleeve protrusions 170 are angled outward protrusions sloping outwardly in a proximal direction and configured to have a proximal shoulder thereof received within the groove defined between internal ribs 119 of syringe socket 110 when delivery device 100 is in its activated position.

Activation cap 160 further includes two longitudinal sleeve recesses 169 disposed diametrically opposite each other in a distal portion of sleeve 162. Longitudinal sleeve recesses 169 allow protruding rim 163 at the distal extremity of activation cap 160 to have a degree of resilient inward deflection when activation cap 160 is inserted partially within the proximal opening of syringe socket 110, as shown in FIGS. 1A and 1B. This resilient deflection allows protruding rim 163 to be more easily pushed past the proximal internal rib 119 for receipt within the circumferential groove defined between the internal ribs 119.

Activation cap 160 and syringe socket 110 are preferably formed of a polycarbonate material, for example such as Dow Calibre 2061, so as to be substantially transparent. Alternatively, they may be formed of ABS (acrylonitirle-butadiene-styrene copolymers) or K-Resin® (styrene butadiene copolymer). Wall 131 of cartridge 130 is preferably formed of type 1 borosilicate glass, optionally containing cerium oxide, which is suitable for use in gamma ray sterilization. Alternatively, suitable plastics or other suitable materials may be used to form wall 131. Such alternative materials should be amenable to normal sterilization procedures and be moldable in the appropriate shapes.

FIGS. 1C and 1D show delivery device 100 in exploded perspective and side-sectional views, respectively. As shown in these figures, a luer cap 183 may be placed over male luer fitting 112 to close off fluid passage 113 until needle 180 is fitted onto male luer fitting 112. Once delivery device 100 is in its activated position and is ready for fluid delivery through needle 180, protective cap 184 is removed from needle 180, the needle 180 is attached to male luer fitting 112 and the fluid may be delivered therethrough, via needle fluid channel 181.

While FIGS. 1A and 1B show the delivery device 100 in its pre-activated position, FIG. 1E shows the delivery device 100 in the activated position. Referring particularly to FIG. 1E, in the activated position, fluid delivery device 100 is arranged such that cartridge 130 is pressed in a distal direction within syringe socket 110 by bringing flange 118 and activation cap 160 towards each other such that spike 114 is forced to pass through cartridge cap opening 136 and puncture a central (thin) portion of stopper 133 and enter stopper cavity 134, thus creating a fluid connection between the second chamber of cartridge 130 and the needle fluid channel 181 via fluid passage 113.

Flange 118 and activation cap base portion 164 are configured such that, in the activated position, flange 165 fits within an annular depression in the proximal side of flange 118 in a mating manner. In the activated position, flange 118 overlies, and radially extends beyond, flange 165.

In the activated position, fluid delivery device 100 is ready to have actuating piston 150 moved distally within cartridge 130 to begin mixing constituents A and B.

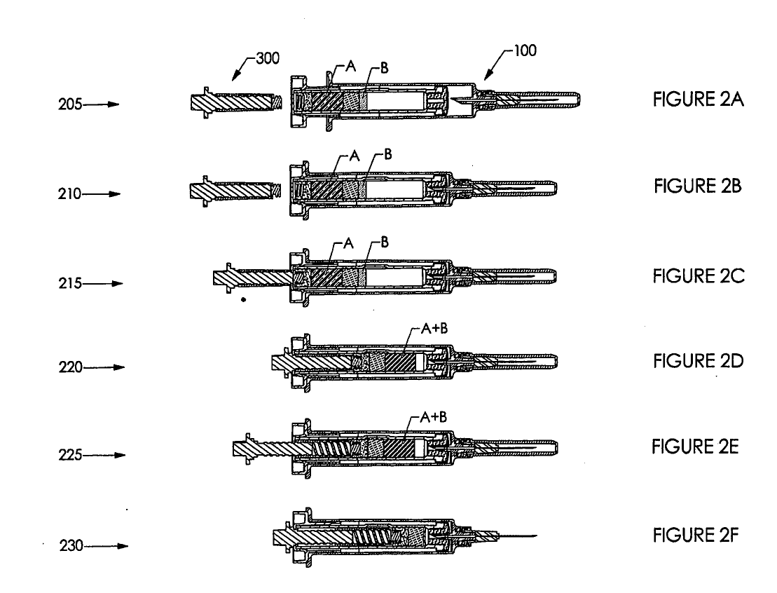

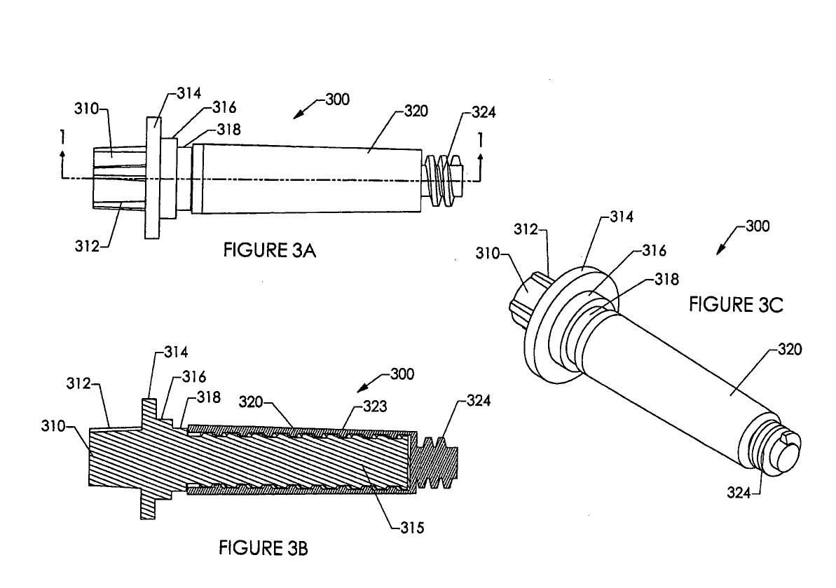

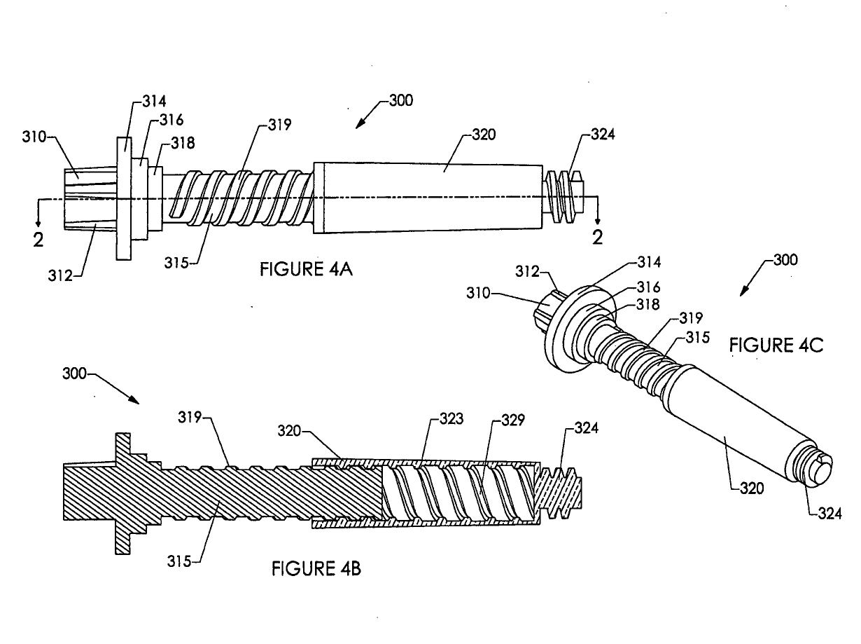

FIGS. 2A to 2F show respective stages of delivery device 100 during a process of use thereof. FIG. 2A shows a first step 205, in which the delivery device 100 is provided, including an extensible plunger 300 (shown and described in further detail in relation to FIGS. 3A to 3C and 4A to 4C) and having constituents A and B located within the first and second chambers of cartridge 130. The delivery device 100 shown in FIG. 2A is in its pre-activated position, corresponding to that shown in FIGS. 1A and 1B.

In FIG. 2B, step 210 shows delivery device 100 having been placed in its activated position (corresponding to FIG. 1E), by pushing flange 118 and activation cap 160 together, thereby puncturing stopper 133 with spike 114. In FIG. 2C, step 215 shows attachment of plunger 300 to actuating piston 150 by engagement of a male threaded end 324 of plunger 300 with plunger socket 154, without actuation of actuating piston 150 by plunger 300.

In FIG. 2D, step 220 shows actuation of actuating piston 150 in a first stroke by insertion of plunger 300 into cartridge 130 in a distal direction, thereby placing a central piston 140 in a bypass position and allowing fluid flow of constituent A into the second chamber to mix with constituent B. Plunger 300 is prevented from extending any further into cartridge 130 by abutment of an outer shoulder 316 of plunger 300 against a proximal surface of base grips 172. In an alternative method, steps 215 and 220 may be performed before step 210. Thus, delivery device 100 may be activated after mixing constituents A and B.

In FIG. 2E, step 225 shows extensible plunger 300 in an extended position, in which a sleeve thereof remains received within wall 131 of cartridge 130, while a shaft 315 of the plunger 300 is extended proximally relative to the sleeve 320, thereby effectively lengthening the plunger 300 and allowing for a further stroke of the plunger 300 within cartridge 130.

In FIG. 2F, step 230 shows plunger 300 actuated in a further stroke, with needle protective cap 184 removed, for effecting delivery of the mixed pharmaceutical constituents A and B through needle fluid channel 181 via syringe socket fluid channel 113.

Referring now to FIGS. 3A to 3C, there is shown an extensible plunger 300 in a retracted position. Referring also to FIGS. 4A to 4C, the extensible plunger 300 is shown in its extended position. Plunger 300 is arranged to extend and retract by means of screw-threaded engagement of a sleeve 320 with a shaft 315. In the retracted position, shaft 315 and sleeve 320 are substantially mated with each other. In the extended position, sleeve 320 extends substantially distally away from shaft 315.

Plunger 300 has a head portion 310 with longitudinally oriented ribs 312 thereon for facilitating gripping of the head 310 by fingers of a hand while screwing the shaft 315 in or out of sleeve 320. A flange 314 is provided distally of head portion 310 for facilitating driving of the plunger by allowing the user of delivery device 100 to push against flange 314 during actuation of actuating piston 150.

Plunger 300 also has an outer shoulder 316 of lesser diameter than flange 314 and located distally thereof. As described in relation to FIG. 2D, at the end of the first stroke of plunger 300, outer shoulder 316 abuts the proximal face of base grips 172 at its distal face for preventing plunger 300 from driving too far within cartridge 130 in its first stroke.

An inner shoulder 318 is also provided distally of outer shoulder 316 and having a smaller diameter. Inner shoulder 318 has a distal surface for abutting, and limiting the further proximal progress of, the proximal end of sleeve 320 when the plunger 300 is in its retracted position.

Shaft 315 extends distally of inner shoulder 318 and has male thread 319 thereon for mating with female thread 329 on the inside of sleeve 320.

Sleeve 320 has a male threaded end 324 at its distal extremity for screw threaded engagement thereof with actuating piston 150 within plunger socket 154. Sleeve 320 is substantially hollow and tubular proximally of male threaded end 324, although female thread 329 is provided on the internal wall of sleeve 324 for screw-threaded engagement with the male thread 319 around shaft 315.

An outer surface 323 of sleeve 320 is tapered from the proximal end thereof toward the distal end thereof. The degree of inward taper from the proximal end of sleeve 320 to the distal end of outer surface 323 is between about 0.5 degrees to 2 degrees, and preferably about 1 degree. This taper is only provided on the external surface of sleeve 320, not internally.

The purpose of the taper is to create an increasingly tight interference fit of the outer surface 323 of sleeve 320 with the inwardly facing surface of the grips 172 around base opening 173 as plunger 300 is driven distally in its first stroke, thus slowing or resisting distal movement of actuating piston 150 within cartridge 130 as plunger 300 nears the end of its first stroke. This slowing or movement resistance increases with the progress of plunger 300 during distal movement within its first stroke, thus mitigating against an overly vigorous actuation of plunger 300.

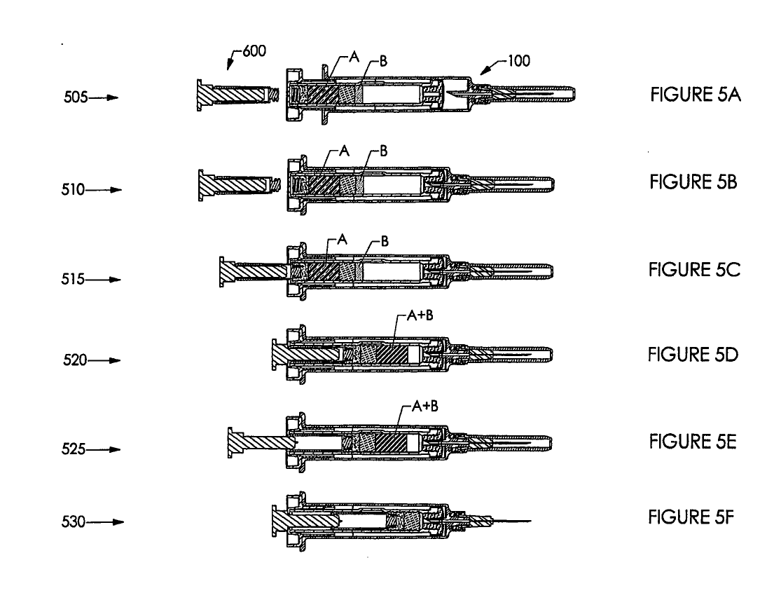

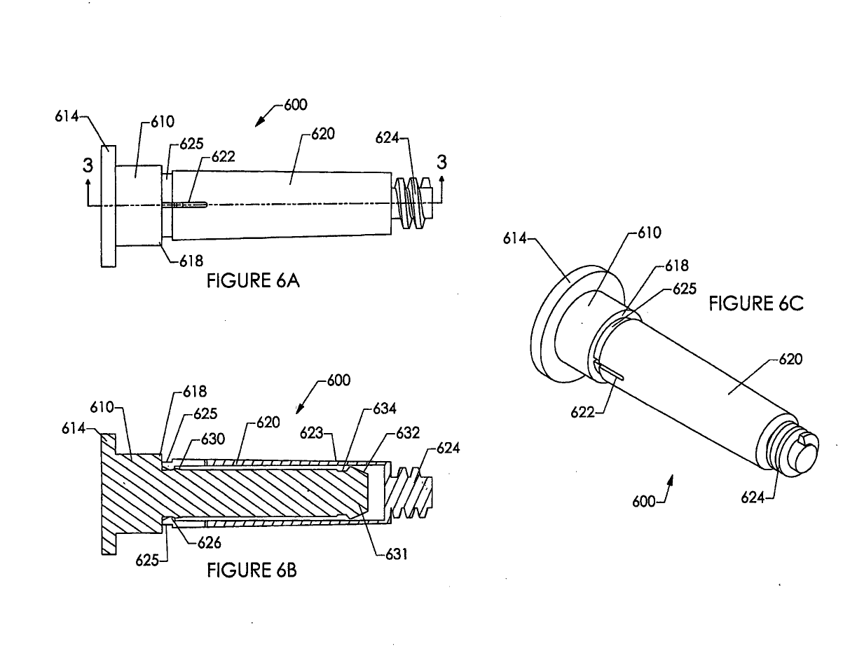

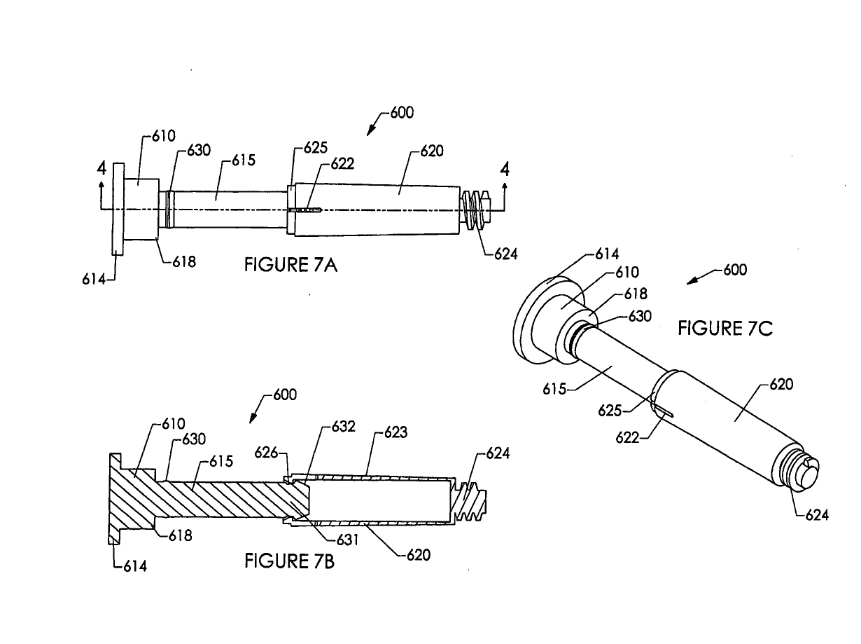

FIGS. 5A to 5F show respective stages of delivery device 100 during use thereof. FIG. 5A shows a first step 505, in which the delivery device 100 is provided, including an extensible plunger 600 (shown and described in further detail in relation to FIGS. 6A to 6C and 7A to 7C) and having constituents A and B located within the first and second chambers of cartridge 130. The delivery device 100 shown in FIG. 5A is in its pre-activated position, corresponding to that shown in FIGS. 1A and 1B.

In FIG. 5B, step 510 shows delivery device 100 having been placed in its activated position, by pushing flange 118 and activation cap 160 together, thereby puncturing stopper 133 with spike 114. In FIG. 5C, step 515 shows attachment of plunger 600 to actuating piston 150 by engagement of a male threaded end of 624 of plunger 600 with plunger socket 154, without actuation of actuating piston 150 by plunger 600.

In FIG. 5D, step 520 shows actuation of actuating piston 150 in a first stroke by insertion of plunger 600 into cartridge 130 in a distal direction, thereby placing a central piston 140 in a bypass position and allowing fluid flow of constituent A into the second chamber to mix with constituent B. Plunger 600 is prevented from extending any further into cartridge 130 by abutment of a shoulder 618 of plunger 600 against a proximal surface of base grips 172. In an alternative method, steps 515 and 520 may be performed before step 510. Thus, delivery device 100 may be activated after mixing constituents A and B.

In FIG. 5E, step 525 shows extensible plunger 600 in an extended position, in which a sleeve thereof remains received within wall 131 of cartridge 130, while a shaft 615 of the plunger 600 is extended proximally relative to the sleeve 620, thereby effectively lengthening the plunger 600 and allowing for a further stroke of the plunger 600 within cartridge 130.

In FIG. 5F, step 530 shows plunger 600 actuated in a further stroke, with needle protective cap 184 removed, for effecting delivery of the mixed pharmaceutical constituents A and B through needle fluid channel 181 via syringe socket fluid channel 113.

Referring now to FIGS. 6A to 6C, there is shown an alternative plunger 600 in its retracted position. Referring also to FIGS. 7A to 7C there is shown the alternative plunger 600 in its extended position.

Plunger 600 is similar in operation and function to plunger 300, except that sleeve 620 and shaft 615 are moveable relative to each other in a sliding manner, rather than in a screw-threaded manner. Sleeve 620 has an outer surface 623 tapered in a similar manner to sleeve 320 and for a similar purpose. Also, sleeve 620 has a male threaded end 624 similar to male threaded end 324 for engaging with actuating piston 150.

Plunger 600 has a head portion 610 disposed generally proximally, with a flange 614 extending radially at the proximal extremity of head portion 610. Flange 614 may be used to facilitate actuation of plunger 600 by pushing thereon by fingers of a hand. Head portion 610 has a shoulder 618 disposed distally of flange 614 and having a distally facing surface against which the proximal part of sleeve 620 abuts in the retracted position.

Shaft 615 has a shaft base protrusion 630 protruding outwardly near the base of the shaft 615 around its circumference for cooperating with internal latching protrusions 626 to retain sleeve 620 in its retracted position. Upon the application of sufficient force tending to longitudinally separate the shaft 615 and head portion 610 from sleeve 620, internal latching protrusions 626 are resiliently deflected outwards to pass across shaft base protrusion 630, after which internal latching protrusions 626 freely slide across most of the length of shaft 615.

In its extended position, plunger 600 has its sleeve 620 and shaft 615 arranged such that internal latching protrusions 626 are received within a shaft head recess 634 extending circumferentially around shaft 615 at shaft head 631. Internal latching protrusions 626 are formed on the inside of inwardly stepped portions 625 so as to narrow the proximal opening of sleeve 620 and bias internal latching protrusions 626 to clamp inwardly on shaft 615. Accordingly, when internal latching protrusions 626 are received within shaft head recess 634, they are biased to latch into the recess 634 and resist further relative longitudinal movement between shaft 615 and sleeve 620.

Shaft head 631 is shaped so as to have a portion of greater diameter than the rest of shaft 615 immediately distally of shaft head recess 634 for preventing internal latching protrusions 626 from sliding over the end of shaft head 631 and thus preventing the separation of sleeve 620 from shaft 615. Immediately distally of the widened diameter portion of shaft head 631, a sloped shaft head surface 632 extends toward the distal extremity of shaft head 631.

Sloped shaft head surface 632 is generally frustoconical and extends only as far as a flat distal end face of shaft 615. Sloped shaft head surface 632 is provided for facilitating easy initial insertion of shaft 615 within sleeve 620 as inwardly stepped portions 625 are forced to widen as they pass across sloped shaft head surface 632 toward head portion 610. Inwardly stepped portions 625 have a mating internal frustoconical surface adjacent the proximal opening of sleeve 620 for cooperating with sloped shaft head surface 632 during assembly of plunger 600 by insertion of shaft 615 within sleeve 620.

Two longitudinal recesses (or notches or slots) 622 extend distally from the proximal opening of sleeve 620 and diametrically opposite one another. Longitudinal recesses 622 allow inwardly stepped portions 625 and internal latching protrusions 626 to resiliently outwardly deflect as they pass over the widened portion of shaft head 631 and shaft base protrusions 630. More than two recesses 622 may be provided, if desired.

Plunger 600 is extensible between retracted and extended positions for lengthening plunger 600 after an initial stroke within the delivery device 100, following which the plunger 600 can be extended to drive actuating piston 150 and central piston 140 in a final stroke to deliver the mixed substance from delivery device 100.

Both plungers 300 and 600 are preferably formed of polystyrene. Alternatively, they may be formed of ABS (acrylonitrile-butadiene-styrene copolymers) or polycarbonate.

In alternative embodiments of plungers 300 and 600, the tapered outer surface of the sleeve 320 or 620 may be corrugated or ridged so as to provide further interference between the outer surface of the sleeve and the inwardly facing surface of grips 172 of base portion 164 as the plunger 300 or 600 is driven distally in its first stroke. These corrugations or ridges thus serve to slow or resist distal movement of the plunger in its first stroke and thus mitigate against an overly vigorous actuation of the plunger 300 or 600.

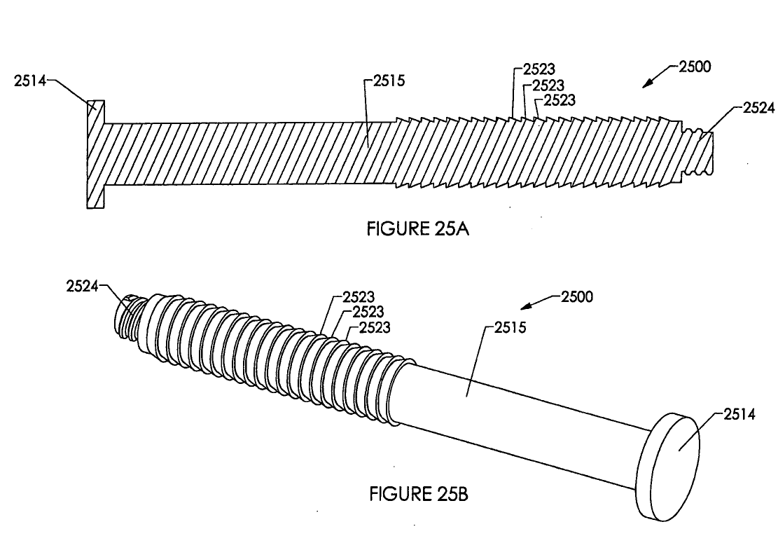

Further alternative plunger embodiments may employ a corrugated or ridged outer surface as well as, or in addition to, the tapered outer surface along a single inextensible shaft. While such plungers do not have the advantageous two-step plunger actuation method of plungers 300 and 600, they would nevertheless have the advantageous corrugations or ridges and/or tapered outer surface, serving to retard or resist movement of the plunger during the mixing of constituents A and B and thus mitigate against an overly vigorous actuation of the plunger. An example of an inextensible plunger having a ridged outer surface is shown and described in relation to FIGS. 25A and 25B.

The plunger embodiments shown and described in relation to the drawings employ a screw-threaded piston engaging portion on a distal end thereof, and actuating piston 150 has been shown throughout the drawings and described as having a female threaded plunger socket for mating with the male threaded piston engaging portion of the plunger. However, where the plunger is only required to be actuated in a distal direction, such screw-threaded engagement of the plunger with actuating piston 150 is not necessary. Accordingly, alternative embodiments of the invention may employ a plunger having a piston engaging portion without male threads and an actuating piston without a female threaded socket. Such embodiments rely on contact of a distal end face of the plunger with the proximal face of actuating piston 150 during actuation of the plunger and actuating piston.

Syringe socket 110 further comprises a plurality of cap alignment bosses 123 arranged adjacent the proximal opening of syringe socket 110, as shown in FIGS. 8B and 8C. Cap alignment bosses 123 are formed as discrete protrusions located at a portion of syringe socket 110 where tubular wall 116 curves radially outwardly to form flange 118. Because of this curvature of wall 116 adjacent the proximal opening of syringe socket 110, an activation cap, such as activation cap 160 or 1560 (shown in FIG. 15A), may have a tendency to be slightly cocked as the sleeve 162 is received within the proximal opening of the syringe socket 110 in the pre-activated position.

Cap alignment bosses 123 serve to assist correct axial alignment of activation cap 160 within syringe socket 110 in the pre-activated position by mitigating against cocking of the activation cap 160. This is done by providing cap alignment bosses 123 with a short longitudinally aligned, inwardly directed face arranged to longitudinally align with the inner surface of wall 116 of syringe socket 110 in its untapered portion.

Cap alignment bosses 123 are arranged around the proximal opening of sleeve 110 so as to guide sleeve 162 of activation cap 160 as it enters the proximal opening and protruding rim 163 is received between internal ribs 119. The inwardly directed faces of the cap alignment bosses 123 are arranged to contact the sleeve 162 of activation cap 160 if it tends to cock and to limit the degree of such cocking.

As shown in FIGS. 8B and 8C, six cap alignment bosses 123 may be arranged around the proximal opening of syringe socket 110. However, as few as three cap alignment bosses 123 may be so provided, or more such bosses may be provided, providing they serve the function of mitigating against cocking of the activation cap 160 in the pre-activated position. Preferably, cap alignment bosses 123 are equally spaced around the circumference of the proximal opening of syringe socket 110.

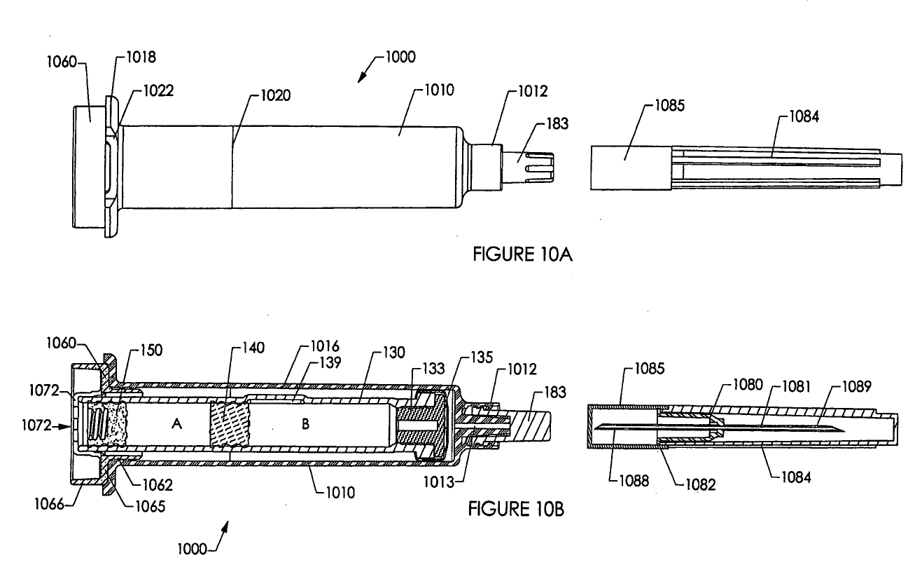

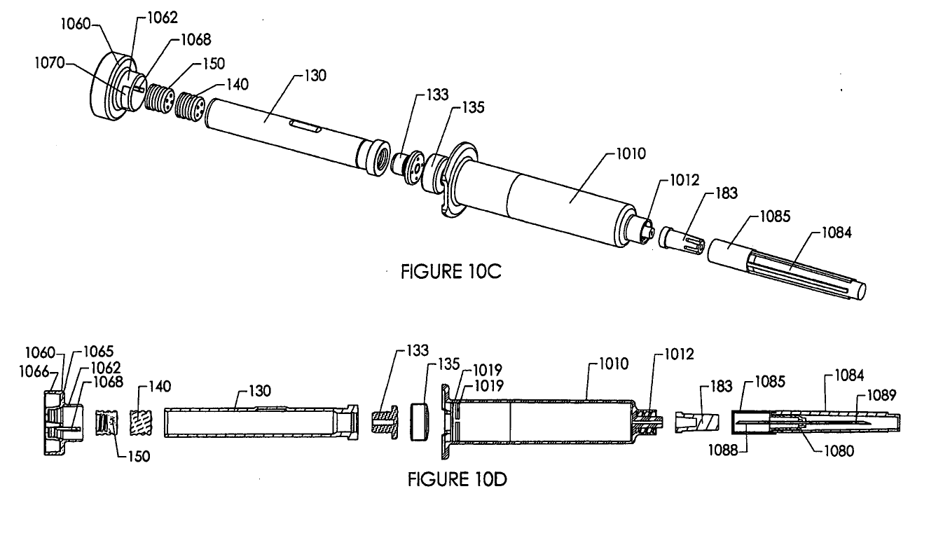

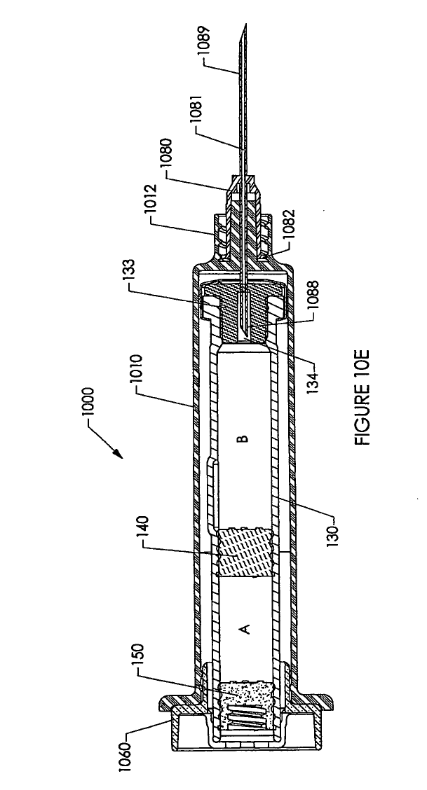

Referring now to FIGS. 10A to 10E, there is shown a delivery device 1000 according to a further embodiment. Delivery device 1000 is similar to delivery device 100 in many respects. Accordingly, rather than repeating the same detailed description provided in relation to delivery device 100 (with reference to FIGS. 1A to 9E), differences between the embodiments will be described instead. A similar approach is taken to describing further embodiments below.

As shown in FIGS. 10A and 10B, in which delivery device 1000 is shown in a pre-activated position, syringe socket 1010 does not have a spike extending proximally within wall 1016 at the fluid delivery end. Further, activation cap 1060 has a sleeve 1062 of shorter length than sleeve 162 and is fully received within the proximal end opening of syringe socket 1010. These different features of delivery device 1000 are related to the method of activation employed by this embodiment. Specifically, delivery device 1000 is placed in its activated position by insertion of a proximal end 1088 of a double-ended needle 1080 through fluid passage 1013 so as to penetrate stopper 133. Delivery device 1000 is shown in its activated state in FIG. 10E.

Because of the use of a double-ended needle 1080 in this embodiment, to activate the delivery device (prior to mixing of the constituents) it is not necessary to move cartridge 130 distally within syringe socket 1010 as part of the activation step, and thus activation cap 1060 has a shorter sleeve 1062.

Delivery device 1000 employs the same cartridge 130 as delivery device 100. Other features of delivery device 1000 are similar to those described in relation to delivery device 100. For example, syringe socket 1010 has a tapered transition point 1020 along tubular wall 1016, which serves as the transition point between the non-tapered portion located proximally thereof and the tapered portion located distally thereof. Syringe socket 1010 also comprises a flange 1018 and flattened portion 1022 and male luer fitting 1012 similar to the corresponding features of delivery device 100. Further, internal ribs 1019 located internally adjacent the proximal opening of syringe socket 1010 correspond to internal ribs 119 of syringe socket 110.

Shortened activation cap 1060 is, apart from having a shorter sleeve 1062 than activation cap 160, substantially similar. For example, activation cap 1060 has a flange 1065 and apron 1066 extending proximally therefrom, as well as longitudinal alignment ribs 1068 disposed along the internal wall of sleeve 1062. Activation cap 1060 does not have a protruding rim at the distal end of sleeve 1062 as it is unnecessary.

Locking sleeve protrusions 1070 are provided on sleeve 1062 for fitting a proximal shoulder thereof between internal ribs 1019 to retain activation cap 1060 within the proximal end of syringe socket 1010 without appreciable relative axial movement therebetween.

Referring specifically to FIG. 10E, delivery device 1000 is shown in its activated state, with female luer lugs 1082 of double-ended needle 1080 engaged in male luer fitting 1012, such that a proximal end 1088 of needle 1080 extends through fluid passage 1013 and pierces the thin central portion of stopper 133. Thus, fluid communication is established between the second chamber of cartridge 130 and a needle fluid channel 1081 of needle 1080 for delivering the contents of the second chamber to a patient when a distal end 1089 of needle 1080 is inserted in the patient.

As part of the activation method applicable to delivery device 1000, luer cap 183 is removed (if present) from male luer fitting 1012, a proximal protective cap 1085 is removed from needle 1080 to expose proximal end 1088, and needle 1080 is then fitted on to male luer fitting 1012 so as to insert proximal end 1088 through fluid passage 1013. Distal protective cap 1084 may be retained over the distal end 1089 of needle 1080 until immediately prior to injection. Apart from the noted differences in activation, delivery device 1000 may be used as shown and described in relation to FIGS. 2A to 2F or 5A to 5F.

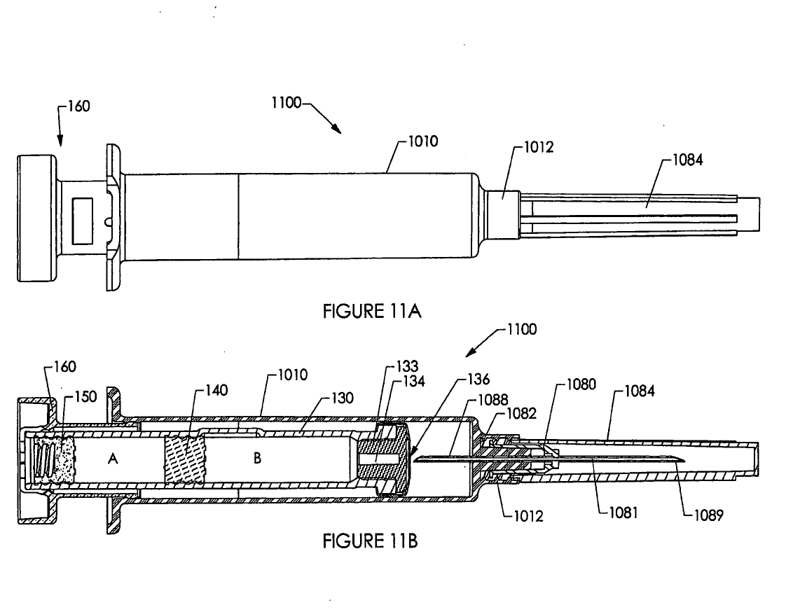

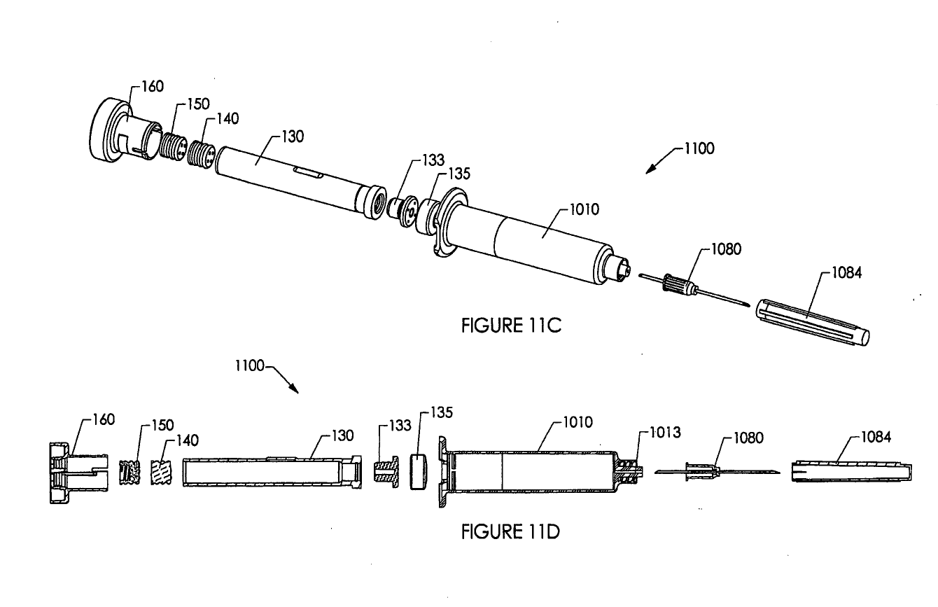

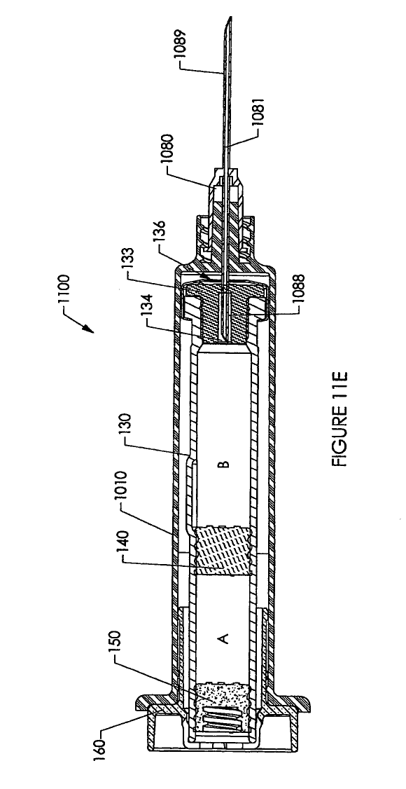

Referring now to FIGS. 11A to 11E, there is shown a delivery device 1100 according to a further embodiment. Delivery device 1100 is similar to the above-described embodiments of delivery devices 100, 1000, in that it uses syringe socket 1010 and needle 1080 with cartridge 130, but uses activation cap 160 instead of activation cap 1060.

FIGS. 11A and 11B show delivery device 1100 in a pre-activated position, where activation cap 160 (having a longer sleeve 162 than that of activation cap 1060) holds the distal end of cartridge 130 away from fluid passage 1013, while proximal end 1088 of needle 1080 extends therethrough. Thus, delivery device 1100 resembles delivery device 100, except that spike 114 is replaced by the proximal end 1088 of needle 1080, which is not integrally formed with the syringe socket (whereas spike 114 is preferably integrally formed with the distal end of syringe socket 110).

Delivery device 1100 is placed in the activated state in the same way as delivery device 100, that is, by pushing flange 1018 and base portion 164 of activation cap 160 towards each other so as to cause stopper 133 to be driven on to the proximal end 1088 of needle 1080. The activated state of delivery device 1100 is shown in FIG. 11E, where the proximal end 1088 of needle 1080 is shown extending within stopper cavity 134, thus establishing a fluid communication from the second chamber of cartridge 130 through needle fluid channel 1081 for injection of the mixed constituents through distal end 1089 of needle 1080. Delivery device 1100 may be used as shown and described in relation to FIGS. 2A to 2F or 5A to 5F.

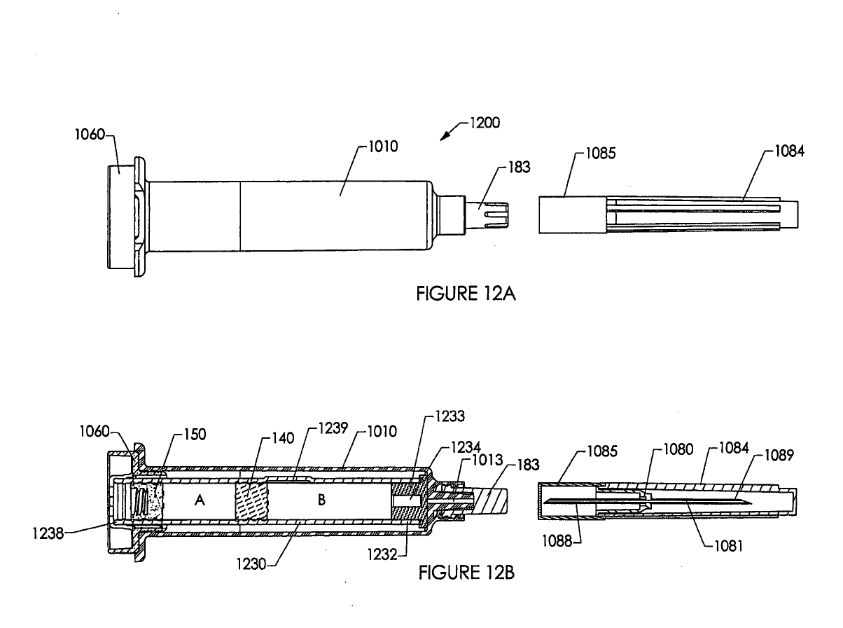

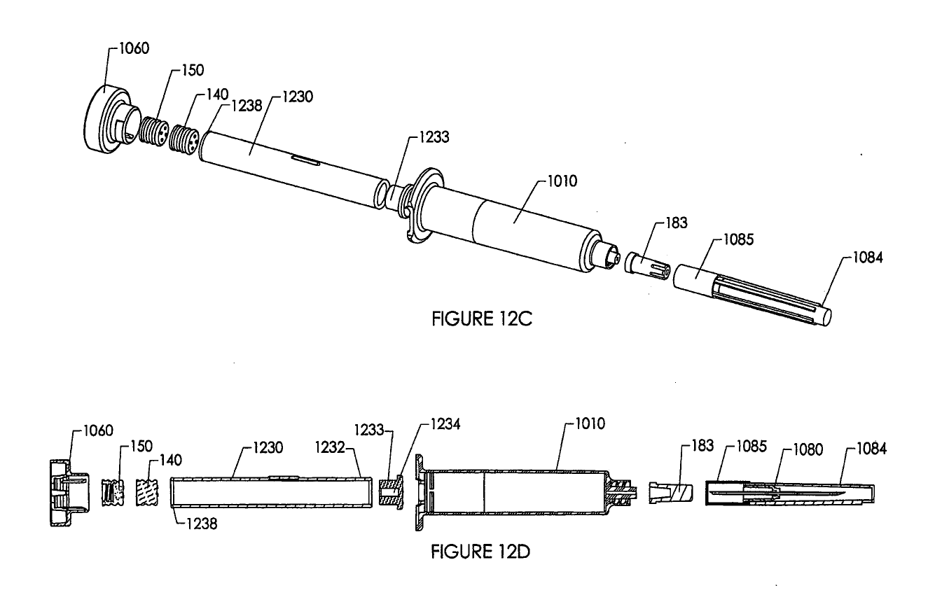

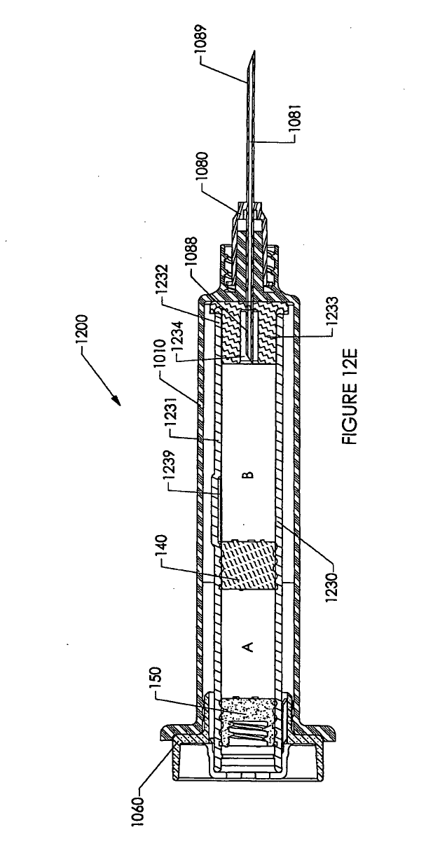

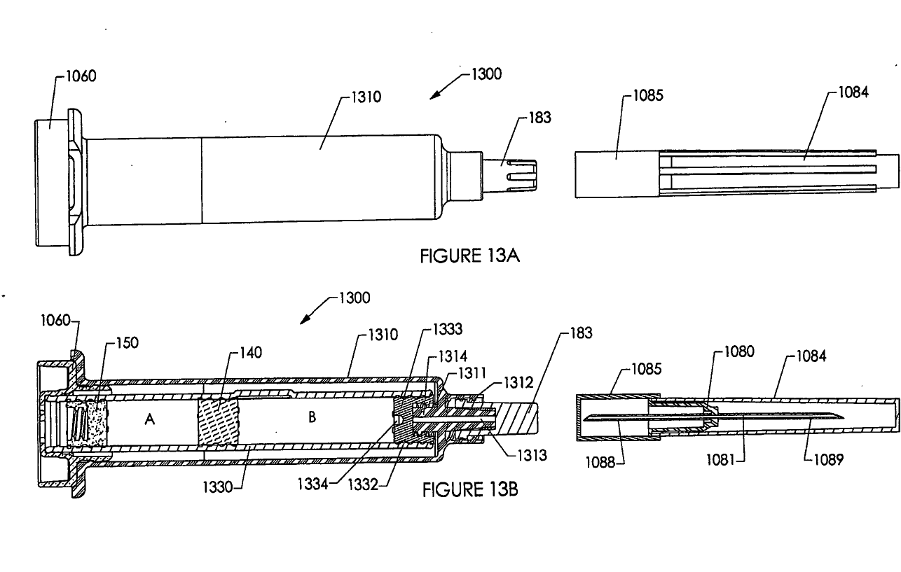

Referring now to FIGS. 12A to 12E, there is shown a delivery device 1200 according to yet another embodiment. Delivery device 1200 is identical to delivery device 1000, except that it uses a modified cartridge 1230 having a straight neck portion 1232 and a slightly larger stopper 1233. Further, cartridge 1230 does not employ a cartridge cap as neck portion 1232 is straight and does not have a lip over which the cap could clamp.

Delivery device 1200 is shown in FIGS. 12A and 12B in its pre-activated state, in which stopper 1233 abuts or is adjacent to an internal end wall of syringe socket 1010 so that stopper cavity 1234 is axially aligned with fluid passage 1013 within male luer fitting 1012. Delivery device 1200 is activated in a similar way to delivery device 1000, whereby proximal end 1088 of needle 1080 is inserted through fluid passage 1013 so as to puncture a thin central portion of stopper 1233 and extend into stopper cavity 1234. Delivery device 1200 is shown in this activated position in FIG. 12E.

Cartridge 1230 is similar to cartridge 130, except for its modified neck 1232, stopper 1233 and lack of a stopper cap. Neck portion 1232 is slightly more elongated than neck 132 of cartridge 130, in order to press stopper 1233 against the internal distal end wall of syringe socket 1010. Stopper 1233 has a radially extending flange portion extending radially beyond the outer wall of cartridge 1230 so that wall 1231 can press against the flange of stopper 1233 adjacent a region of the stopper where the flange joins its cylindrical body portion.

Activation cap 1060 holds the proximal end of cartridge 1230 in an axially aligned manner within syringe socket 1010 so that stopper 1233 has its cavity 1234 substantially aligned with fluid passage 1013, thus allowing proximal end 1088 of needle 1080 to pass through fluid passage 1013 into stopper cavity 1234. Delivery device 1200 may be used, apart from the different activation step as noted, as shown and described in relation to FIGS. 2A to 2F or 5A to 5F.

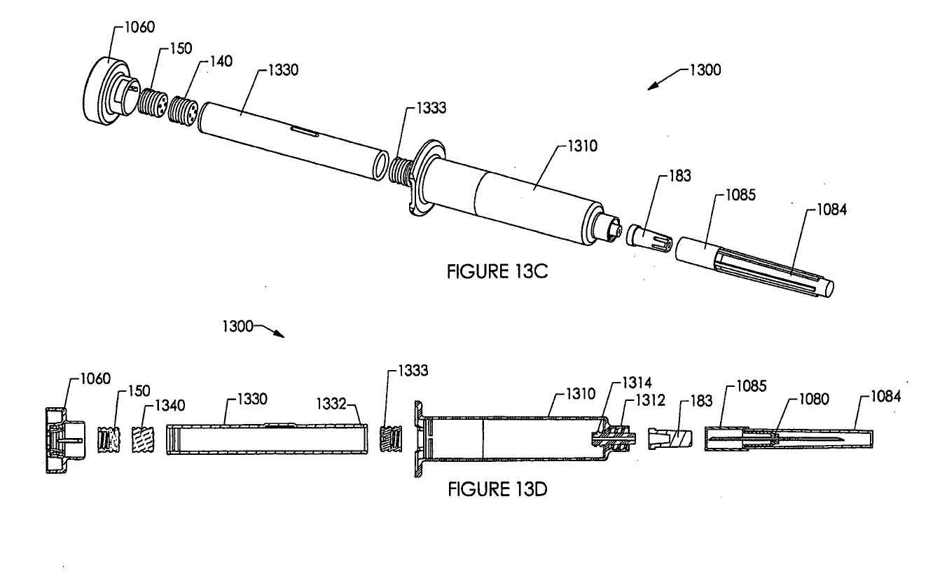

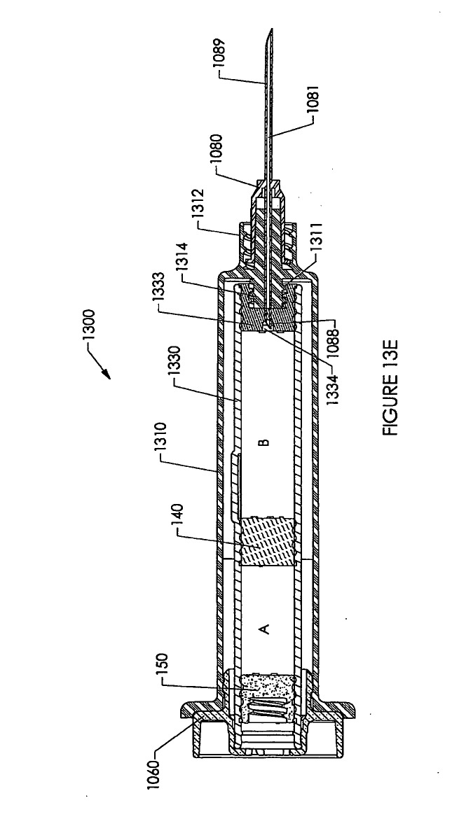

Referring now to FIGS. 13A to 13E, there is shown a delivery device 1300 according to yet another embodiment. Delivery device 1300 is similar to delivery device 1200, except that it uses a modified stopper 1333 and a syringe socket 1310 having a modified fluid delivery end.

Delivery device 1300 uses a cartridge 1330 similar to cartridge 1230, except that it uses modified stopper 1333. Like cartridge 1230, cartridge 1330 uses a straight neck 1332 extending nearly to the inner end wall of syringe socket 1310 at its fluid delivery end. Stopper 1333 is formed as a piston of similar shape and dimension to actuating piston 150. Stopper 1330 has a screw-threaded recess in its distal face, similar to plunger socket 154 of actuating piston 150, for engaging with a male threaded portion 1314 extending centrally in a proximal direction from the inner end wall of syringe socket 1310 at its fluid delivery end.

Both cartridges 1230 and 1300 advantageously have a larger internal diameter at the neck and a smaller outer diameter than a standard cartridge neck, such as is used in cartridge 130. The standard cartridge neck employed by cartridge 130 has a largest outer diameter (around the flange) of about 13 millimeters and a smallest internal diameter of about 7.05 millimeters. This internal diameter is, according to current manufacturing practice, the smallest neck opening that can be used for powder filling of the cartridge, without a substantial reduction in filling efficiency. Advantageously, the straight neck used in cartridge 1230 and 1330 has a larger internal diameter of about 8.65 millimeters and a smaller outer diameter of about 10.85 millimeters. This larger internal diameter allows for faster powder filling than with a standard cartridge neck as it is wider and thus allows a greater amount of powder to be filled into the cartridge within a given time.

As shown in FIG. 13B, which shows delivery device 1300 in a pre-activated state, stopper 1333 engages male threaded portion 1314 of syringe socket 1310 in screw-threaded connection, thus ensuring correct axial alignment of cartridge 1330 with syringe socket 1310 and thus also ensuring correct alignment of fluid passage 1313 with a stopper cavity 1334 located in a proximal face of stopper 1333. Correct axial alignment of cartridge 1330 within syringe socket 1310 ensures that proximal end 1088 of needle 1080 pierces through a relatively thin central portion of stopper 1333 to reach stopper cavity 1334 during activation of delivery device 1300.

FIG. 13E shows delivery device 1300 in its activated state, with proximal end 1088 extending through fluid passage 1313 (a proximal part of which extends centrally through male threaded portion 1314), through a central, lesser-thickness part of stopper 1333 and into stopper cavity 1334, to establish fluid communication between the interior volume of the second chamber of cartridge 1330 to the outside of delivery device 1300 via needle fluid channel 1081.

Stopper 1333 is disposed in the distal end of cartridge 1330, such that the distal face of stopper 1333 is interior of, and substantially level with, an end face, annulus or rim at the distal extremity of cartridge 1330, or is at least adjacent thereto. Stopper 1333 is located substantially within the cylindrical wall of cartridge 1330 in a compressive interference fit, which provides a fluid impermeable seal therewith in a similar manner to pistons 140, 150. However, stopper 1333 is not intended to experience substantial axial movement as it is anchored to syringe socket 1310 by its screw-threaded connection with male threaded portion 1314. Thus, while stopper 1333 is formed like a piston, it does not behave as such.

Apart from delivery device 1300 being activated by connection of needle 1080 to male luer end 1312 of syringe socket 1310, delivery device 1300 may otherwise be used according to the process shown and described in relation to FIGS. 2A to 2F or 5A to 5F. Delivery devices 1000, 1200 and 1300 all use a shortened activation cap 1060.

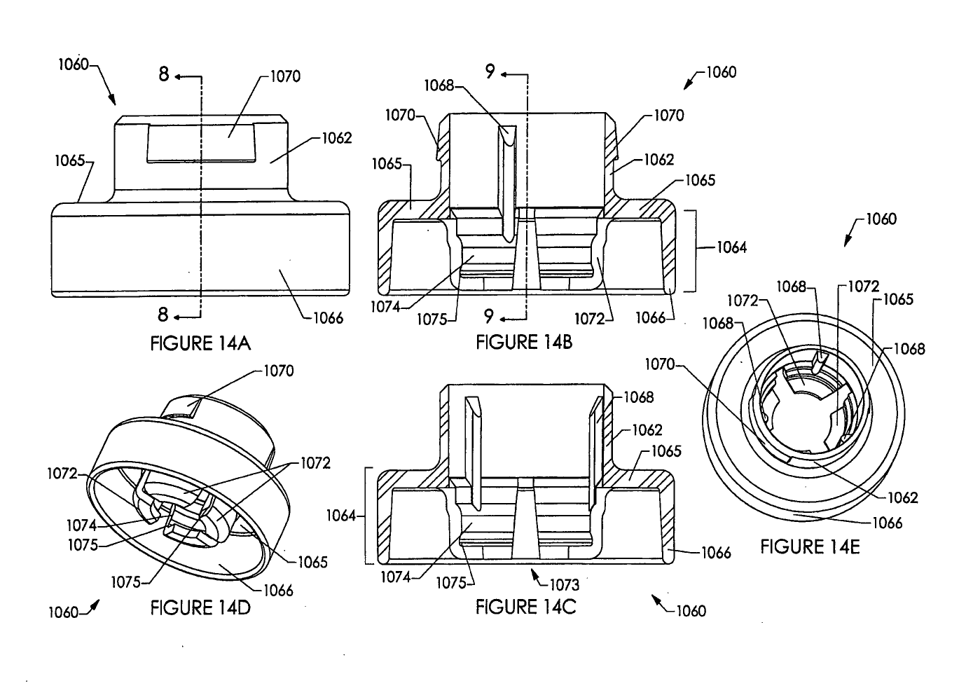

Activation cap 1060 is shown in further detail in FIGS. 14A to 14E. The activation cap 1060 shown in FIGS. 14A to 14E is substantially the same as activation cap 160 shown in FIGS. 9A to 9E, except that sleeve 1062 is shorter than sleeve 162 and it does not have a protruding rim 163 extending in a radial direction from the distal end of the sleeve. Activation cap 1060 does not have such a protruding rim because it is not necessary for activation cap 1060 to be involved in activating any of delivery devices 1000, 1200 or 1300. Further, activation cap 1060 does not have a longitudinal sleeve recess analogous to sleeve recess 169 of activation cap 160, as there is no protruding rim which requires the ability to be resiliently deflected.

Apart from the noted differences, activation cap 1060 is identical in form and function to activation cap 160. The reference numerals shown in FIGS. 14A to 14E are used to indicate corresponding features and functions as between the two activation cap embodiments and, for ease of reference, FIGS. 14A to 14E use reference numerals for such features and functions that are the same as those used in FIGS. 9A to 9E, except that they are shifted higher by the number 900. For example, base portion 1064 corresponds to base portion 164, and so on.

Referring now to FIGS. 15A to 15D, there is shown a delivery device 1500 according to yet another embodiment. Delivery device 1500 uses the same cartridge 130 as delivery device 100, but employs a modified activation cap 1560 and a modified syringe socket 1510.

Syringe socket 1510 has a proximal end 1517 identical to that of syringe socket 110, but has a modified distal end 1515. Distal end 1515 has a protractible luer 1511 slidably disposed therein in a pre-activation position of delivery device 1500. Protractible luer 1511 has a spike 1514 for penetrating a thin central portion of stopper 133 during activation of delivery device 1500. Spike 1514 has a fluid passage 1513 extending therethrough from a proximal tip of spike 1514 to a distal opening of a male luer fitting 1512 formed at the distal end of protractible luer 1511. Spike 1514 is integrally formed as part of protractible luer 1511 and is axially movable (along with protractible luer 1511) in a distal direction during activation.

Protractible luer 1511 is shown in FIG. 15A at its proximal-most position within syringe socket 1510. In this pre-activation position, proximal end flanges 1524 of protractible luer 1511 abut one or more internal circumferential ribs 1530 extending internally within wall 1516 of syringe socket 1510. Distal internal ribs 1530 project inwardly sufficiently to prevent proximal flanges 1524 from progressing proximally past distal internal ribs 1530. Thus, movement of protractible luer 1511 within syringe socket 1510 is limited in the proximal direction by contact of flanges 1524 with distal internal ribs 1530 and in the distal direction by contact of flanges 1524 with an internal end wall 1531 within distal end 1515.

When protracted distally within syringe socket 1510, protractible luer 1511 has a substantial part thereof extending distally of the distal extremity of a neck 1533 in distal end 1515. In this protracted position, proximal flanges 1524 lie against or adjacent internal end wall 1531 to limit further distal movement of the protractible luer 1511 with respect to syringe socket 1510. This is shown in FIG. 15D, which illustrates delivery device 1500 in an activated position.

Protractible luer 1511 has resilient fingers 1525 located on opposite sides of protractible luer 1511. When protractible luer 1511 is moved to its distal-most (protracted) position within syringe socket 1510, resilient fingers 1525 are resiliently deflected inwards as they pass over internal bosses 1527 within the internal wall of neck 1533 of syringe socket 1510 through which protractible luer 1511 distally protracts. Once resilient fingers 1525 pass distally beyond internal bosses 1527, resilient fingers 1525 return to a slightly outwardly biased position such that, any proximal movement of protractible luer 1511 would be prevented by the interference of a flat proximal face of resilient fingers 1525 with flat distal faces 1529 of internal bosses 1527. Thus, once protractible luer 1511 is protracted to its distal-most position, it cannot return in the proximal direction.

As protractible luer 1511 passes through neck 1533, alignment ribs 1523 on protractible luer 1511 pass between internal bosses 1527 within neck 1533 in an interlocking or interleaving fashion in order to prevent rotation of protractible luer 1511 within syringe socket 1510. Resilient fingers 1525 are located on opposite sides of protractible luer 1511 so that they extend generally proximally and slightly outwardly between adjacent pairs of alignment ribs 1523. Thus, as protractible luer 1511 protracts through neck 1533 the pair of alignment ribs 1523 adjacent each resilient finger 1525 will pass on either side of an internal boss 1527 as the respective resilient finger 1525 passes over that internal boss 1527.

Because of the cooperating structures of protractible luer 1511 and the distal end 1515 of syringe socket 1510, protractible luer 1511 is, in its distal-most position within syringe socket 1510, substantially prevented from relative axial or rotational movement with respect to syringe socket 1510.

To assist in aligning alignment ribs 1523 with internal bosses 1527 in an interleaving fashion as protractible luer 1511 protracts through neck 1533, alignment ribs 1523 are provided with angled distal ends 1526 and internal bosses 1527 are provided with angled proximal ends 1528. During such protraction, if alignment ribs 1523 and internal bosses 1527 are not oriented so as to interleave with one another, their respective angled distal ends 1526 and angled proximal ends 1528 will contact each other and their angled faces will cause relative rotational movement therebetween with further axial movement to correctly align alignment ribs 1523 and internal bosses 1527 for interleaving engagement.

Because of the protracting movement of protractible luer 1511 in a distal direction during activation of delivery device 1500, activation cap 1560 holds cartridge 130 more proximally within syringe socket 1510 than in the previous embodiments and thus requires a sleeve 1562 of greater length than sleeve 162 of activation cap 160. Aside from sleeve 1562 being of a greater length, activation cap 1560 is identical to activation cap 160, including, for example, provision of corresponding longitudinal recesses 1569, protruding rim 1563, locking sleeve protrusions 1570, apron 1566, flange 1565, grips 1572 and ase opening 1573.

Activation of delivery device 1500 is achieved in a similar manner to that described in relation to delivery device 100, except that it uses protractible luer 1511. In particular, delivery device 1500 is activated by pushing activation cap 1560 and flange 1518 of syringe socket 1510 together and thereby pushing stopper 133 on to spike 1514 and pushing protractible luer 1511 within neck 1533 to its distal-most position. As shown in FIG. 15D, in the activated position, cartridge cap 135 passes distal internal ribs 1530 as it approaches internal end wall 1531. Accordingly, distal internal ribs 1530 are sized to project radially inwardly to a degree sufficient to prevent flanges 1524 from passing proximally, but also sufficient to allow the distal end of cartridge 130, including cartridge cap 135, to pass distally during activation.

Once delivery device 1500 is placed in its activated position, a needle, such as needle 180, may be fitted on to male luer end 1512 and delivery device 1500 may be used, aside from the different activation step, in a manner substantially similar to that shown and described in relation to FIGS. 2A to 2F or FIGS. 5A to 5F.

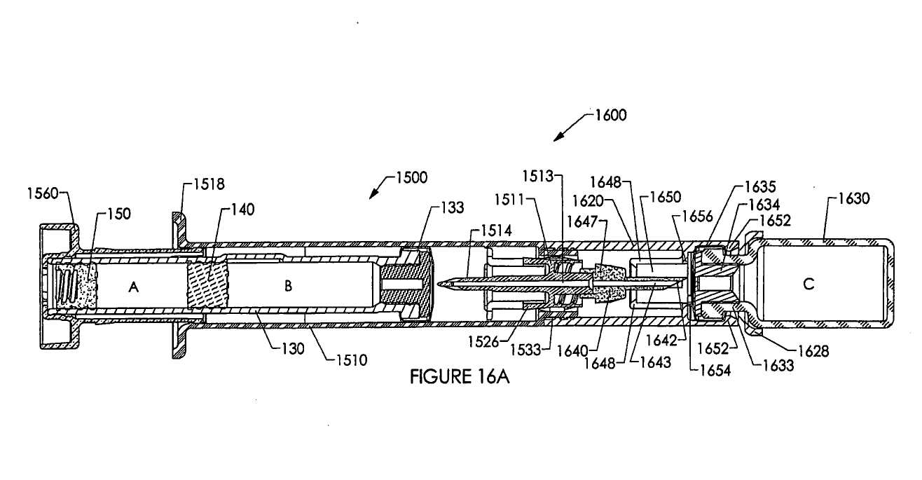

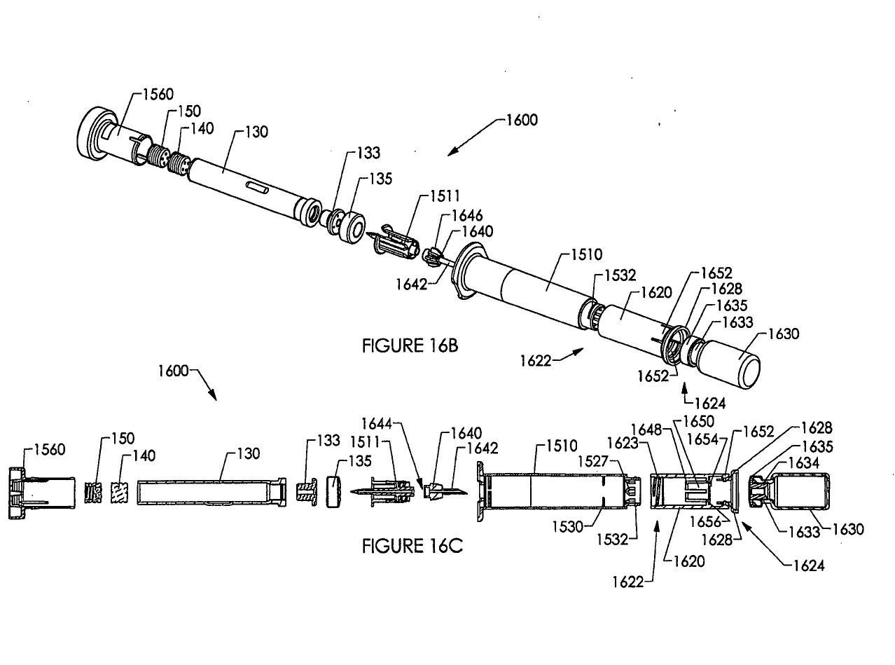

Reference is now made to FIGS. 16A to 16C, in which there is shown a mixing device 1600 according to one embodiment, as well as to FIGS. 15A to 15D. Mixing device 1600 incorporates delivery device 1500 and further includes a connector sleeve 1620 and a vial 1630. Mixing device 1600 is arranged to allow constituents A and B in the first and second chambers of cartridge 130 to be mixed with a third constituent C in vial 1630 through fluid passages interconnecting cartridge 130 with vial 1630. As indicated in the drawings, constituents A, B and C correspond generally to the first, second and third chambers, respectively.

In order to connect delivery device 1500 to vial 1630, connector sleeve 1620 has internal female thread 1623 at its proximal end 1622 for engaging lugs 1532 (or other form of male thread) on neck 1533 on the distal end of syringe socket 1510 in screw-threaded engagement. Connector sleeve 1620 further comprises an opening at its distal end 1624 for receiving a head portion 1632 and neck 1633 of vial 1630 in a latching manner, which prevents axial movement of vial 1630 relative to connector sleeve 1620.

Mixing device 1600 further comprises a fluid connector 1640 for establishing fluid communication, in an activated position of mixing device 1600, between fluid passage 1513 of protractible luer 1511 and the interior volume of vial 1630 via a fluid passage 1643 within fluid connector 1640.

FIG. 16A shows mixing device 1600 in its pre-activated position, with delivery device 1500 similarly in its pre-activated position, whereby fluid communication is not established between the respective interior volumes of cartridge 130 and vial 1630. Mixing device 1600 is activated in the same manner as activation of delivery device 1500.

When activation cap 1560 and flange 1518 of syringe socket 1510 are pushed together so that flange 1518 overlies, and is adjacent to, flange 1565 of activation cap 1560, protractible luer 1511 is forced to move distally through neck 1533. As a result, fluid connector 1640, which receives the hollow tip of male luer 1512, is pushed distally within fluid connector 1620 by protractible luer 1511 so that a spike 1642 on a distal end of fluid connector 1640 is forced to puncture a thin central portion of a stopper 1634 in the head portion 1632 of vial 1630.

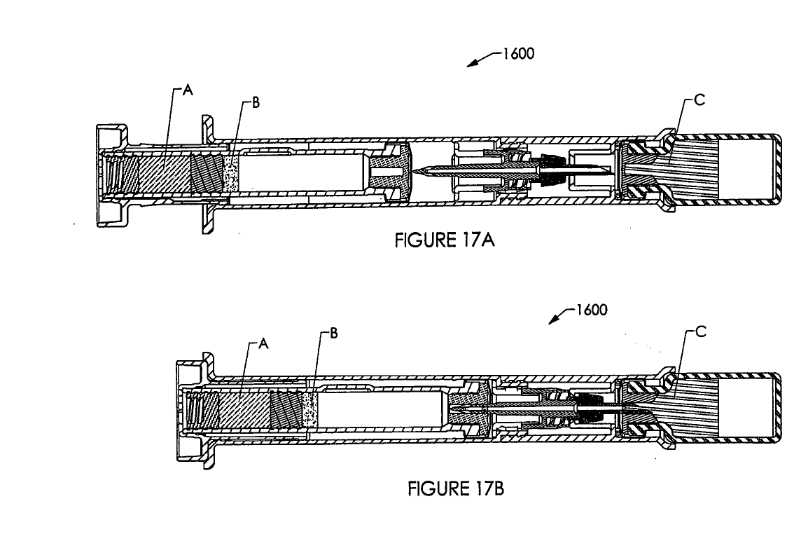

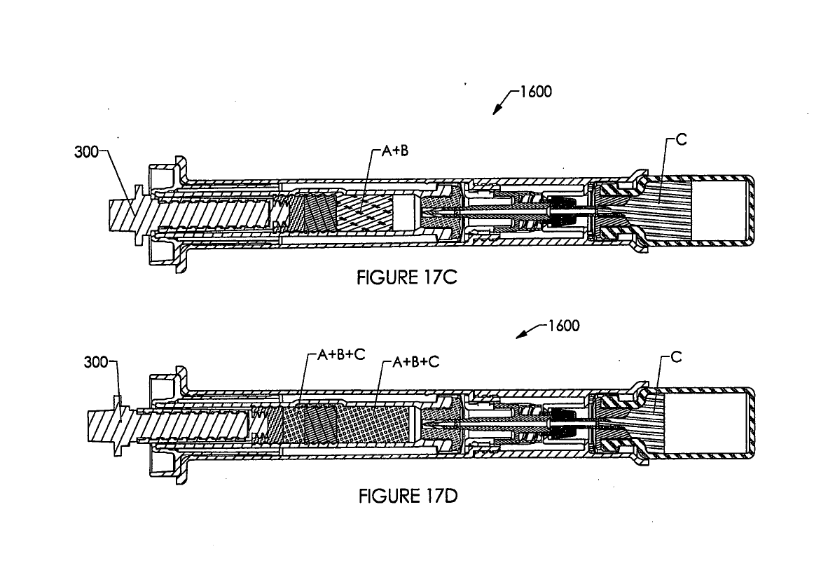

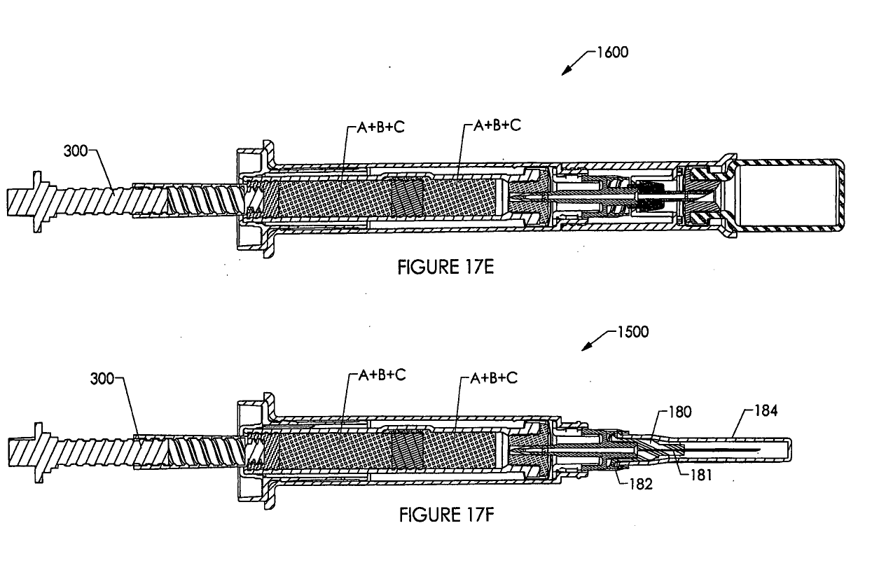

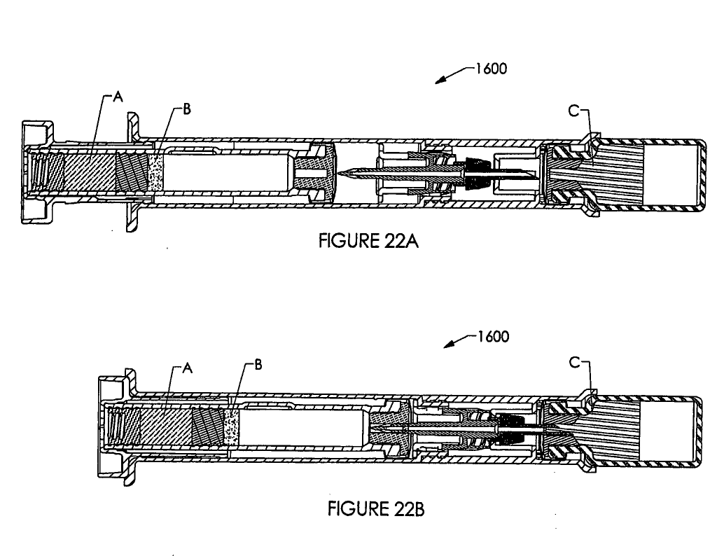

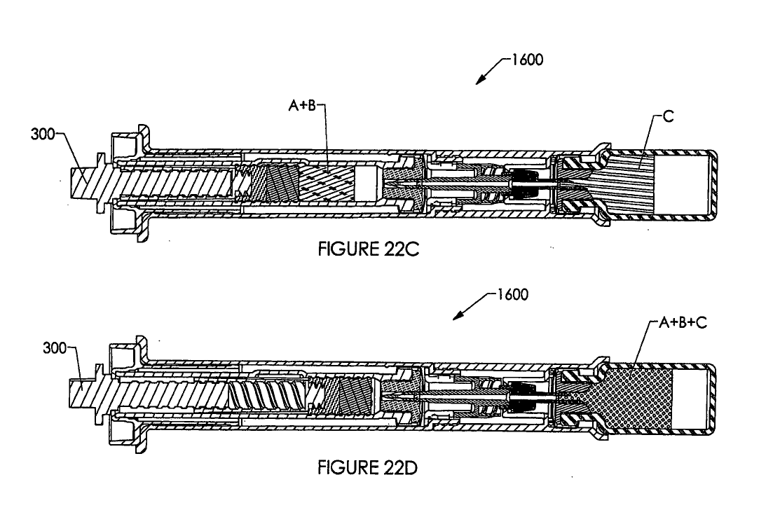

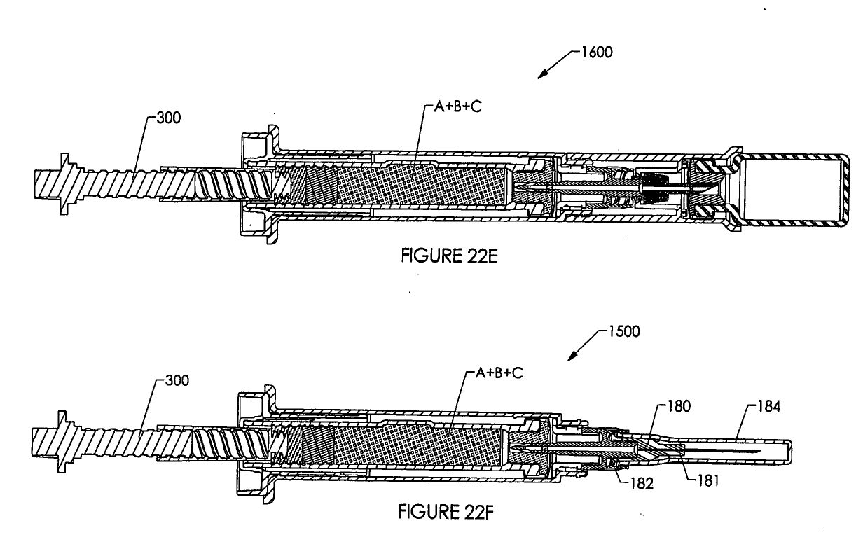

Pushing activation cap 1560 and flange 1518 together thus causes a fluid communication passage to be formed between the second chamber of cartridge 130 and the interior volume of vial 1630 via respective fluid passages 1513 and 1643 of protractible luer 1511 and fluid connector 1640. The activated position of mixing device 1600 is shown in FIG. 17B. Processes of use of mixing device 1600 are described further below, in relation to FIGS. 17A to 17F and 22A to 22F.

Generally, embodiments of the delivery and mixing devices shown and described herein are intended for single use only. Accordingly, once constituents A and B or constituents A, B and C are mixed and delivered to a patient or other subject, the delivery/mixing devices described herein are not intended to be used for any further mixing or delivery functions.

Once constituents A, B and C are mixed in mixing device 1600, connector sleeve 1620 is detached by unscrewing female thread 1623 from lugs 1532 on neck 1533 of syringe socket 1510 and a needle, such as needle 180, is connected to male luer end 1512 for subsequent delivery of the mixed constituents. Upon removal of connector sleeve 1620 from syringe socket 1510, fluid connector 1640 is retained by connector sleeve 1620 within retaining fingers 1648 of a fluid connector guide 1650. Further, the empty vial 1630 is retained by resilient latching fingers 1652 on connector sleeve 1620, which latch around the neck 1633 of vial 1630 and prevent removal of vial 1630 from connector sleeve 1620.

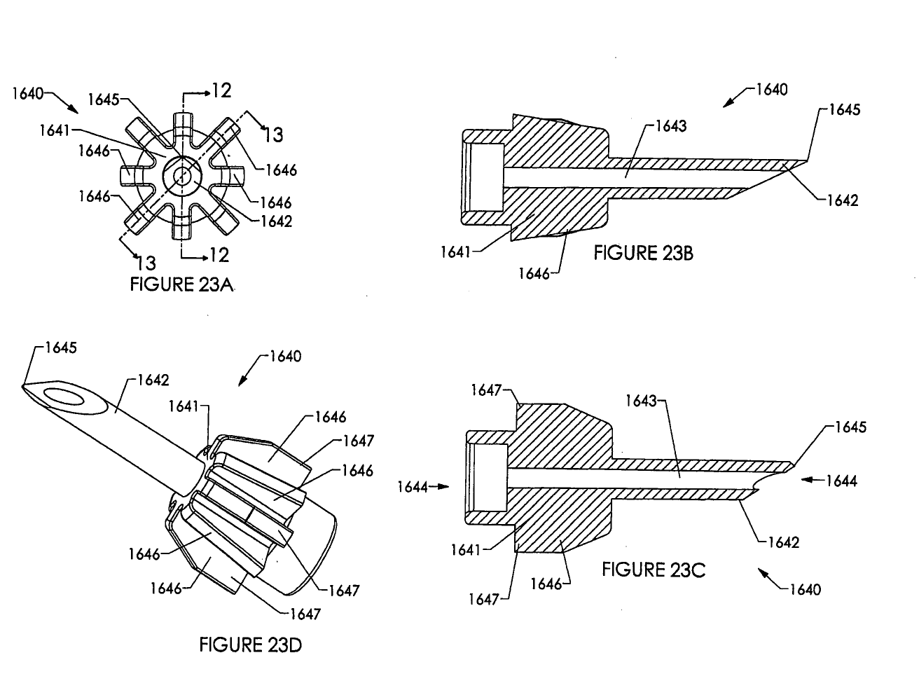

Fluid connector 1640 is shown in further detail in FIGS. 23A to 23D. Fluid connector 1640 has a proximal opening 1644 within a proximal end thereof and fluid passage 1643 extends between the proximal opening 1644 and a distal opening at a distal end of spike 1642. Spike 1642 extends distally of a main body portion 1641 of the fluid connector 1640. The proximal opening 1644 is disposed proximally of main body portion 1640 and is defined by a proximally extending cylindrical wall arranged to snugly fit around the distal extremity of the central cylindrical portion of male luer fitting 1512 though which fluid passage 1513 passes.

Spike 1642 has a tip portion shaped as if it were created from an angled slice through a tubular member. Spikes 114 and 1642 are shaped similarly at their tip portions. The tip portion of spike 1642 is shaped to substantially exclude a stopper cavity of stopper 1634 when inserted therein. To further sharpen the tip portion of spike 1642, segments are preferably cut from the distal part of the tip portion to create a sharpened tip 1645, as is visible in FIG. 23D.

Fluid connector 1640 has a plurality of radially extending wings 1646 protruding from main body portion 1641 between spike 1642 and the proximally extending cylindrical wall which defines proximal opening 1644. Wings 1646 are not all shaped the same. Preferably, eight wings 1646 are provided in an evenly spaced manner around main body portion 1641 so as to define four opposite pairs of wings, with each pair having the same radially extending shape on opposite sides of main body portion 1641. However, each circumferentially adjacent wing 1646 is different. Alternating wings 1646 have lesser and greater degrees of radial projection, as is evident in the plan view of fluid connector 1640 shown in FIG. 23A.

Wings 1646 that project radially outward to a greater degree are formed so as to have a shoulder 1647 at the proximal part of the radially projecting portion of the wing 1646. This shoulder 1647 is dimensioned to cooperate with retaining fingers 1648 to prevent proximal movement of fluid connector 1640 within connector sleeve 1620 once mixing device 1600 is placed in an activated position. This helps to ensure that mixing device 1600 can only be used once.

As an alternative configuration of fluid connector 1640, a continuous projecting shoulder may be used, instead of having discrete wings 1646. Other forms of projection from main body portion 1641 may be employed, providing some form of shoulder is provided against which retaining fingers 1648 can act to prevent fluid connector 1640 from being withdrawn proximally from connector guide 1650.

In order for connector sleeve 1620 to retain vial 1630 in its distal end, connector sleeve 1620 has a substantially cylindrical opening for receiving the head portion 1632 of the vial 1630 and resilient latching fingers 1652 for latching around the neck 1633 of vial 1630. Resilient latching fingers 1652 are resiliently moveable outward as the head portion 1632 is forced in to the distal cylindrical opening of connector sleeve 1620. Once the head portion 1632 is fully received within the opening, resilient latching fingers 1652 are biased to return to their normal position and latch around the neck 1633 of vial 1630.

With vial 1630 fully inserted in the distal end of connector sleeve 1620, a distal end flange or apron 1628 overlies an outer proximal shoulder of vial 1630 for stabilizing vial 1630 within connector sleeve 1620. As shown in FIGS. 16A to 16C, 17A to 17F and 22A to 22F, connector sleeve 1620 does not entirely enclose vial 1630, rather only enclosing the head portion 1632, neck 1633 and proximal shoulder thereof. However connector sleeve 1620 may, in an alternative embodiment, extend more distally to enclose substantially all of vial 1630.

The distal cylindrical opening of connector sleeve 1620 which receives head portion 1632 of vial 1630 is defined by the cylindrical wall of connector sleeve 1620, as well as by a flange 1656 projecting radially inwardly from the wall of connector sleeve 1620. This flange 1656 acts as a base from which connector guide 1650 and retaining fingers 1648 project proximally and from which at least two seating bosses 1654 project distally to assist in correctly seating head portion 1632 within the distal cylindrical opening of connector sleeve 1620.

Seating bosses 1654 project distally of flange 1656 adjacent the cylindrical wall of connector sleeve 1620 so as to contact vial cap 1635 toward a radial edge portion thereof and thereby prevent further proximal movement of the vial 1630 within connector sleeve 1620.

Referring now to FIGS. 17A to 17F, a process of use of mixing device 1600 is described. Mixing device 1600 is shown in FIG. 17A in its pre-activated position, in which there is no fluid communication between vial 1630 and cartridge 130. In the pre-activated position, cartridge 130 has separate constituents A and B located in respective first and second chambers of cartridge 130 and vial 1630 holds a third constituent C. Vial 1630 thus acts as a third chamber of mixing device 1600.

As shown in FIG. 17A, the first, second and third chambers of mixing device 1600 are longitudinally disposed in sequence moving from the proximal end of mixing device 1600 to its distal end. Fluid connection may be established between the first and second chambers within cartridge 130 via bypass channel 139 and fluid communication may be established between the second chamber and the third chamber via the fluid passage 1513 of protractible luer 1511 and the fluid passage 1643 of fluid connector 1640, which are both substantially linearly disposed in an axial and longitudinal direction within the generally cylindrical walls of syringe socket 1510 and connector sleeve 1620.

With the mixing device 1600, constituents A and B may be easily and conveniently mixed. To do this, constituents A and B are first mixed within cartridge 130 and the mixed constituents A and B are then mixed with the third constituent C either within vial 1630 (as described in relation to FIGS. 22A to 22F), or within cartridge 130.

FIG. 17B shows mixing device 1600 in an activated state, in which flange 1518 and activation cap 1560 have been pushed together so as to force cartridge 130 to move distally within syringe socket 1510 and thereby impale stopper 133 on spike 1514, puncturing stopper 133 and fluidly connecting fluid passage 1513 with the interior volume of cartridge 130.

Pushing cartridge 130 distally within syringe socket 1510 also causes fluid connector 1640 to move distally within connector sleeve 1620 so as to puncture stopper 1634 of vial 1630 with spike 1642 and thus create a fluid connection between fluid passage 1643 and the interior volume of vial 1630. As fluid connector 1640 is engaged around the distal tip of male luer 1512, the fluid passage 1643 of fluid connector 1640 is fluidly connected to the fluid passage 1513 of protractible luer 1511. Through the fluid connections thus formed, fluid can be communicated between cartridge 130 and vial 1630.

In FIG. 17C, mixing device 1600 is shown with a plunger 300 having been actuated in a first stroke so as to mix constituents A and B by forcing constituent A to flow through bypass channel 139 from the first chamber into the second chamber. By forcing constituent A through bypass channel 139, the first chamber is effectively collapsed and the second chamber now holds a mix of constituents A and B. This mixing is caused by the fluid dynamics of constituent A as it flows in to the second chamber and the tendency of constituent B, which is usually in a powder form, to dissolve or become suspended in the fluid of constituent A. In mixing device 1600 as shown in FIG. 17C, constituent C has not yet been mixed with constituents A and B.

As shown in FIG. 17D, upon withdrawal of plunger 300 from cartridge 130, constituent C is caused to flow through fluid passage 1640 and fluid passage 1513 into the second chamber of cartridge 130, thereby mixing constituents C with mixed constituents A and B. As central piston 140 is not acted upon directly by plunger 300, it remains in a position aligned with bypass channel 139 as plunger 300 is withdrawn, while actuating piston 150 is moved proximally by virtue of its attachment to plunger 300. This proximal movement of actuating piston 150 causes a vacuum between the pistons into which the contents of the second chamber flows, including mixed constituents A, B and C.

FIG. 17E shows mixing device 1600 with plunger 300 substantially withdrawn from cartridge 130 and in its extended position. Because of the full withdrawal of plunger 300 from cartridge 130, the full contents of vial 1630 is aspirated into the second chamber of cartridge 130 and, via bypass channel 139, into the recreated first chamber. The three constituents having been mixed and being contained in cartridge 130, connector sleeve 1620 can be removed, thus removing fluid connector 1640 from the end of male luer 1512.

With connector sleeve 1620 removed, a needle, such as needle 180, can be connected to male luer 1512 for delivering the mixed constituents by actuation of plunger 300, as shown in FIG. 17F. With connector sleeve 1620, fluid connector 1640 and vial 1630 removed by unscrewing connector sleeve 1620 from syringe socket 1510, mixing device 1600 is transformed into delivery device 1500. Delivery device 1500 can then be used to deliver the mixed constituents via needle 180.

FIGS. 17A to 17F depict one possible process of use of mixing device 1600. Another embodiment is shown and described in relation to FIGS. 22A to 22F.

Referring now to FIGS. 18A to 18D, the passage of fluid from the first chamber of cartridge 130 into the second chamber via bypass channel 139 is illustrated. A generic plunger P is shown in FIGS. 18A to 18D for illustration purposes only. While a straight, inextensible plunger may be employed with embodiments of the invention, it is preferred to use an extensible plunger, such as plunger 300, as shown and described in relation to FIGS. 3A to 4C or plunger 600, as shown and described in relation to FIGS. 6A to 7C. Alternatively, an inextensible plunger, such as is shown and described in relation to FIGS. 25A and 25B may be employed.

In FIG. 18A, cartridge 130 is shown with central piston 140 being located proximally of bypass channel 139 and thus fluidly separating constituents A and B from each other. In FIG. 18B, plunger P is actuated so as to push actuating piston 150 in a distal direction. As the fluid in the first chamber (including constituent A and probably some air) does not have any means of escape from the first chamber and is compressible only to a limited extent, distal movement of actuating piston 150 with cartridge 130 also causes central piston 140 to be moved distally because of fluid pressure within the first chamber.

Once central piston 140 is moved distally enough so that its proximal face is positioned distally of the proximal-most extent of bypass channel 139, the fluid in the first chamber, under pressure from actuating piston 150, flows into bypass channel 139, around the outer surface 142 of piston 140 and into the second chamber to mix with constituent B. For this to occur, bypass channel 139 is longer than the distance between the proximal and distal faces of central piston 140.

FIG. 18C shows actuating piston 150 being moved sufficiently distally to collapse the first chamber and force all of constituent A into the second chamber to mix with constituent B. Once the bypass channel 139 is open to flow from the first chamber, central piston 140 is no longer pushed distally by fluid pressure in the first chamber. Once actuating piston 150 actually contacts central piston 140, it may again be moved distally. As is shown in FIG. 18D, further distal actuation of actuating piston 150 acts directly on central piston 140 to move it distally and displace the contents of the second chamber, being mixed constituents A and B, through fluid channel 1513 of protractible luer 1511 in the distal direction shown by arrow 1800.

Referring now to FIGS. 19A and 19B, there is shown a delivery device 1900 according to a further embodiment. Delivery device 1900 is identical to delivery device 1500, except that cartridge 130 uses modified central and actuating pistons 1940, 1950.

Modified pistons 1940, 1950 are adapted to interlock with each other when actuating piston 1950 is forced distally against central piston 1940. This means that, once the first chamber of cartridge 130 is collapsed during mixing of constituent A with constituent B, and actuating piston 1950 is pressed against central piston 1940 to interlock therewith, the two pistons can be moved axially together, either in a proximal direction or a distal direction, without separating. Accordingly, if delivery device 1900 is used instead of delivery device 1500 in mixing device 1600, the contents of the vial can be aspirated into the cartridge 130 without separation of the two pistons during withdrawal of the plunger in the proximal direction.

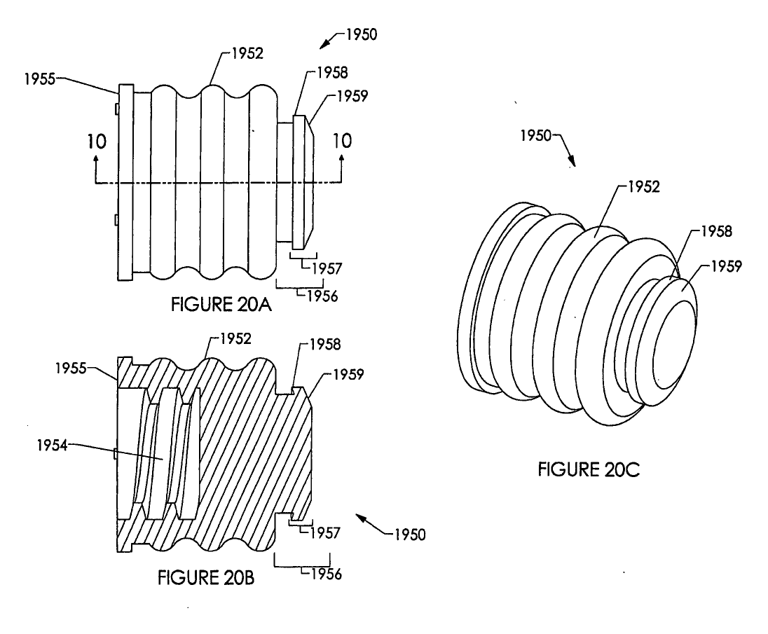

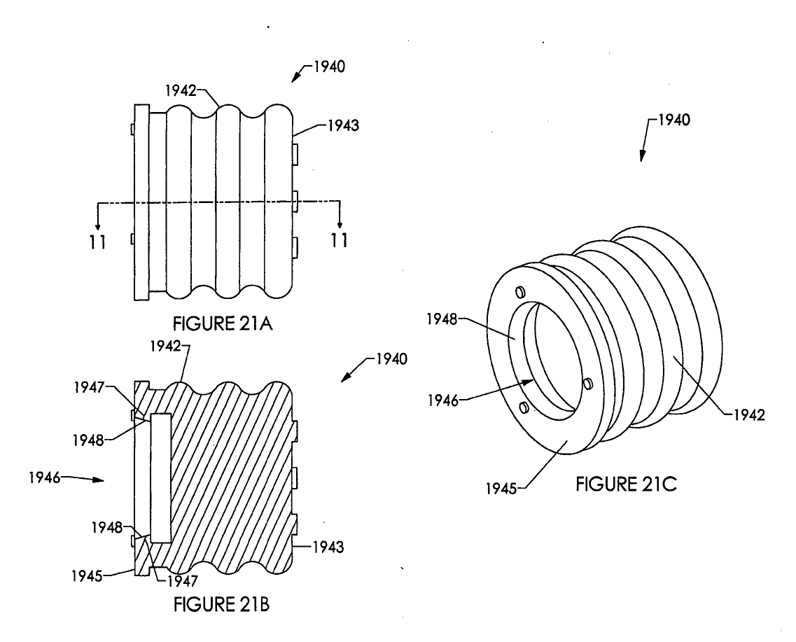

FIG. 19B shows delivery device 1900 with plunger 600 connected to actuating piston 1950, which is in turn connected to central piston 1940, following delivery of the contents of cartridge 130. Referring also to FIGS. 20A to 20C and FIGS. 21A to 21C, actuating piston 1950 and central piston 1940 are shown in further detail. Actuating piston 1950 is similar to actuating piston 150, except that it has a male projection 1956 for interlocking with a corresponding recess 1946 in the proximal face 1945 of central piston 1940.

Actuating piston 1950 has a plunger socket 1954 extending into the piston from proximal surface 1955 and has an outer surface 1952 for forming a fluid impermeable seal with the inner surface of cartridge wall 131. Projection 1956 extends distally from a distal surface of actuating piston 1950 and has a head portion 1957 on the distal end of projection 1956. Head portion 1957 comprises a radial flange 1958, which is slightly proximally directed and extends radially of a neck of projection 1956. Head portion 1957 also comprises a sloped head surface 1959 of a substantially frustoconical form for assisting to guide projection 1956 centrally within recess 1946 of central piston 1940.

Central piston 1940 is substantially identical to central piston 140, except that it has the recess 1946 extending within its body from proximal surface 1945. Central piston 1940 otherwise has an outer surface 1942 similar to that of central piston 140 and a distal surface 1943 similar to that of central piston 140. Recess 1946 is defined by an inwardly angled flange 1947 at the mouth thereof and a generally cylindrical wall extending distally of angled flange 1947 and slightly radially outward thereof.

Recess 1946 is shaped to receive projection 1956 in an interlocking fashion when the distal face of actuating piston 1950 is pressed towards the proximal face of central piston 1940 so that the head portion 1957 is received within the cylindrical walls of recess 1946. As projection 1956 starts to enter recess 1946, sloped head surface 1959 of projection 1956 contacts a sloped interior flange wall 1948, which assists to centrally register and inwardly guide projection 1956 within recess 1946. Once projection 1956 is fully received within recess 1946, inward angled flange 1947 lies proximally of radial flange 1958 and prevents proximal movement thereof, thereby effectively locking or latching central piston 1940 to actuating piston 1950.

While actuating piston 1950 and central piston 1940 are shown and described as employing a mating projection and recess, respectively, it will be appreciated that other forms of latching or interlocking pistons may be employed. Further, pistons 140, 150, 1940 and 1950 are generally shown in the drawings as having small bosses on the distal and proximal surfaces thereof. These small bosses are used to separate the pistons from each other and ensure that they do not stick together during initial manufacture and handling. It should be appreciated that such bosses are not essential to the operation of the invention but are shown here for completeness.

While delivery device and mixing device embodiments have been shown and described herein as using a single bypass channel 139 arranged as a longitudinal groove extending radially outward of the rest of the interior and exterior surfaces of the cartridge wall, an alternative bypass means may be formed in an alternative fashion in any or all of the described embodiments. In particular, multiple bypass channels may be employed, a shallower channel may be used, which does not result in perturbation of the exterior surface of the cartridge, or one or more bypass channels may be formed by deforming central piston 140 or 1940 as it passes a radially inwardly directed projection or deformation of the cartridge wall. Alternatively, the bypass may be formed in or through the central piston.

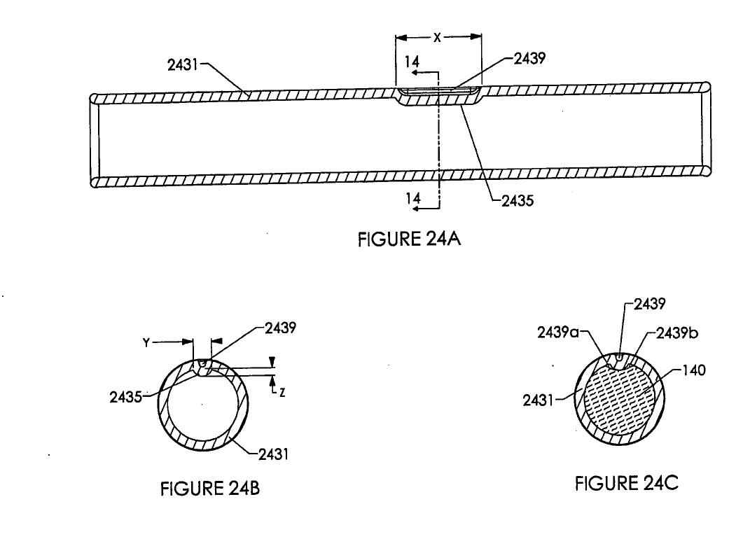

In one particular alternative embodiment, bypass channels may be formed by inwardly deforming the wall of the cartridge, in an inverse manner to the outward deformation associated with bypass channel 139, as shown in FIGS. 24A to 24C. This inward deformation of the wall causes the central piston to inwardly compress as it passes the deformation and thus come away from the interior surface of the cartridge and break its fluid impermeable seal therewith, whereupon fluid may flow around the central piston, along two small channels formed adjacent the inward deformation between the inwardly deformed central piston and the cartridge wall adjacent the deformation.

This inward deformation of the cartridge wall is formed during manufacture of the cartridge tube while the glass of the cartridge is relatively soft and may advantageously be formed by depressing the cartridge wall from the outside, which is easier than forming an internal ridge by accretion, moulding or other form of deposit. Further advantageously, bypass channels which result in widening of the outer surface of the cartridge at any point, such as bypass channel 139, may create handling difficulties. This is because much of the manufacturing machinery for handling cartridges is arranged to handle cartridges of a certain constant external diameter.

Referring now to FIGS. 22A to 22F, there is shown a further method of use of mixing device 1600. FIGS. 22A to 22C are identical to FIGS. 17A to 17C and thus the description of FIGS. 17A to 17C applies to FIGS. 22A to 22C. However, in FIG. 22D, instead of aspirating constituent C into the cartridge 130, mixed constituents A and B are injected into vial 1630 to mix with constituent C, after which, as shown in FIG. 22E, mixed constituents A, B and C are aspirated together into cartridge 130 as plunger 300 is withdrawn proximally.

Following aspiration of mixed constituents A, B and C into cartridge 130, connector sleeve 1620, fluid connector 1640 and vial 1630 are removed and replaced by a needle 180, as shown in FIG. 22F, in preparation for injection of the mixed constituents through needle 180.

Aspiration of mixed constituents A, B and C into cartridge 130, as shown in FIG. 22E, so that the mixed constituents are contained within the expanded second chamber of the cartridge 130, relies on formation of a vacuum between pistons 140 and 150 so that they can be moved proximally together. If formation of such a vacuum proves difficult or unreliable using pistons 140 and 150, these pistons may be replaced by pistons 1940 and 1950, respectively to ensure that the pistons interlock with each other and can be moved proximally together. Accordingly, in another embodiment of the method of use shown in FIGS. 22A to 22F, pistons 140 and 150 are replaced with pistons 1940 and 1950, respectively.

Referring now to FIGS. 24A to 24C, there is shown a cartridge tube 2430 for use in a cartridge according to another embodiment. Cartridge tube 2430 uses an internal bypass structure instead of an external bypass. Tube 2430 is identical to the cartridge tube used in cartridges 1230 and 1330, except that an inwardly directed recess 2439 is formed in the outer surface of wall 2431 of tube 2430. This is in contrast to the outwardly directed recess described and shown in relation to other embodiments.

Inward recess 2439 is formed by deforming the wall 2431 of tube 2430 so as to create an inward deformation 2435 of the wall 2431, which projects inwardly so as to deform the central piston 140 as the piston passes recess 2439. Because of the shape of the deformation 2435 of the inner surface of wall 2431, piston 140 is inwardly compressed around where it contacts the inward deformation 2435 as it is forced to pass thereby. This compression of piston 140 forces the outer surface of central piston 140 away from its sealing contact with the inner surface of the wall 2431 and creates small bypass channels 2439a, 2439b on either side of the inward deformation 2435 of the tube wall 2431. Thus, when central piston 140 is longitudinally aligned between the ends of recess 2439, central piston 140 is in a bypass position and fluid may be transmitted around central piston 140 via bypass channels 2439a, 2439b. The longitudinal length of the cartridge tube 2430 across which the recess 2439 and inward deformation 2435 extend may be considered to be a bypass portion of the wall 2431.

Cartridge tube 2430 is used for mixing constituents in the manner shown and described in relation to FIGS. 18A to 18D, despite using an internal bypass instead of an external bypass.

Recess 2439 may be formed by, for example, pressing radially inwardly with an appropriately shaped tool head while the glass of tube 2431 is softened by heat. This heat softening is preferably localized to that part of wall 2431 which is to be inwardly deformed. In order to prevent the wall 2431 from caving under application of the tool head, an internal support may be used to support the inside surface of the wall on either side of where the inward deformation 2435 is to be formed, thereby allowing for a relatively sharp transition between inward deformation 2435 and the surrounding inner surface of the wall of tube 2431. This sharp transition must be sufficiently angled so that central piston 140 cannot maintain its fluid impermeable seal with the inner surface of the cartridge wall around inward deformation 2435.

The longitudinal length X of recess 2439 is preferably about 13.3 millimeters, plus or minus 0.75 millimeters. The distal end of recess 2439 is preferably located proximally of the distal extremity of tube 2430 by about 26.5 millimeters. The length X and position of recess 2439 may be varied somewhat, according to particular requirements.

The width of inward deformation 2435 is indicated by Y in FIG. 24B. The width Y extends between the transitions on either side of inward deformation 2435 from the cylindrical inner surface of wall 2431 to inward deformation 2435. Width Y is preferably about 2.25 millimeters, plus or minus 0.2 millimeters.

The depth of inward deformation 2435 as it projects inwardly of the cylindrical inner surface of wall 2431 is indicated by Z in FIG. 24B. The depth Z is measured as the maximum radial distance between the crest of inward deformation 2435 and the nominal radius of the inner surface, which is the point between the crest and the recess 2439 where the inner surface of the tube wall 2431 would have been, but for the inward deformation 2435. Depth Z is preferably between about 0.8 millimeters and 1.00 millimeters, although some small variation outside this range may be workable.

If the depth Z is too great, the central piston 140 may not be able to be pushed past inward deformation 2435 or the inward deformation 2435 may interfere with actuation of the plunger as it passes the inward deformation 2435. If depth Z is too shallow, central piston 140 may not be adequately inwardly compressed to separate its outer surface from the inner surface of the wall 2431 or the bypass channels 2439a, 2439b may be so small as to be inadequate for fluid flow of fluids above a certain viscosity.

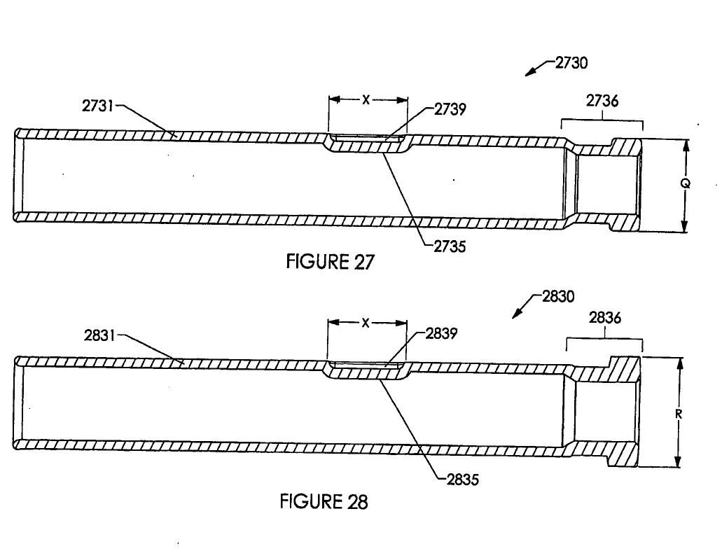

While bypass recess 2439 and inward deformation 2435 are shown in FIGS. 24A to 24C in relation to a straight cylindrical cartridge tube 2430 (i.e. with a straight neck portion 2436), such as is used in delivery devices 1200 and 1300, the same internal bypass formation may be applied to more standard cartridge forms (i.e. with detented and flanged neck portions), such as those shown and described in relation to delivery devices 100, 1000, 1100, 1500 and 1900 and in mixing device 1600. An example of the internal bypass applied to a cartridge tube having a standard 13 mm (outside diameter) neck is shown in FIG. 28. A modified version of the standard neck, having a reduced outside diameter of about 1 mm, is shown in FIG. 27.

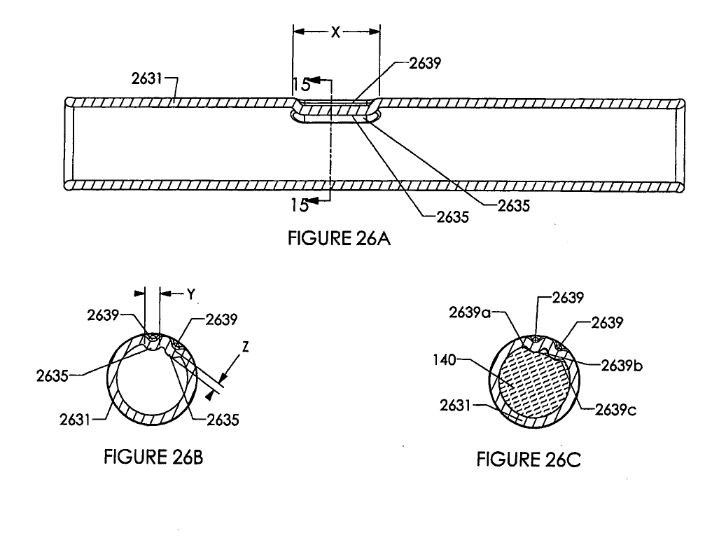

An alternative form of the internal bypass is shown in FIGS. 26A to 26C, in which more than one inward deformation 2635 is formed in the wall 2631 of cartridge tube 2630, located closely adjacent to each other. If more than one such internal bypass is used, care should be taken to avoid requiring excessive compression of central piston 140 or interfering with passage of the plunger. Adjacent inward deformations 2635 advantageously define a groove or notch therebetween, which acts as a further bypass channel. In such a case, inward deformations 2635 may not need to have a generally semi-circular cross section, as is shown in FIGS. 24B and 24C and may not need to have the same depth Z as described above.

Adjacent inward deformations 2635 advantageously may facilitate the formation of three separate bypass channels 2639a, 2639b, and 2639c, as shown in FIG. 26C, when central piston 140 is compressed in the bypass position. Bypass channel 2639b is formed between the adjacent inward deformations 2635, while bypass channels 2639a and 2639c are formed on opposite sides of the two inward deformations 2635. Depending on the depth Z of inward deformations 2635, bypass channels 2639a and 2639c may be too small to allow appreciable fluid flow therethrough. However, even a smaller depth Z of inward deformations 2635 than that of inward deformation 2435 will compress central piston 140 sufficiently to allow the central bypass channel 2639b to be formed. Thus the depth Z of inward deformations 2635 may be less than that of inward deformation 2436 while still facilitating the formation of one or more suitable bypass channels.

Aside from the formation of two inward deformations instead of one, cartridge tube 2630 is otherwise identical to tube 2430. As with cartridge tube 2430, the straight neck portion 2636 may be substituted with a standard or modified neck portion 2836 or 2736, as shown in FIGS. 28 and 27, respectively. The length X and width Y of each of recesses 2639 is preferably about the same as that of recess 2439. Recesses 2639 and their corresponding inward deformations 2635 are preferably formed simultaneously using a two-headed tool and pressing radially inwardly on wall 2631 when the glass is softened by heat. Alternatively, recesses 2639 may be formed in succession. In either case, appropriate supports may be provided on the inner surface of wall 2631 adjacent recess 2639 during formation thereof in order to prevent the wall 2631 from caving in.

In a further embodiment, the formation of the inward deformations may be performed by first forming an external bypass (such as is shown in cartridge 130), for example by vacuum from the outside of the tube, and then pushing the tube wall on both sides of the external bypass inwards, resulting in the two adjacent inward deformations defining a bypass channel through what was initially formed as the external bypass, as shown in FIGS. 26A, 26B and 26C. These steps are performed while the glass is heated and therefore more readily deformable.

As shown in FIGS. 26A to 26C, adjacent inward deformations 2635 are angularly separated by about 35 degrees from crest to crest. In order to achieve adjacent location of inward deformations 2635, their crests are preferably angularly separated by between about 30 degrees and 40 degrees. While adjacent inward deformations are preferred, in an alternative form of the internal bypass, inward deformations 2635 may be spaced from each other around the circumference of the inner surface of tube 2631, for example at about 90 degrees or 180 degrees from each other.

In an alternative embodiment (not shown) more than two inward deformations 2635 may be provided, projecting inwardly from the inner surface of tube 2631. For example, three or four such inward deformations may be provided, with some or all of them being adjacent. Advantageously, bypass channels may be readily formed with three or four such inward deformations. However, the compressibility of central piston 140 should be such that it can pass through the bypass portion with relative ease, despite the increased friction associated with three or four inward deformations. Further, it may be desirable to employ a plunger having a relatively small diameter in order to avoid the shaft of the plunger interfering with the inward deformations as it passes through the bypass portion.

In a further alternative (not shown), inward deformations 2635 may be formed so as to have a cross-sectional shape (as seen in FIGS. 26B and 26C) other than a simple rounded inward projection of wall 2631. For example, inward deformations 2635 may be shallower or more angularly formed by using a different tool head or internal support structure during formation of the inward deformations 2635. So long as the inward deformations 2635 define a bypass channel with respect to the central piston 140 as it passes through the bypass portion of tube 2630, various cross-sectional shapes or profiles may be used to form inward deformations 2635. Accordingly, in order to form such alternative shapes of inward deformation, the direction and force of application of the pressing tool or tools may not be directed strictly radially inwardly but may have a small tangential component.

Referring now to FIGS. 27 and 28, there are shown alternative cartridge tubes 2730 and 2830, respectively. Cartridge tubes 2730 and 2830 are identical to cartridge tube 2430, except that they have different neck portions 2736, 2836. Cartridge tube 2830 has a neck portion 2836 of a standard 13 mm outside diameter form similar to cartridge tube 130. The outside diameter of cartridge tube 2830 is given by R in FIG. 28. R is preferably about 13 mm, but this may vary slightly, depending on manufacturing tolerances or the particular use for which it is intended. Neck portion 2736 of cartridge tube 2730 has an outside diameter Q of about 11 mm. Neck portion 2736 is slightly detented in the distal direction before widening into a flanged portion at the distal end having outside diameter Q. Cartridge tube 2730 has the advantage of a small neck diameter, while still providing a flange around which an end cap (such as end cap 3135 shown in FIG. 31) may be secured to hold the stopper in place.

As with cartridge tube 2430, cartridge tubes 2730, 2830 have a substantially cylindrical tubular wall 2731, 2831, with a bypass recess 2739, 2839 formed therein and a corresponding inward deformation 2735, 2835 extending inwardly of the inner surface of the wall 2731, 2831. In further alternative embodiments, cartridge tubes 2730, 2830 may be formed with multiple inward deformations 2735, 2835 in a similar manner to cartridge tube 2630, as described above.

Referring now to FIGS. 25A and 25B, there is shown a plunger 2500 according an alternative embodiment. Plunger 2500 is inextensible and has a length dimensioned so as to extend as far into the cartridge as is necessary, while a proximal end flange 2514 remains proximal of the proximal opening of the cartridge. Plunger 2500 has an elongate shaft 2515 extending between proximal flange 2514 and a piston engaging portion 2524 on the distal end of the shaft 2515. A plurality of ridges 2523 are formed around shaft 2515 along a distal portion of plunger 2500. These ridges extend circumferentially, either continuously or discontinuously, around the outside of shaft 2515 and are formed in succession longitudinally along shaft 2515.

Ridges 2523, as seen in side cross-section in FIG. 25A, have a saw-tooth configuration such that, when plunger 2500 is inserted into the cartridge during actuation of the delivery or mixing device while mixing constituents A and B, ridges 2523 interfere with base grips 172 in succession in a ratcheting manner. This interference mitigates against an overly vigorous stroke of the plunger 2500 during mixing of the constituents and, by virtue of the saw-tooth configuration of ridges 2523, prevents withdrawal of plunger 2500 in the proximal direction. This is because ridges 2523 are each sloped outwardly in a proximal direction with a sharp inward transition to define a shoulder, which would abut a distal face of base grips 172 to limit proximal movement of plunger 2500. Thus, plunger 2500 is suitable for single use devices.

In an alternative embodiment of plunger 2500, rounded corrugations may be used in place of sharp ridges 2523, so as to allow proximal withdrawal of the plunger from the cartridge, while still interfering with base grips 172 and thus retarding movement of the plunger.

The ridges 2523 or corrugations are formed along a distal portion of shaft 2515 of a length corresponding to the distance of movement required for actuating piston 150 to travel between its initial position and the position at which the first chamber is collapsed completely (shown in FIG. 18C). The remainder of shaft 2515 between the ridges or corrugations and end flange 2514 is substantially cylindrical.

As an alternative, or in addition to ridges or corrugations, inextensible plunger 2500 may have a taper extending the length of the portion on which the ridges or corrugations are disposed. Such a taper is substantially similar to the taper employed by the sleeves of plungers 300 and 600 and extends outwardly in the proximal direction.

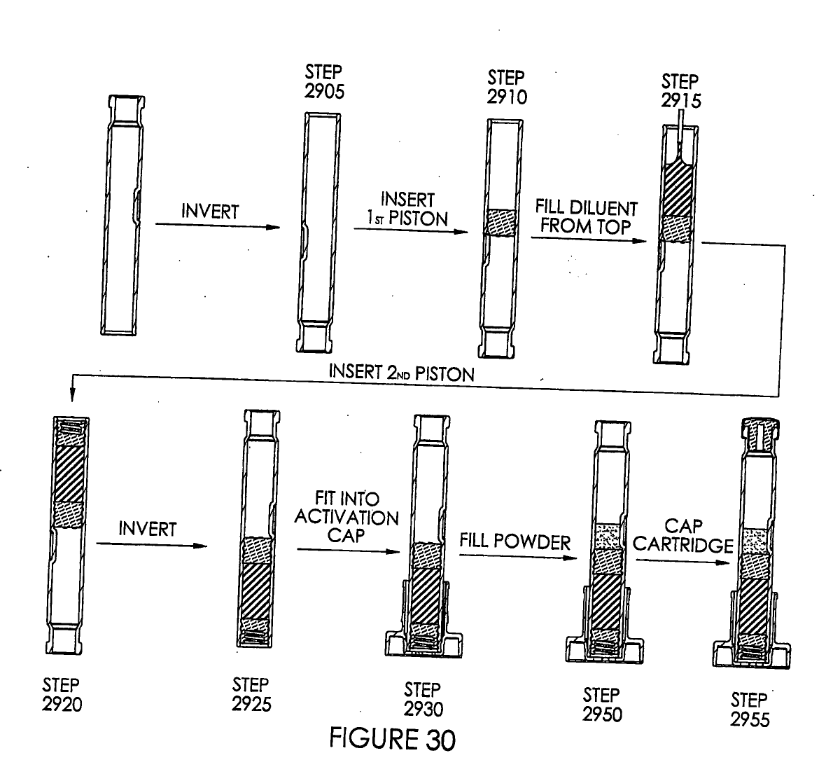

Referring now to FIGS. 29 and 30, a cartridge filling method 2900 is described. Cartridge filling method 2900 is illustrated using one exemplary cartridge tube corresponding to cartridge tube 2730, although it may be substituted for any other cartridge tube shown and/or described in this application. Cartridge filling method 2900 is an improvement of a cartridge filling method shown and described in U.S. patent application Ser. No. 10/951,039, filed Sep. 25, 2004, entitled “System for Filling and Assembling Pharmaceutical Delivery Devices”, the entire contents of which is hereby incorporated by reference.

While the steps of method 2900 are shown as part of a process flow diagram in FIG. 29, some of the steps of method 2900 are also shown in FIG. 30 in a corresponding pictorial representation. Method 2900 begins at step 2905 with the provision of an inverted cartridge tube 2730. At step 2910, the first (central) piston 140 is inserted into cartridge tube 2730 so that it is positioned between the bypass portion and the first (bottom) end of cartridge tube 2730. Because cartridge tube 2730 is shown inverted in FIG. 30, the bottom end is shown as being located upwardly of the second (top) end.

In step 2915, the first constituent is filled through the bottom end into the cartridge tube 2730 in an open first chamber defined by the central piston 140 and the wall 2731. The first constituent is preferably a diluent such as sterilized water. The water may also be demineralized.

Once the diluent has been filled into cartridge tube 2730 through its bottom opening, the second (actuating) piston 150 is inserted into cartridge tube 2730 through the bottom opening to close the first chamber and seal the diluent within the first chamber. Actuating piston 150 is positioned so as to remain relatively close to the bottom opening of cartridge tube 2730, while being wholly received therein, so as to permit ready engagement of a plunger with actuating piston 150. This insertion of the actuating piston 150 into cartridge tube 2730 is performed as step 2920. After such insertion, the cartridge tube 2730 is inverted, at step 2925, so as to place the neck portion of the cartridge tube 2730 towards the top and locate the bottom end, including actuating piston 150, towards the bottom. Following steps 2920 and 2925, the cartridge tube 2730 is partially filled. In its partially filled state, the cartridge tube 2730 may be stored, sold as such, used to form a partially filled cartridge assembly or all of these.

At step 2930, the bottom end of the cartridge tube 2730 is fitted into an activation cap, such as activation cap 160. In this context, activation cap 160 acts as a support cap for stabilizing the cartridge for transportation in an upright orientation. This is facilitated by the larger footprint conferred by activation cap 160 than would be conferred by that of the cartridge tube 2730 on its own. Depending on the activation method to be used when mixing the constituents, activation cap 1060 or 1560 may be substituted for activation cap 160 in the filled or partially filled cartridge assembly.

In one alternative form of method 2900, steps 2925 and 2930 may be performed in an opposite order, so that the activation cap 160 is fitted onto the bottom end of the cartridge tube 2730, after which the assembly is inverted for subsequent transport.

The partially filled cartridge assembly may be sterilized at step 2935, if desired. Alternatively, this sterilization step may be omitted. At step 2940, the partially filled cartridge assembly (comprising the partially filled cartridge and the activation cap) is transported, preferably in a sterile environment, to a powder filling facility located separately from the diluent filling facility. At the powder filling facility, the partially filled cartridge assembly is sterilized, at step 2945, for example by autoclaving or other suitable sterilization techniques. Following sterilization step 2945, the second constituent, which may be a drug in powder form, is filled into cartridge tube 2730, between central piston 140 and neck portion 2736 through the open top end.

Following powder filling, cartridge tube 2730 is capped, as step 2955, by insertion of a stopper into the top opening and placement of a cap over the stopper and at least part of the neck portion 2736, thereby sealing the top end of the cartridge, enclosing the second constituent in a second chamber of the cartridge and forming a filled cartridge assembly. Steps 2950 and 2955 are performed in a sterile environment within the powder filling facility.

FIG. 31 shows a filled cartridge assembly 3100, as formed by cartridge filling method 2900. Filled cartridge assembly 3100 comprises the cartridge tube 2730 received within activation cap 160 (which may also be considered as a base support cap because of its support function during transportation of the assembly). Cartridge tube 2730 contains a first constituent A sealed between central piston 140 and actuating piston 150 and a second constituent B sealed between central piston 140 and a stopper 3133 sealing the distal end (also called the top or second end) of cartridge tube 2730.

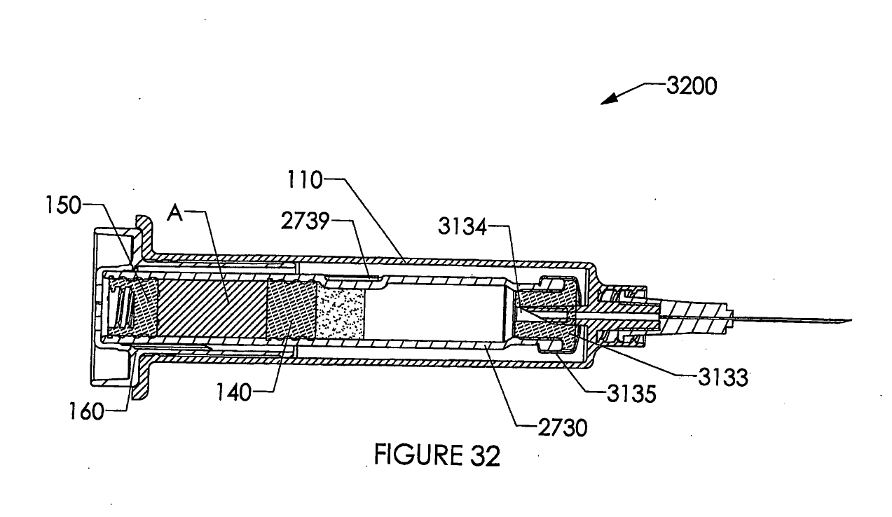

Filled cartridge assembly 3100 may be inserted into a syringe socket, such as syringe socket 110, 3310 or other syringe socket embodiments as shown and described in this application. Thus, following assembly of the filled cartridge assembly 3100, it may be inserted into a syringe socket (in a pre-activated position), activated according to a method previously described and used to mix and deliver constituents A and B in response to actuation by a plunger. FIG. 32 shows the cartridge assembly 3100 within a delivery device 3200. Delivery device 3200 is shown in its activated position and is substantially the same as delivery device 100 (as shown in FIG. 1E) except for the use of the filled cartridge assembly 3100.

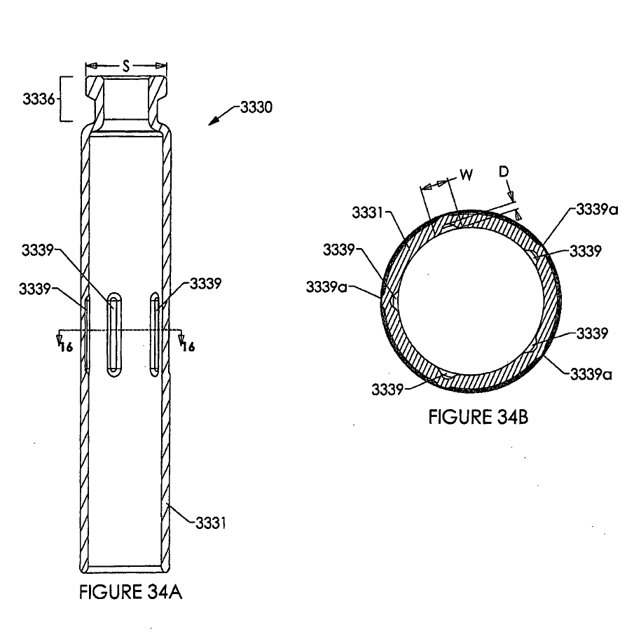

In alternative embodiments, filled cartridge assembly 3100 and delivery device 3200 employ cartridge tubes having alternative bypass configurations, such as a single external bypass channel (as shown in FIG. 1B, for example) and multiple bypass channels formed so as not to create a substantial external protrusion of the cartridge wall (as shown in FIGS. 34A and 34B, for example).

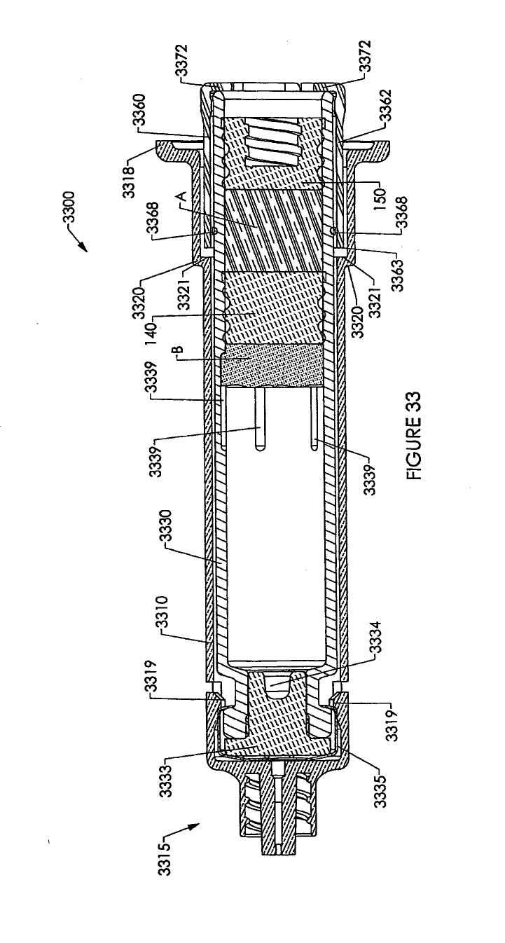

Referring now to FIGS. 33, 34A, 34B, 35A to 35C and 36A to 36D, there is shown a delivery device 3300, and some of its components, according to another embodiment. Delivery device 3300 is used in a manner similar to that described in relation to delivery device 100 or 1000, for example, although delivery device 3300 uses slightly modified components. Delivery device 3300 has a syringe socket 3310 for receiving a cartridge 3330 having constituents A and B sealed in respective first and second chambers within cartridge 3330. Central piston 140 separates constituents A and B and divides the interior of cartridge 3330 into the first and second chambers. Actuating piston 150 is positioned within the open proximal end of cartridge 3330 and a stopper 3333 is positioned in a neck 3336 of an open distal end of cartridge 3330.

Delivery device 3300 also includes a modified activation cap 3360 attached to the proximal end of cartridge 3330 and at least partially received within a proximal open end of syringe socket 3310. Activation cap 3360 has a longitudinally extending sleeve to overlie at least a part of the outer wall of cartridge 3330 and gripping fingers 3372 which resiliently snap around and grip a bead at the open proximal end of cartridge 3330. In these respects, activation cap 3360 is similar to activation cap 160 and 1060, except that it does not have a radial flange or apron extending away from sleeve 3362. Another difference is that activation cap 3360 does not have any latching projections on sleeve 3362 for engaging the inside wall of the syringe socket. Thus, activation cap 3360 is received within syringe socket 3310 in a clearance fit whereby sleeve 3362 is partly received within an enlarged base portion of syringe socket 3310 defined by a circumferential step 3320.

Step 3320 defines a shoulder 3321 on an inside surface of syringe socket 3310 for abutting a distal end face 3363 of activation cap 3360 to prevent the activation cap 3360 (and thus the cartridge 3330) from progressing too far distally within syringe socket 3310. Activation cap 3360 has a bead 3368 or other form of ridge, formed continuously or in an interrupted manner circumferentially around the inside of sleeve 3362 towards a distal end thereof. Bead 3368 helps to register and position the outer wall of cartridge 3330 within activation cap 3360 in a manner similar to that of ribs 168 or 1068.

Syringe socket 3310 is generally similar to syringe socket 1010 except for the circumferentially enlarged base portion in cylindrical wall 3316, defined by step 3320. Further, cartridge 3330 is retained within syringe socket 3310 by means of an opposed pair of resiliently deformable latching protrusions 3319 projecting slightly inwardly towards a distal end 3315 of syringe socket 3310. Latching protrusions 3319 are formed in outer cylindrical wall 3316 of syringe socket 3310 and are positioned to latch around a head and neck portion 3336 of cartridge 3330 when cartridge 3330 is fully inserted into syringe socket 3310.

In order for cartridge 3330 to become secured within syringe socket 3310, the head and neck of cartridge 3330 are pushed within syringe socket 3310 towards distal end 3315. When a cap 3335 on the distal end of cartridge 3330 contacts latching protrusions 3319 and is forced in the direction of distal end 3315, this causes latching protrusions 3319 to deflect slightly outwardly and allow progress of the head and neck portion 3336 of cartridge 3330 toward distal end 3315. In its most distally progressed position within syringe socket 3310, cartridge 3330 has its end cap 3335 positioned up against an inner distal end wall (transverse to wall 3316) of syringe socket 3310 and latching projections 3319 have returned substantially to the rest states to which they are resiliently biased and in which they substantially latch around the head and neck of cartridge 3330, including cap 3335. Because of the latching function provided by latching projections 3319 on the syringe socket 3310, activation cap 3360 is not required to provide any latching function with respect to the inner walls of syringe socket 3310 to retain cartridge 3330 within syringe socket 3310.

Stopper 3333, as illustrated in FIG. 33, has a cavity 3334 in a proximal end thereof and has a relatively thick central portion through with a needle, such as needle 1080, may be forced proximally so that it protrudes into cavity 3334 for connecting an internal volume of cartridge 3330 with an external volume. Insertion of the needle through the fluid channel in distal end 3315 and into stopper 3333 forms part of the activation step prior to mixing constituents A and B. A circular aperture (not shown) is formed in the material of cap 3335 for allowing the needle to be pushed into stopper 3333. The thickness of the rubber portion of stopper 3333 through which the needle must be inserted to reach cavity 3334 may be less than that shown, in order to more easily facilitate manual insertion of the needle through distal end 3315.

Referring in particular to FIGS. 34A and 34B, cartridge 3330 is shown in further detail, although without stopper 3333 and cap 3335 at its distal end and without pistons 140 or 150. In the illustrated embodiment of cartridge 3330, a plurality of recesses 3339 are formed in a spaced configuration circumferentially around an internal surface of cartridge tube wall 3331. Recesses 3339 have a width W, corresponding to the circumferential extent of each recess. The width W of each recess is preferably between about 1.5 mm to 0.7 mm. Each recess 3339 also has a depth D, measured as the radial distance between a nominal circumference of the inner surface of wall 3331 and the apex of the recess inside wall 3331. The depth D of each recess 3339 is preferably between about 0.5 mm to 0.8 mm, and most preferably about 0.65 mm.

Recesses 3339 may be formed within wall 3331 so as to result in a corresponding raised portion 3339a on the outer surface of wall 3331 at the location of each recess 3339. One method of formation of recesses 3339 involves use of a tool to press outwardly on wall 3331 from inside cartridge 3330. Recesses 3339 are preferably formed to have a suitable depth to allow relatively viscous fluid flow, while minimizing the degree of outward radial projection of projections 3339a. Preferably, the thickness of wall 3331 is such that recesses 3339 can be formed (by plastic deformation of the inner surface of wall 3331) with depths of up to 0.8 mm without external projections 3339a projecting radially by more than about 0.3 mm. In order to counteract the possible handling difficulties that may be encountered by external projections of even as small as 0.3 mm, wall 3331 may be compressed slightly inwardly along the bypass portion (i.e. the longitudinal extent of recesses 3339) so as to reduce the radius of the nominal outer surface of wall 3331 by a few tenths of a millimeter.

Although FIG. 34B shows five recesses 3339 formed in wall 3331, the number of recesses 3339 may be varied, for example from 2 to 6. It is preferred that, however many recesses 3339 are formed in wall 3331, they are equally spaced from each other. This is because equidistant spacing of recesses 3339 is more likely to allow even flow of the fluid constituent A into and around the circumferential channels between the ribs of central piston 140 when central piston 140 is in the bypass position. If these circumferential channels are reliably completely filled with the fluid of constituent A, the initial volume of constituent A can be provided in an amount to compensate for the entrapment and loss of the fluid in the circumferential channels, thereby providing a more accurate and consistent volume of fluid for mixture with constituent B.

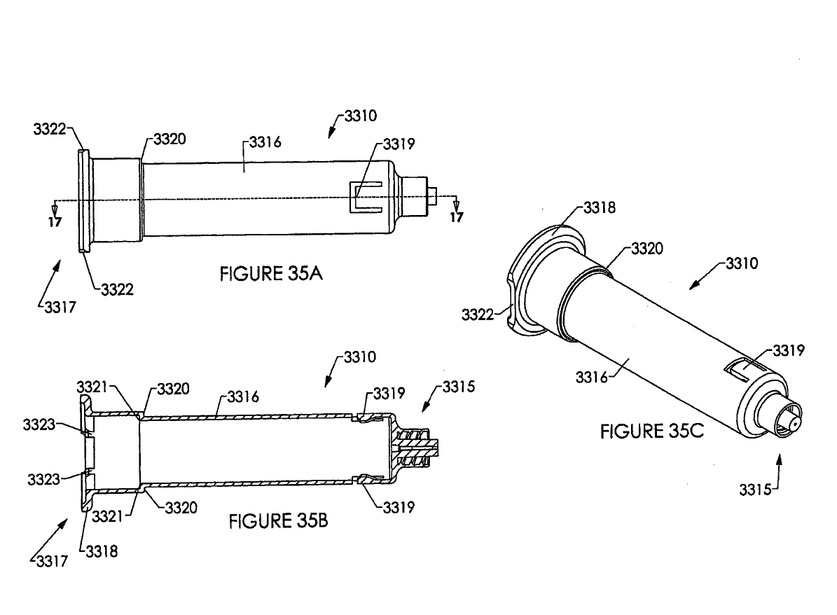

Referring in particular to FIGS. 35A to 35C, syringe socket 3310 is shown in further detail. Syringe socket 3310 has been described to some degree above in relation to FIG. 33. Similar to the syringe socket embodiment shown in FIGS. 8A to 8C, syringe socket 3310 has cap alignment bosses 3323 to assist correct axial alignment of activation cap 3360 within syringe socket 3310. These cap alignment bosses 3323 are provided around the inside of open proximal end 3317. Activation cap 3310 also has a proximal flange 3318 extending radially outwardly around proximal end 3317. Flange 3318 has flattened portions 3322 on diametrically opposite sides thereof.

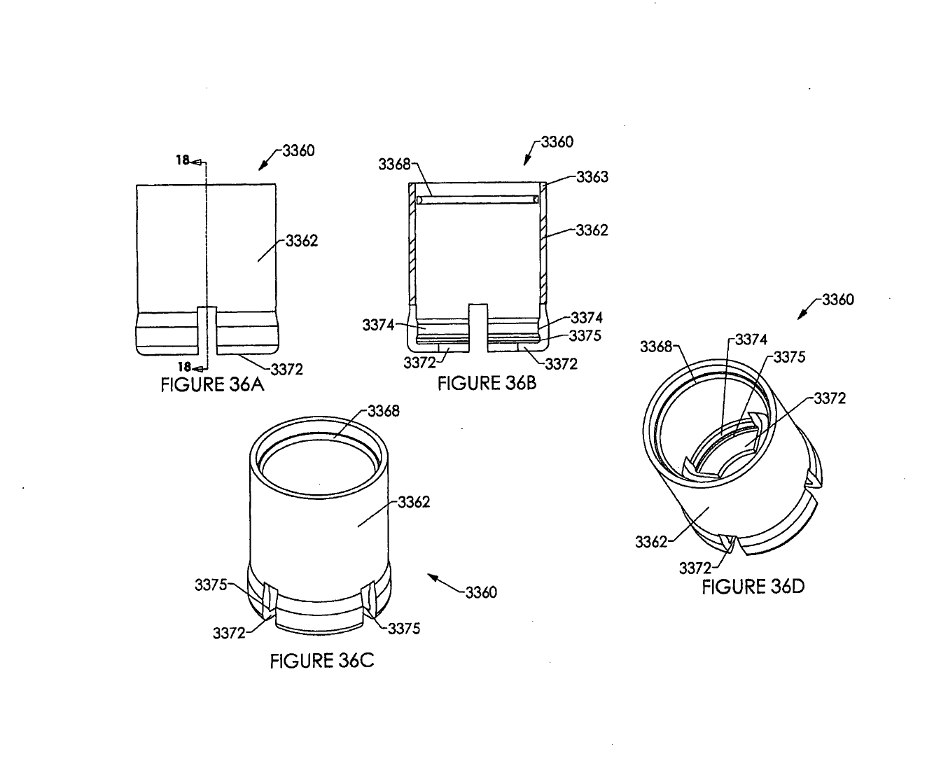

Referring in particular to FIGS. 36A to 36D, activation cap 3360 is described in further detail. Activation cap 3360 has a similar function to activation caps 160 and 1060 in so far as it overlies and grips the proximal end of cartridge 3330. The gripping of cartridge 3330 by activation cap 3360 is performed by finger grips 3372. Finger grips 3372 have a slightly inwardly projecting bead 3374 adjacent a slight radially outwardly extending recess 3375. Bead 3374 and recess 3375 are shaped for snap fitting engagement of an end bead on the proximal end of wall 3331 of cartridge 3330. As described in relation to activation cap 160 and 1060, fingers 3372 are resiliently deflectable and extend radially inwardly around the open proximal end of cartridge 3330 so as to interfere with a plunger (according to certain embodiments as described herein) as it progresses distally within cartridge 3330.

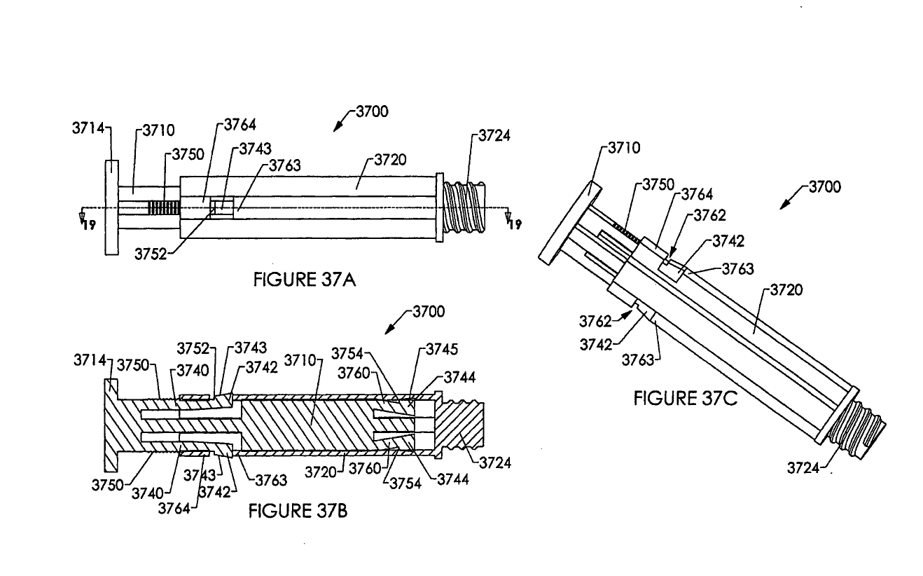

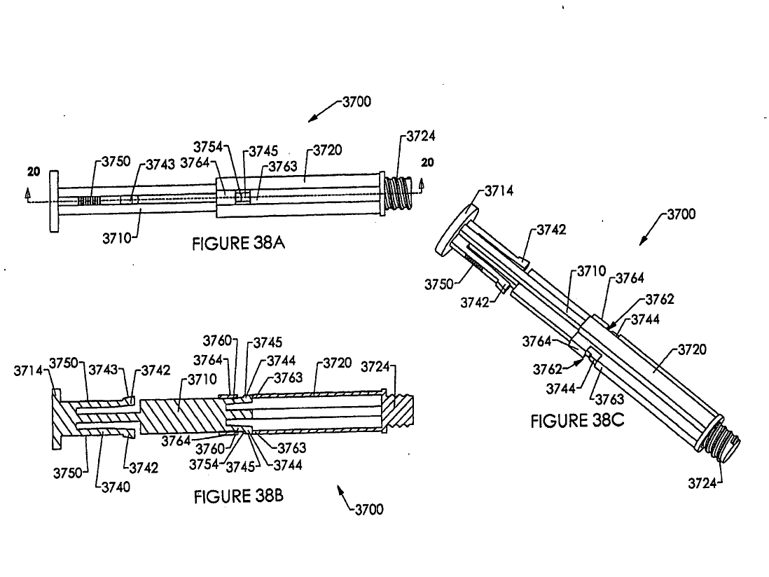

Referring now to FIGS. 37A to 37C, there is shown an extensible plunger 3700 in a retracted position. Referring also to FIGS. 38A to 38C, the extensible plunger 3700 is shown in an extended position. Plunger 3700 is arranged to extend and retract axially by means of a slidable connection between two elongate members, a shaft 3710 and a sleeve 3720. In the retracted position, most of shaft 3710 is received within sleeve 3720. In the extended position, sleeve 3720 extends substantially away from shaft 3710, although a distal end of shaft 3710 remains engaged by a proximal end of sleeve 3720. Plunger 3700 may be used in conjunction with delivery device 100 or other delivery device embodiments described herein.

A proximal head portion of shaft 3710 has an end flange 3714 for assisting with manual manipulation of plunger 3700. Flange 3714 has a proximal face and a distal face. Shaft 3710 extends distally from the distal face and has a transverse cross-section that is substantially smaller than the transverse cross-section of flange 3714.

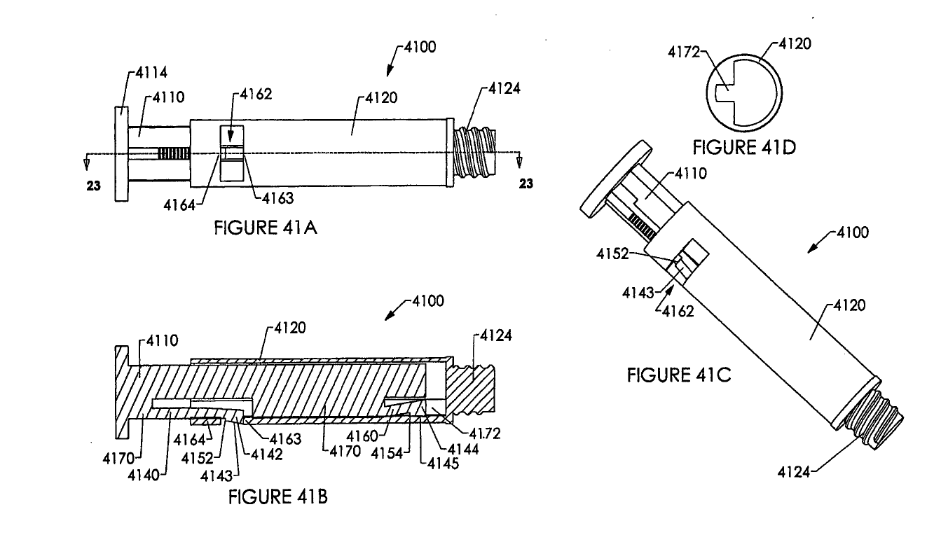

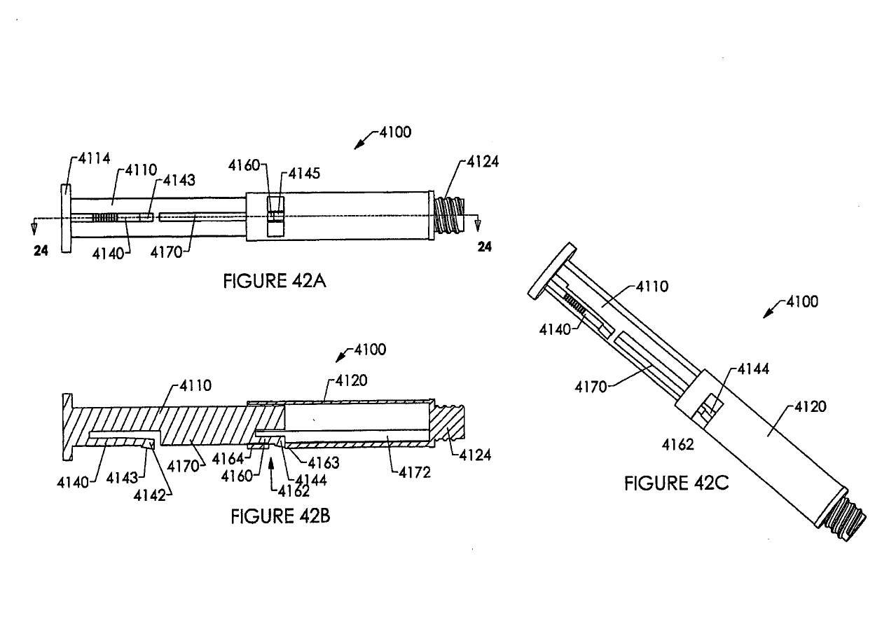

As shown in FIG. 37B, shaft 3710 is slidably received within a correspondingly shaped cavity of sleeve 3720. The shapes of shaft 3710 and the cavity are formed to prevent rotation of shaft 3710 relative to sleeve 3720. In one embodiment, the respective transverse cross-sections of shaft 3710 and the cavity of sleeve 3720 are approximately matching cruciform shapes. Alternatively, shaft 3710 may have a single rectangular key on a substantially circular cross-section, as shown in FIGS. 41A to 42C. Various other shaft and sleeve shapes may be employed, but preferably they have shapes that tend to prevent relative rotation of shaft 3710 with respect to sleeve 3720.

Sleeve 3720 also has a male threaded end 3724 at its distal extremity for screw threaded engagement with actuating piston 150 within plunger socket 154. Sleeve 3720 is substantially hollow proximally of male threaded end 3724, for slidably receiving shaft 3710.

In operation, while plunger 3700 is retained in the retracted position, flange 3714 is pressed distally on its proximal face in a first stroke, thereby placing central piston 140 in a bypass position and allowing fluid flow of constituent A from the first chamber into the second chamber to mix with constituent B. When both constituents have mixed under the action of the first plunger stroke, flange 3714 is pulled proximally away from sleeve 3720 by the distal face to extend plunger 3700, where it is retained in the extended position. In a second stroke, flange 3714 is pressed distally on its proximal face, thereby effecting delivery of the mixed constituents A and B to an external volume, such as a vial or a patient body.

To retain shaft 3710 in the retracted position, there is a pair of resiliently deflectable proximal fingers 3740 extending longitudinally along a proximal portion of shaft 3710. To retain shaft 3710 in the extended position, there is a pair of resiliently deflectable distal fingers 3760 extending longitudinally along a distal portion of shaft 3710. Fingers 3740, 3760 are integrally formed with shaft 3710 such that there are cavities in shaft 3710 that undercut each of the fingers 3740, 3760 to allow inward deflection of fingers 3740, 3760 from their undeflected rest states.

As shown in FIG. 37B, shaft 3710 is held in the retracted position by proximal projecting portions 3742 of proximal fingers 3740 that protrude into cavities 3762 within sleeve 3720. Each proximal projecting portion 3742 has a proximal tapered surface 3743 on the outward face thereof. At a proximal edge of each projecting portion 3743 is a lip 3752 that abuts a proximal edge 3764 of sleeve 3720 when shaft 3710 is pulled proximally relative to shaft 3720 to prevent inadvertent extension. A distal edge 3763 of sleeve 3720 abuts a distal face of projecting portion 3742 to prevent distal movement of shaft 3710 relative to sleeve 3720. Located between distal edge 3763 and proximal edge 3764, cavities 3762 may be sized slightly larger than projecting portions 3742 so that projecting portions 3742 may fully protrude into cavities 3762.

As shown in FIG. 38B, shaft 3710 is held in the extended position by distal projecting portions 3744 of distal fingers 3760 that protrude into cavities 3762. Distal projecting portions 3744 include a lip 3754 and a distal tapered surface 3745 and are generally similar in construction to distal projecting portions 3742.

In the retracted position, proximal fingers 3740 are received within cavities 3762 and distal fingers 3760 are retained within the interior walls of sleeve 3720. In the retracted position, distal fingers 3760 are deflected inwards by compression of distal projecting portions 3744 against the interior distal walls of sleeve 3720. In some embodiments, proximal edge 3764 of sleeve 3720 may slightly compress proximal fingers 3740 on their outer surfaces midway between a finger activation portion 3750 and proximal projecting portion 3742. This slight inward deflection improves the ability of proximal fingers 3740 to expand resiliently into cavities 3762 for retaining the position of shaft 3710 relative to sleeve 3720. In some other embodiments, proximal fingers 3740 may be in their rest state while retained within cavities 3762.

In the extended position, proximal fingers 3740 are positioned externally from sleeve 3720 in an undeflected rest state and distal fingers 3760 are received within cavities 3762. In the extended position, distal fingers 3760 function similarly to proximal fingers 3740 when in the retracted position. However, distal fingers 3760 are substantially shorter than proximal fingers 3740 and, accordingly, inward deflection of distal fingers 3760 by a user requires a greater application of force as compared to proximal fingers 3740, due to the greater amount of relative deflection required to bend distal fingers 3760. The greater resistance of distal fingers 3760 to being pressed inward in the extended position restricts a users ability to inwardly deflect distal fingers 3760 and return plunger 3700 to the retracted position. Thus, plunger 3700 is suited for a single actuation from the retracted position to the extended position as part of a single use mixing and delivery device.

If relative longitudinal movement between shaft 3710 and sleeve 3720 is desired, as with extension of plunger 3700, a user may inwardly depress finger activation portions 3750 located on proximal fingers 3740. Once proximal fingers 3740 have been deflected such that lips 3752 are positioned inwardly of proximal edge 3764, the user may pull shaft 3710 away from sleeve 3720, without lips 3752 abutting proximal edge 3764.

As shown in FIGS. 37B and 37C, proximal projecting portion 3742 has a tapered surface 3743 sloping inward toward the proximal end thereof to facilitate easier extension of plunger 3700. After slightly depressing proximal finger 3740 and pulling shaft 3710 away from sleeve 3720, tapered surface 3743 contacts proximal edge 3764 to progressively inwardly deflect proximal finger 3740 further as shaft 3710 is pulled away from sleeve 3720. Tapered surface 3743 permits a user to apply a small initial deflection before deflecting proximal projecting portion 3742 to the extents caused by contact with the interior walls of sleeve 3720. As shown in FIGS. 37A to 38C, finger activation portions 3750 include surface perturbations formed on proximal finger 3740 to assist a user in gripping finger activation portions 3750 during extension of plunger 3700.

Once all proximal projecting portions 3742 are deflected sufficiently inward, shaft 3710 may be pulled proximally by flange 3714 to extend plunger 3700. When distal fingers 3760 reach cavities 3762, they are biased outward by their own shape memory from their inwardly deflected positions. As shown in FIG. 38B, distal projecting portions 3740 protrude into cavities 3762 to retain plunger 3700 in the extended position. Similar to proximal projecting portions 3742, distal projecting portions 3744 retain plunger 3700 in the extended position by the abutment of the lips 3754 against proximal edge 3764 and the abutment of opposing faces of distal projecting portions against distal edge 3763.

In alternative embodiments, greater or fewer deflectable fingers may be used to retain shaft 3710 within sleeve 3720. For example, FIGS. 41A to 42D and 42A to 42D illustrate a plunger 4100 with one proximal deflectable finger and one distal deflectable finger. In other alternatives, different cooperating structures may be used, such as springs, latches and other mechanisms known in the art to secure a device between two alternative positions.

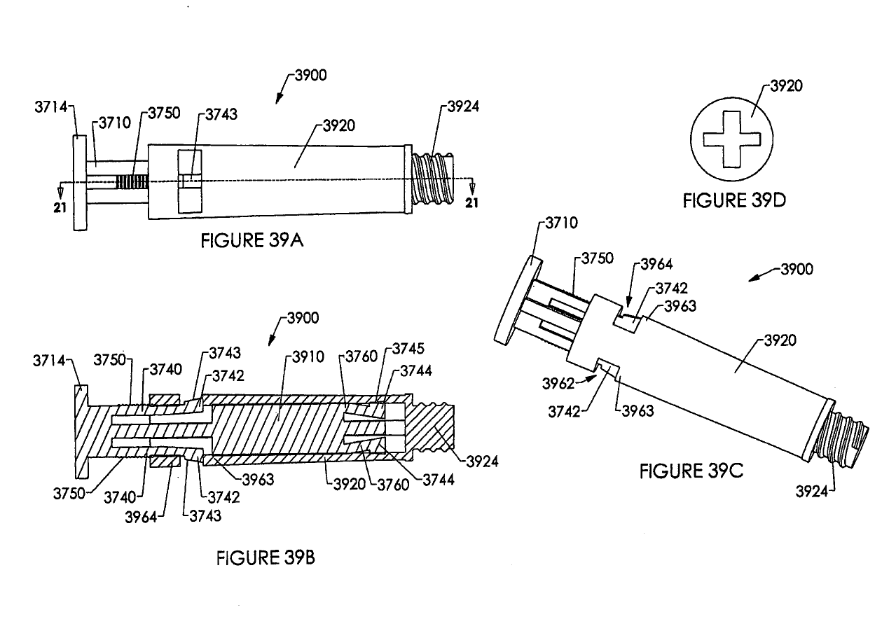

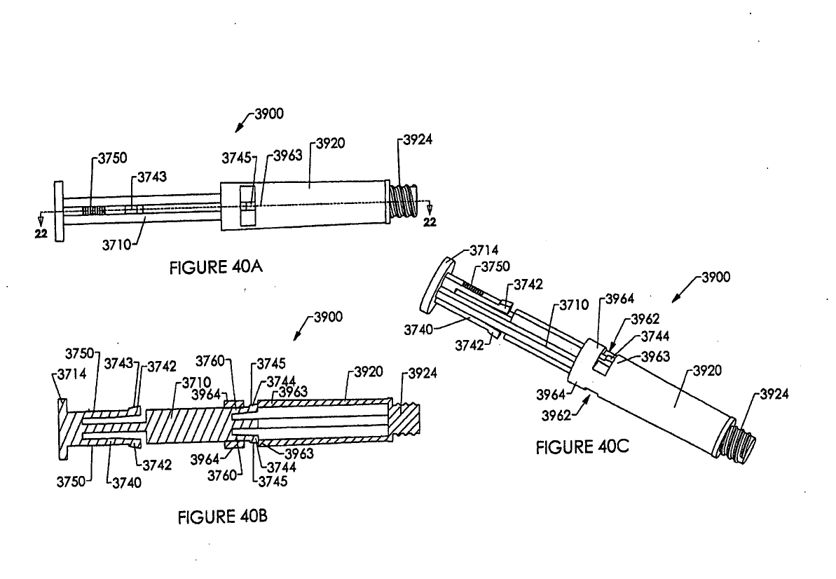

Referring now to FIGS. 39A to 39D and 40A to 40C, illustrated therein is an extensible plunger 3900 according to another embodiment. Plunger 3900 is substantially similar to plunger 3700, but it has a modified sleeve 3920. Sleeve 3920 is similar to sleeve 3720 in that it comprises a cruciform shaped cavity for receiving shaft 3710, a male threaded end 3924, cavities 3962, a distal edge 3963 and a proximal edge 3964. A notable difference is that a substantial part of the outer surface of sleeve 3920 is substantially frustoconical for interfering with grips 172, as opposed to the substantially cruciform cross-sectional outside profile of sleeve 3720. For brevity of description, only the differences between plungers 3700 and 3900 are described.

The frustoconical taper of sleeve 3920 extends from the proximal end of sleeve 3920 toward the distal end thereof in a similar manner to sleeve 320. In the illustrated embodiment, the taper stops partway down shaft 3910, with the remaining portion of shaft 3910 being substantially cylindrical. Alternatively, the frustoconical taper may extend the full longitudinal length of shaft 3910. The degree of the taper of the outer surface is preferably, but not exclusively, about 0.5 degrees to 2 degrees, and more preferably about 1 degree. As shown, this taper is provided on the external surface of sleeve 3920, not internally.

The purpose of the taper is to create an increasingly tight interference fit of the outer surface of sleeve 3920 with the inwardly facing surface of the grips 172 (or grips 1072 or 3372) around base opening 173 of activation cap 160 (or 1060 or 3360) as plunger 3900 is driven distally in its first stroke. This increasing interference progressively resists distal movement of actuating piston 150 within cartridge 130 as plunger 3900 progresses in its first stroke. This movement resistance mitigates against an overly vigorous actuation of plunger 3900 during mixing of constituents A and B. Such a vigorous actuation may result in an over-actuation of central piston 140, which is undesirable for reasons previously described.

Now referring to FIGS. 41A to 41D and 42A to 42C, illustrated therein is an extensible plunger 4100 according to another embodiment. Plunger 4100 comprises a shaft 4110 and a sleeve 4120 similar to shaft 3710 and sleeve 3720, respectively. However, shaft 4110 has only one finger and the cooperating transverse cross-sections of shaft 4110 and sleeve 4120 are partly semi-circular in shape. For brevity of description, the differences between plungers 3700 and 4100 are emphasized in this description, rather than describing the various similarities.

Shaft 4110 comprises a flange 4114, a proximal finger 4140 having a proximal projecting portion 4142, and a distal finger 4160 having a distal projecting portion 4144. Both proximal projecting portion 4142 and distal projecting portion 4144 have tapered surfaces 4143 and 4145 respectively and lips 4152 and 4154 respectively. Proximal finger 4140 further includes a finger activation portion 4150. Sleeve 4120 comprises a male threaded end 4124, a cavity 4162, a distal edge 4163 and a proximal edge 4164.

The transverse cross-sections of shaft 4110 and sleeve 4120 have corresponding shapes differing from the cruciform shapes of shaft 3710 and sleeve 3720. In particular, shaft 4110 has a somewhat semi-circular cross-section, with a rectangular key 4170 on a flat longitudinal face of shaft 4110. Sleeve 4120 has a corresponding semi-circular shape with a rectangular keyway 4172 for receiving key 4170. Key 4170 and keyway 4172 cooperate to allow longitudinal movement of shaft 4110 relative to sleeve 4120 but prevent relative rotational movement therebetween. As shown, fingers 4140, 4160 are formed integrally with key 4170 in a similar manner to how fingers 3740, 3760 are formed integrally with shaft 3710.

In the illustrated embodiment of plunger 4100, the outer surface of shaft 4120 is substantially cylindrical. Alternatively, the outer surface may be tapered like sleeve 320, or sleeve 3920 or another shape, as long as the outer surface can be received within cartridge 130. Plungers 3700, 3900 and 4100 all have cooperating structures tending to prevent relative rotation of the sleeve and shaft, while allowing relative longitudinal movement between extended and retracted positions. This prevention of relative rotation assists in screwing the screw-threaded male end of the plunger into piston 150. Instead of such screw-threaded engagement, other, less preferred forms of engagement between the plunger and piston 150 may be employed.

In alternative embodiments of the extensible plungers described herein, the shaft and sleeve may be interchanged such that the sleeve is located on the more proximal of the two coaxial plunger members and the shaft is the more distal of the plunger members, having the male threaded end on its distal extremity. Cooperating locking and engaging structures of such embodiments will be apparent to those skilled in the art, based on the locking and engaging structures described herein.

Various embodiments of the invention have been described in relation to the drawings. However, some modifications may be made to the described embodiments, without departing from the spirit and scope of the invention. Further, various features, functions and elements described in relation to one or more of the embodiments may be used in conjunction with one or more of the other embodiments, except to the extent that such a combination would be unworkable.

Specifically, connector sleeve 1620, fluid connector 1640 and vial 1630 may be used with any of the described embodiments of the delivery device, rather than just with delivery device 1500. Those skilled in the art will appreciate that such alternative embodiments of the mixing device will require only trivial modifications to the structure of the distal end of the delivery device and/or substitution of an activation cap having a longer sleeve.

Further, alternative activation methods may be employed to accommodate such alternative mixing device embodiments. For example, a double-ended needle, such as needle 1080, may be used to replace spikes 1514 and 1642 of mixing device 1600. Further, any of the described plunger embodiments may be used with any of the delivery or mixing device embodiments.

Further, while preferred materials have been described for the various device/assembly components, some or all of the materials may be replaced with other suitable materials, provided that the appropriate form and function (as described herein) can be obtained with such replacement materials.

1. A device for delivery of a substance, comprising:
a socket member having a distal end and an open proximal end and a hollow body extending therebetween, the distal end being closed but for a fluid delivery passage therein;
a tubular container having a fluid communication end and an actuation end and being receivable in the socket member so that the fluid communication end is disposed toward the distal end of the socket member, the container having a first closure member disposed at the fluid communication end, a second closure member disposed at the actuation end, a piston within the container intermediate the first and second closure members and bypass means for enabling fluid to bypass the piston when the piston is in a bypass position;
an activation cap disposed around the actuation end of the container, the activation cap comprising a sleeve extending along a proximal portion of an outer surface of the container and being at least partly receivable within the proximal end of the socket member, the activation cap further comprising a base portion arranged to grip the actuation end of the container and to permit access to the second closure member through the actuation end; and
a plunger engageable with the second closure member for moving the second closure member within the container;
wherein, in a pre-activated state of the device, a first chamber is defined by the container, the second closure member and the piston, a second chamber is defined by the container, the piston and the first closure member and the first chamber is sealed from communication with the fluid delivery passage of the socket member and, in an activated state of the device, the fluid delivery passage is in fluid communication with the second chamber through the first closure member for delivery of the contents of the second chamber through the fluid delivery passage.

2. The device of claim 1, wherein the plunger is longitudinally extensible between a retracted position and an extended position.

3. The device of claim 2, wherein the plunger is adapted to be actuated in a first stroke in its retracted position and in a second stroke in its extended position.

4. The device of claim 3, wherein, in the pre-activated state of the device, the plunger is actuable in the first stroke and, in the activated state of the device, the plunger is actuable in the second stroke.

5. The device of claim 2, wherein the plunger comprises first and second coaxial members.

6. The device of claim 2, wherein the plunger is extensible between the retracted position and the extended position by movement of a shaft of the plunger relative to a sleeve of the plunger.

7. The device of claim 6, wherein the plunger has a screw-threaded distal portion for screw-threaded engagement with the second closure member.

8. The device of claim 6, wherein the shaft and sleeve have corresponding screw threads and extension of the plunger from the retracted position to the extended position is effected by a screw rotation of the shaft.

9. The device of claim 6, wherein the shaft is slidably received in the sleeve.

10. The device of claim 6, wherein the shaft and sleeve have cooperating portions tending to retain the plunger in the retracted position or the extended position.

11. The device of claim 6, wherein the shaft is substantially cylindrical and has a head portion having a larger diameter portion for cooperating with internal projections of the sleeve to prevent longitudinal detachment of the sleeve from the shaft.

12. The device of claim 1, wherein the plunger has a plurality of radial projections along a shaft thereof for interfering with the base portion of the activation cap during movement of the shaft and second closure member within the container to thereby retard such movement.

13. The device of claim 6, wherein the sleeve has a tapered outer surface.

14. The device of claim 13, wherein the taper angle of the tapered outer surface is between about 0.5 degrees and 2 degrees.

15. The device of claim 13, wherein in the first stroke of the plunger, the tapered outer surface of the sleeve progressively engages the base portion so as to provide progressively greater resistance to movement of the plunger in the first stroke.

16. The device of claim 1, wherein the second closure member has a distal projection and the piston has a proximal recess interlockingly engageable with the projection to enable the plunger to proximally withdraw the piston and the second closure member together in an aspiration action of the device.

17. The device of claim 1, wherein the device is movable from the pre-activated state to the activated state by moving the sleeve of the activation cap distally within the socket member so that the base portion of the activation cap is positioned adjacent the proximal end of the socket member.

18. The device of claim 1, wherein the socket member has penetration means disposed at the distal end thereof and directed generally proximally for penetrating the first closure member.

19. The device of claim 18, wherein the penetration means comprises a fluid passage for providing fluid communication of an external volume with an interior volume of the tubular container when the penetration means penetrates the first closure member.

20. The device of claim 18, wherein the penetration means comprises a spike integrally formed with the distal end of the socket member.

21. The device of claim 18, wherein the penetration means comprises a proximal end of a hollow needle received at least partly within the distal end of the socket member.

22. The device of claim 18, wherein the penetration means comprises a spike which, in a pre-activated state of the device, is slidably moveable in a distal direction.

23. The device of claim 1, wherein the distal end of the socket member comprises a cylindrical neck and lugs or threads disposed on an outer surface thereof for screw-threaded engagement with a female thread.

24. The device of claim 23, further comprising a connector sleeve having a female thread engageable with the lugs or threads and a fluid connection passage adapted to form a fluid connection between the fluid delivery passage and a vial received in a distal opening of the connector sleeve.

25. The device of claim 24, wherein, in the activated state of the device, a fluid connection is established between the vial via the fluid connection passage and the fluid delivery passage, whereby fluid is communicable between the second chamber and an interior volume of the vial.

26. The device of claim 24, wherein the vial defines a third chamber and the device is operable to mix the contents of the first, second and third chambers.

27. The device of claim 26, wherein the device is operable to mix the contents of the first and second chambers together and then mix the mixed contents with the contents of the third chamber.

28. The device of claim 1, wherein the tubular container has a neck portion disposed toward said fluid communication end and wherein the first closure member is disposed at least partly within the neck portion.

29. The device of claim 28, wherein the neck portion is substantially straight.

30. The device of claim 28, wherein the first closure member is a stopper received within the neck portion and having a recess therein extending distally from a proximal face of the stopper so as to define a thin central portion for penetration thereof by the penetration means.

31. The device of claim 26, wherein the first closure member is a piston received within the neck portion and having a recess therein extending distally from a proximal face of the piston so as to define a thin central portion for penetration thereof by the penetration means.

32. The device of claim 29, wherein the first closure member is engaged with a proximal protrusion of the distal end of the socket member.

33. The device of claim 32, wherein the first closure member is engaged in screw threaded connection with the proximal protrusion.

34. The device of claim 1, wherein the bypass means comprises at least one longitudinally extending recess formed in a wall of the tubular container.

35. The device of claim 1, wherein the bypass means comprises an interiorly directed deformation of a longitudinal portion of a wall of the tubular container.

36. The device of claim 35, wherein the deformation is created by forming an inwardly directed recess in an outer surface of the wall.

37. The device of claim 35, wherein in the bypass position, the piston is deformed by the interior deformation of the wall so as to partially separate the piston from the wall of the tubular container.

38. The device of claim 34, wherein the bypass means comprises a plurality of recesses formed in the wall of the tubular container.

39. The device of claim 38, wherein the recesses are evenly spaced around a circumference of the wall.

40. The device of claim 38, wherein the bypass means comprises two to six recesses.

41. The device of claim 34, wherein the wall extends outwardly from a nominal exterior surface of the wall at the position of each recess by about 0.3 mm or less.

42. The device of claim 34, wherein each recess has a curved cross-sectional profile.

43. A method of delivery of a substance, comprising the steps of:
providing a delivery device comprising a syringe socket, a cartridge containing separate first and second constituents and at least partly received in the syringe socket, an axially extensible plunger extensible between a retracted position and an extended position and fluid connection means for providing fluid communication between a volume internal of the syringe socket and an external volume thereof;
activating the delivery device by inserting a part of the fluid connection means into the cartridge and thereby establishing fluid communication between an internal volume of the cartridge and the external volume;
actuating the plunger in its retracted position, in a first stroke of the plunger, to cause the first constituent to mix with the second constituent and form the substance;
extending the plunger to the extended position; and
actuating the plunger in its extended position in a second stroke of the plunger to deliver the substance to the external volume via the fluid connection means.

44. The method of claim 43, wherein the delivery device further comprises an activation cap engaging a proximal end of the cartridge and at least partly received in the syringe socket and the step of activating includes moving the activation cap axially within the syringe socket to force insertion of the part of the fluid connection means into the cartridge.

45. The method of claim 44, further comprising retarding the plunger in its first stroke by interference of an outer surface of the plunger with a base portion of the activation cap.

46. The method of claim 43, wherein the step of extending comprises screw rotation of a first member of the plunger relative to a second member of the plunger.

47. The method of claim 43, wherein the step of extending comprises slidably moving a first member of the plunger relative to a second member of the plunger.

48. The method of claim 43, wherein the external volume is defined by a vial containing a third constituent and delivering the substance to the external volume mixes the substance with the third constituent, the method further comprising the step of aspirating the mixed substance and third constituent via the fluid connection means into the cartridge by activating the plunger in its extended position in a third stroke of opposite direction to the first and second strokes; selecting another external volume; and activating the plunger in its extended position in a fourth stroke of the same direction of the first and second strokes to deliver the mixed substance and third constituent to the other volume via the fluid connection means.

49. A device for delivery of a pharmaceutical substance, comprising:
a syringe socket having a fluid connection end;
a cartridge at least partly received in the syringe socket, the cartridge defining a first chamber containing a first constituent, a second chamber containing a second constituent and bypass means for fluidly connecting the first and second chambers to allow mixing of the first and second constituents;
a connection part comprising fluid communication means and first and second engaging portions, the first engaging portion being adapted to engage the syringe socket at the fluid connection end and the second engaging portion being adapted to engage a head portion of a container having a third chamber containing a third constituent, wherein, in an activated position of the device, the second chamber is in fluid communication with the fluid communication means via the fluid connection end, whereby, when the head portion of the container is engaged with the second engaging portion and the fluid communication means is in fluid communication with the third chamber, mixed first and second constituent is fluidly mixable with the third constituent to form the pharmaceutical substance; and
means for aspirating the third constituent into the cartridge for mixing and subsequent delivery of the pharmaceutical substance via the fluid connection end of the syringe socket.

50. A device for mixing constituents of a pharmaceutical substance, comprising:
first, second and third chambers defined by the device, the first, second and third chambers containing respective first, second and third constituents and being oriented in sequence along a longitudinal axis of the device;
wherein the device is arranged such that, in an activated position, the first, second and third constituents may be mixed by actuation of a plunger upon a piston associated with the first chamber.

51. The device of claim 50, wherein in the activated position, initial actuation of the plunger causes the first constituent to flow into the second chamber, thereby mixing the first and second constituents, and further actuation of the plunger causes mixed first and second constituent to flow into the third chamber and thereby mix the first and second constituent with the third constituent.

52. The device of claim 51, wherein still further actuation of the plunger causes mixed first, second and third constituent to flow into the second chamber.

53. The device of claim 50, wherein the first, second and third chambers are at least partly received within an elongate tubular housing.

54. The device of claim 53, wherein the housing comprises first and second detachable parts, the first detachable part at least partly receiving the first and second chambers and the second detachable part at least partly receiving the third chamber.

55. A method of mixing first and second constituents of a substance, comprising the steps of:
providing a delivery device comprising a socket assembly, a cartridge separately containing the first and second constituents and at least partly received in the socket assembly, a plunger engageable with a closure member of the cartridge and fluid connection means for providing fluid communication between a volume internal of the socket assembly and an external volume thereof the socket assembly having a base portion engaging a proximal end of the cartridge and defining a base opening;
activating the delivery device by inserting a part of the fluid connection means into the cartridge and thereby establishing fluid communication between an internal volume of the cartridge and the external volume;
actuating the plunger to move the closure member within the cartridge and to thereby cause the first constituent to mix with the second constituent and form the substance, such mixing being performed in a first stroke of the plunger; and
interfering the base portion of the socket assembly with an outer surface of the plunger during actuation of the plunger to retard movement of the plunger and the closure member during the first stroke.

56. The method of claim 55, further comprising the step of:
actuating the plunger in a second stroke to deliver the substance to the external volume.

57. The method of claim 56, further comprising the step of:
allowing uninterfered movement of the plunger relative to the base portion of the socket assembly in the second stroke.

58. The method of claim 55, wherein the external volume is a vial.

59. The method of claim 55, wherein the external volume is a patient body.

60. The method of claim 55, wherein the plunger is extensible and the method further comprises extending the plunger from a retracted position to an extended position and actuating the plunger in a second stroke to deliver the substance to the external volume.

61. The method of claim 60, further comprising the step of:
allowing uninterfered movement of the plunger relative to the base portion of the socket assembly in the second stroke.

62. The method of claim 55, wherein the socket assembly comprises a syringe socket and an activation cap at least partly received in the syringe socket, the activation cap having the base portion.
